(19) United States
(12) Patent Application Publication
(10) Pub. No.: US 2025/0355121 A1
(43) Pub. Date: Nov. 20, 2025

(54) RADIOGRAPHIC IMAGING APPARATUS AND RADIOGRAPHIC IMAGING SYSTEM

(71) Applicant: CANON KABUSHIKI KAISHA, Tokyo (JP)

(72) Inventors: HIROSHI SASAKI, Kanagawa (JP); SHINSUKE HAYASHIDA, Tokyo (JP); YOSHITAKA OTSUBO, Kanagawa (JP); YURI YOSHIMURA, Tokyo (JP); RIKUTO MASUDA, Kanagawa (JP); ERIKO SATO, Tokyo (JP)

(21) Appl. No.: 19/176,848

(22) Filed: Apr. 11, 2025

Related U.S. Application Data
(63) Continuation of application No. PCT/JP2023/037127, filed on Oct. 13, 2023.

(30) Foreign Application Priority Data
Oct. 14, 2022 (JP) 2022-165498
Oct. 27, 2022 (JP) 2022-172565
Apr. 10, 2023 (JP) 2023-063673
Apr. 25, 2023 (JP) 2023-071786
Jul. 24, 2023 (JP) 2023-119938
Oct. 3, 2023 (JP) 2023-171786

(51) Int. Cl. G01T 1/20; G01T 1/166

(52) U.S. Cl. CPC G01T 1/20181; G01T 1/1663; G01T 1/20188

(57) ABSTRACT

A radiation detection panel, a control substrate, a processing substrate, and a housing, wherein the housing includes a thin section, having a first thickness in an incident direction of the radiation, where the effective imaging area is disposed, and a thick section, having a second thickness greater than the first thickness in the incident direction of the radiation, where the control substrate and the processing substrate are disposed, and wherein the control substrate and the processing substrate disposed in the thick section overlap at least in part as seen in the incident direction of the radiation.

### CROSS-REFERENCE TO RELATED APPLICATIONS

This application is a Continuation of International Patent Application No. PCT/JP2023/037127, filed Oct. 13, 2023, which claims the benefit of Japanese Patent Applications No. 2022-165498, filed Oct. 14, 2022, No. 2022-172565, filed Oct. 27, 2022, No. 2023-063673, filed Apr. 10, 2023, No. 2023-071786, filed Apr. 25, 2023, No. 2023-119938, filed Jul. 24, 2023, No. 2023-171786, filed Oct. 3, 2023, which are hereby incorporated by reference herein in their entirety.

### BACKGROUND OF THE INVENTION

### Field of the Invention

The present invention relates to a radiographic imaging apparatus and a radiographic imaging system.

### Background Art

Radiographic imaging apparatuses that detect an intensity distribution of radiation transmitted through a subject to obtain a radiographic image are widely and commonly used in the field of medical diagnosis. To enable quick imaging of a wide range of body parts, such radiographic imaging apparatuses are demanded to be thin and easy to handle. In view of such a demand, Patent Literature (PTL) 1 discusses a radiographic imaging apparatus including a housing with a thin section where a radiation detection panel is disposed and a thick section where a plurality of components such as a control substrate and a power supply is disposed. PTL 2 discusses a radiographic imaging apparatus including a thin first housing where a radiation detection panel is disposed and a second housing that is separate from the first housing and configured to be movable on the first substrate and where a plurality of components such as a control substrate and a power supply is disposed.

### CITATION LIST

### Patent Literature

PTL 1: International Publication Number WO2020/105706
PTL 2: Japanese Patent No. 5638372

If a plurality of components is arranged in a planar direction as in the thick section of the housing discussed in PTL1 or the second housing discussed in PTL2, there is an issue that the thick section of the radiographic imaging apparatus becomes large in the planar direction. In other words, conventional radiographic imaging apparatuses have had an issue of being insufficient for performing appropriate operation with an appropriate shape in consideration of the user's workability.

### SUMMARY OF THE INVENTION

The present invention has been achieved in the foregoing issue, and is directed to providing a radiographic imaging apparatus that enables appropriate operation with an appropriate shape in consideration of the user's workability.

According to an aspect of the present disclosure, a radiographic imaging apparatus includes a radiation detection panel configured to include an effective imaging area where incident radiation is detected, a control substrate configured to control driving of the radiation detection panel, a processing substrate configured to process a signal output from the radiation detection panel, and a housing configured to accommodate the radiation detection panel, the control substrate, and the processing substrate, wherein the housing includes a first thickness section, which has a first thickness in an incident direction of the radiation, and where the effective imaging area is disposed, and a second thickness section, which has a second thickness greater than the first thickness in the incident direction of the radiation, and where the control substrate and the processing substrate are disposed, and wherein the control substrate and the processing substrate are disposed to overlap each other at least in part when the second thickness section is viewed along the incident direction of the radiation.

According to another aspect of the present disclosure, a radiographic imaging apparatus includes a radiation detection panel configured to include an effective imaging area where incident radiation is detected, a control substrate configured to control driving of the radiation detection panel, a housing configured to accommodate the radiation detection panel and the control substrate, and a grip portion configured to be gripped to hold the housing, wherein the housing includes a first thickness section, which has a first thickness in an incident direction of the radiation, and where the effective imaging area is disposed, and a second thickness section, which has a second thickness greater than the first thickness in the incident direction of the radiation, and where the control substrate and the grip portion are disposed, and wherein the control substrate and the grip portion are disposed to overlap each other at least in part when the second thickness section is viewed along the incident direction of the radiation, and the control substrate is disposed at a position closer to a side where the radiation is incident than the grip portion.

According to yet another aspect of the present disclosure, a radiographic imaging apparatus includes a radiation detection panel configured to include an effective imaging area where incident radiation is detected, a control substrate configured to control driving of the radiation detection panel, a flexible circuit board configured to connect the radiation detection panel and the control substrate, and a housing configured to accommodate the radiation detection panel, the control substrate, and the flexible circuit board, wherein the housing includes a first thickness section, which has a first thickness in an incident direction of the radiation, and where the effective imaging area is disposed, a second thickness section, which has a second thickness greater than the first thickness in the incident direction of the radiation, and where the control substrate is disposed, and a gradient section, which connects the first thickness section and the second thickness section with a gradient, and where at least a part of the flexible circuit board is disposed, and wherein the flexible circuit board connects the radiation detection panel and the control substrate which are disposed at different positions in the incident direction of the radiation, with a gradient.

According to yet another aspect of the present disclosure, a radiographic imaging apparatus includes a radiation detection panel configured to include an effective imaging area where radiation transmitted through a subject is detected, a predetermined circuit configured to detect a signal output from the radiation detection panel, and a housing configured to accommodate the radiation detection panel and the predetermined circuit. wherein the housing includes a first thickness section, which has a first thickness in an incident direction of the radiation, and where at least the effective imaging area is disposed, and a second thickness section, which has a second thickness greater than the first thickness in the incident direction of the radiation, and where at least the predetermined circuit is disposed, and wherein in the second thickness section, a current reduction mechanism for reducing a loop current in a region where a closed circuit may occur is disposed.

According to yet another aspect of the present disclosure, a radiographic imaging apparatus includes a radiation detection panel configured to include an effective imaging area where incident radiation is detected, a housing configured to accommodate the radiation detection panel, and a display unit configured to function as a user interface, wherein the housing includes a first thickness section, which has a first thickness in an incident direction of the radiation, and where the effective imaging area is disposed, and a second thickness section, which has a second thickness greater than the first thickness in the incident direction of the radiation, and where the display unit is disposed in an area which is excluded from a center in a longitudinal direction and is on one end side in the longitudinal direction.

According to yet another aspect of the present disclosure, a radiographic imaging apparatus includes a radiation detection panel configured to include an effective imaging area where radiation transmitted through a subject is detected, a sensor unit configured to include one or more types of sensors for detecting the subject, and a housing configured to accommodate the radiation detection panel, wherein the housing includes a first thickness section, which has a first thickness in an incident direction of the radiation, and where the effective imaging area is disposed, and a second thickness section, which has a second thickness greater than the first thickness in the incident direction of the radiation, and where the sensor unit is disposed.

According to yet another aspect of the present disclosure, a radiographic imaging apparatus includes a radiation detection panel configured to include an effective imaging area where radiation transmitted through a subject is detected, and a housing configured to accommodate the radiation detection panel, wherein in the housing, an index indicating a range of the effective imaging area is disposed on a first surface corresponding to a surface on one side of the radiation detection panel and a second surface corresponding to a surface on the other side of the radiation detection panel.

Further features of the present invention will become apparent from the following description of exemplary embodiments with reference to the attached drawings.

### BRIEF DESCRIPTION OF THE DRAWINGS

FIG. 1 is a diagram illustrating an example of a schematic configuration of a radiographic imaging system according to a first exemplary embodiment.

FIG. 2 is a diagram illustrating an example of an internal configuration in cross section A-A of a radiographic imaging apparatus according to the first exemplary embodiment illustrated in FIG. 1.

FIG. 3 is a view of components inside a housing of the radiographic imaging apparatus according to the first exemplary embodiment, seen from the rear.

FIG. 4 is a diagram illustrating an example of a schematic configuration of a radiographic imaging system according to a second exemplary embodiment.

FIG. 5 is a view of a radiographic imaging apparatus according to the second exemplary embodiment, seen from the rear.

FIG. 6 is a diagram illustrating an example of an internal configuration in cross section B-B of the radiographic imaging apparatus according to the second exemplary embodiment illustrated in FIG. 5.

FIG. 7 is a diagram illustrating an example of a schematic configuration of a radiographic imaging system according to a third exemplary embodiment.

FIG. 8 is a diagram illustrating an example of an internal configuration in cross section C-C of a radiographic imaging apparatus according to the third exemplary embodiment illustrated in FIG. 7.

FIG. 9 is a schematic perspective view illustrating the appearance of a typical radiographic imaging apparatus.

FIG. 10 is a schematic sectional view taken along dot-dashed line D-D′ in FIG. 9.

FIG. 11 is a schematic sectional view illustrating a typical configuration of a radiographic imaging apparatus.

FIG. 12 is a schematic plan view illustrating structural elements of the typical radiographic imaging apparatus as seen from the rear in a radiation incident direction.

FIG. 13A is a schematic enlarged plan view of a region R surrounded by a broken line in FIG. 12.

FIG. 13B is a schematic enlarged plan view of the region R surrounded by a broken line in FIG. 12.

FIG. 14A is a schematic diagram illustrating a radiographic imaging apparatus including a current reduction mechanism according to a first aspect of a fourth exemplary embodiment.

FIG. 14B is a schematic diagram illustrating the radiographic imaging apparatus including the current reduction mechanism according to the first aspect of the fourth exemplary embodiment.

FIG. 15A is a schematic diagram illustrating a radiographic imaging apparatus including a current reduction mechanism according to another example of the first aspect of the fourth exemplary embodiment.

FIG. 15B is a schematic diagram illustrating a radiographic imaging apparatus including a current reduction mechanism according to another example of the first aspect of the fourth exemplary embodiment.

FIG. 16 is a schematic enlarged plan view of the region R where a current reduction mechanism according to a second aspect is disposed in the radiographic imaging apparatus according to the fourth exemplary embodiment.

FIG. 17A is a schematic view illustrating a current reduction mechanism according to a third exemplary embodiment along with a typical radiographic imaging apparatus, illustrating a state where a closed circuit is formed in the radiographic imaging apparatus according to the fourth exemplary embodiment.

FIG. 17B is a schematic view illustrating the current reduction mechanism according to the third exemplary embodiment along with the typical radiographic imaging apparatus, illustrating a state where a closed circuit is formed in the radiographic imaging apparatus according to the fourth exemplary embodiment.

FIG. 18A is a schematic diagram illustrating the current reduction mechanism according to the third aspect along with a typical radiographic imaging apparatus, illustrating a state where a loop current occurs in the radiographic imaging apparatus according to the fourth exemplary embodiment.

FIG. 18B is a schematic diagram illustrating the current reduction mechanism according to the third aspect along with the typical radiographic imaging apparatus, illustrating a state where a loop current occurs in the radiographic imaging apparatus according to the fourth exemplary embodiment.

FIG. 19 is a schematic plan view of a typical configuration of a radiographic imaging apparatus according to a fifth exemplary embodiment, as seen from the rear in a radiation incident direction.

FIG. 20 is a schematic plan view illustrating a radiographic imaging apparatus including a current reduction mechanism according to a first aspect of the fifth exemplary embodiment.

FIG. 21 is a schematic plan view illustrating a radiographic imaging apparatus including a current reduction mechanism according to a second aspect of the fifth exemplary embodiment.

FIG. 22 is a schematic plan view of a typical configuration of a radiographic imaging apparatus according to a sixth exemplary embodiment, as seen from the rear in a radiation incident direction.

FIG. 23 is a schematic plan view illustrating a radiographic imaging apparatus including a current reduction mechanism according to a first aspect of the sixth exemplary embodiment.

FIG. 24 is a schematic plan view illustrating a radiographic imaging apparatus including a current reduction mechanism according to a second aspect of the sixth exemplary embodiment.

FIG. 25 is a schematic diagram illustrating a seventh exemplary embodiment, illustrating a radiographic imaging system including any of the radiographic imaging apparatuses according to the first to third aspects of the fourth to sixth exemplary embodiments.

FIG. 26 is a diagram illustrating an example of a schematic configuration of a radiographic imaging system according to an eighth exemplary embodiment.

FIG. 27 is a diagram illustrating an example of the appearance of a radiographic imaging apparatus according to the eighth exemplary embodiment.

FIG. 28 is a diagram illustrating an example of a functional configuration of the radiographic imaging apparatus according to the eighth exemplary embodiment.

FIG. 29A is a diagram for explaining a selection example of ROIs to be used for AEC using a display unit on the radiographic imaging apparatus according to the eighth exemplary embodiment.

FIG. 29B is a diagram for explaining a selection example of ROIs to be used for AEC using the display unit on the radiographic imaging apparatus according to the eighth exemplary embodiment.

FIG. 30 is a flowchart illustrating an example of a processing procedure for a radiographic imaging method of a radiographic imaging system according to a ninth exemplary embodiment.

FIG. 31A is a diagram illustrating a display example of a display unit on a radiographic imaging apparatus according to the ninth exemplary embodiment.

FIG. 31B is a diagram illustrating a display example of the display unit on the radiographic imaging apparatus according to the ninth exemplary embodiment.

FIG. 31C is a diagram illustrating a display example of the display unit on the radiographic imaging apparatus according to the ninth exemplary embodiment.

FIG. 31D is a diagram illustrating a display example of the display unit on the radiographic imaging apparatus according to the ninth exemplary embodiment.

FIG. 31E is a diagram illustrating a display example of the display unit on the radiographic imaging apparatus according to the ninth exemplary embodiment.

FIG. 31F is a diagram illustrating a display example of the display unit on the radiographic imaging apparatus according to the ninth exemplary embodiment.

FIG. 32A is a diagram illustrating an example of the appearance of a radiographic imaging apparatus according to a tenth exemplary embodiment.

FIG. 32B is a diagram illustrating an example of the appearance of the radiographic imaging apparatus according to the tenth exemplary embodiment.

FIG. 33 is a diagram illustrating an example of the appearance of a radiographic imaging apparatus according to an eleventh exemplary embodiment.

FIG. 34 is a diagram illustrating an example of the appearance of a radiographic imaging apparatus according to a twelfth exemplary embodiment.

FIG. 35 is a diagram illustrating an example of a schematic configuration of a radiographic imaging system according to a thirteenth exemplary embodiment.

FIG. 36A is a diagram illustrating an example of an internal configuration in cross section F-F of a radiographic imaging apparatus illustrated in FIG. 35.

FIG. 36B is a diagram illustrating the example of the internal configuration in cross section F-F of the radiographic imaging apparatus illustrated in FIG. 35.

FIG. 37 is a flowchart illustrating an example of a processing procedure for a control method of the radiographic imaging apparatus according to the thirteenth exemplary embodiment.

FIG. 38 is a diagram illustrating an example of an internal configuration of the radiographic imaging apparatus according to the thirteenth exemplary embodiment.

FIG. 39 is a diagram illustrating modification 1 of a schematic configuration of the radiographic imaging apparatus according to the thirteenth exemplary embodiment.

FIG. 40 is a diagram illustrating modification 2 of a schematic configuration of the radiographic imaging apparatus according to the thirteenth exemplary embodiment.

FIG. 41A is a diagram illustrating an example of the internal configuration of the radiographic imaging apparatus according to the thirteenth exemplary embodiment.

FIG. 41B is a diagram illustrating an example of the internal configuration of the radiographic imaging apparatus according to the thirteenth exemplary embodiment.

FIG. 42A is a diagram illustrating an example of an internal configuration of a radiographic imaging apparatus according to a fourteenth exemplary embodiment.

FIG. 42B is a diagram illustrating an example of the internal configuration of the radiographic imaging apparatus according to the fourteenth exemplary embodiment.

FIG. 43A is a diagram illustrating an example of an internal configuration of a radiographic imaging apparatus according to a fifteenth exemplary embodiment.

FIG. 43B is a diagram illustrating an example of the internal configuration of the radiographic imaging apparatus according to the fifteenth exemplary embodiment.

FIG. 44 is a diagram illustrating an example of an internal configuration of a radiographic imaging apparatus according to a sixteenth exemplary embodiment.

FIG. 45 is a diagram illustrating an example of an internal configuration of a radiographic imaging apparatus according to a seventeenth exemplary embodiment.

FIG. 46 is a chart illustrating examples of detection capabilities of sensors applied in the thirteenth to seventeenth exemplary embodiments.

FIG. 47 is a flowchart illustrating an example of a processing procedure for a control method of a radiographic imaging apparatus according to an eighteenth exemplary embodiment.

FIG. 48 is a diagram illustrating an example of a schematic configuration of a radiographic imaging apparatus according to a nineteenth exemplary embodiment.

FIG. 49A is a diagram illustrating a first example of identifying the position of a subject on the radiographic imaging apparatus according to the nineteenth exemplary embodiment.

FIG. 49B is a diagram illustrating the first example of identifying the position of a subject on the radiographic imaging apparatus according to the nineteenth exemplary embodiment.

FIG. 50A is a diagram illustrating a second example of identifying the position of a subject on the radiographic imaging apparatus according to the nineteenth exemplary embodiment.

FIG. 50B is a diagram illustrating the second example of identifying the position of a subject on the radiographic imaging apparatus according to the nineteenth exemplary embodiment.

FIG. 51 is a flowchart illustrating an example of a processing procedure for a control method of the radiographic imaging apparatus according to the nineteenth exemplary embodiment.

FIG. 52 is a diagram illustrating an example of a part of the schematic configuration of a radiographic imaging apparatus according to a twentieth exemplary embodiment.

FIG. 53 is a diagram illustrating a first example of the schematic configuration of the radiographic imaging apparatus according to the twentieth exemplary embodiment.

FIG. 54 is a diagram illustrating a second example of the schematic configuration of the radiographic imaging apparatus according to the twentieth exemplary embodiment.

FIG. 55 is a diagram illustrating an example of a schematic configuration of a radiographic imaging apparatus according to a twenty-first exemplary embodiment.

FIG. 56 is a flowchart illustrating an example of a processing procedure from a start to an end of radiographic imaging of a subject using the radiographic imaging apparatus illustrated in FIG. 55.

FIG. 57A-1 is a diagram for describing the principle behind differences in image quality characteristics when radiographic images are captured with radiation incident on the front surface and the rear surface of a housing of an FPD imaging unit illustrated in FIG. 55.

FIG. 57A-2 is a diagram for describing the principle behind differences in image quality characteristics when radiographic images are captured with radiation incident on the front surface and the rear surface of a housing of an FPD imaging unit illustrated in FIG. 55.

FIG. 57B-1 is a diagram for describing the principle behind differences in image quality characteristics when radiographic images are captured with radiation incident on the front surface and the rear surface of a housing of an FPD imaging unit illustrated in FIG. 55.

FIG. 57B-2 is a diagram for describing the principle behind differences in image quality characteristics when radiographic images are captured with radiation incident on the front surface and the rear surface of a housing of an FPD imaging unit illustrated in FIG. 55.

FIG. 58A is a diagram illustrating an example of an operation screen displayed on an operation panel illustrated in FIG. 55.

FIG. 58B is a diagram illustrating an example of an operation screen displayed on an operation panel illustrated in FIG. 55.

FIG. 58C is a diagram illustrating an example of an operation screen displayed on an operation panel illustrated in FIG. 55.

FIG. 58D is a diagram illustrating an example of an operation screen displayed on an operation panel illustrated in FIG. 55.

FIG. 59A is a diagram illustrating an example of the appearance of the FPD imaging unit illustrated in FIG. 55.

FIG. 59B is a diagram illustrating an example of the appearance of the FPD imaging unit illustrated in FIG. 55.

FIG. 60A is a diagram illustrating a cross-sectional example of the FPD imaging unit illustrated in FIG. 55.

FIG. 60B is a diagram illustrating a cross-sectional example of the FPD imaging unit illustrated in FIG. 55.

FIG. 61 is a diagram illustrating a configuration example of the housing of the FPD imaging unit illustrated in FIG. 55.

FIG. 62 is a diagram illustrating a configuration example of the housing of the FPD imaging unit illustrated in FIG. 55.

FIG. 63A is a flowchart illustrating an example of a processing procedure for a control method of the radiographic imaging apparatus according to the twenty-first exemplary embodiment.

FIG. 63B is a flowchart illustrating an example of a processing procedure for a control method of the radiographic imaging apparatus according to a comparative example.

FIG. 64 is a diagram illustrating an example of image processing by image processing means according to the twenty-first exemplary embodiment and the comparative example.

FIG. 65A is a diagram illustrating an example of the appearance and internal configuration of the FPD imaging unit illustrated in FIG. 55.

FIG. 65B is a diagram illustrating an example of the appearance and internal configuration of the FPD imaging unit illustrated in FIG. 55.

FIG. 66A is a diagram illustrating the twenty-first exemplary embodiment and intended to describe a radiation incident direction determination method using light-shielded pixels illustrated in FIGS. 65A and 65B.

FIG. 66A-1 is a diagram illustrating the twenty-first exemplary embodiment and intended to describe a radiation incident direction determination method using light-shielded pixels illustrated in FIGS. 65A and 65B.

FIG. 66A-2 is a diagram illustrating the twenty-first exemplary embodiment and intended to describe a radiation incident direction determination method using light-shielded pixels illustrated in FIGS. 65A and 65B.

FIG. 66B is a diagram illustrating the twenty-first exemplary embodiment and intended to describe a radiation incident direction determination method using light-shielded pixels illustrated in FIGS. 65A and 65B.

FIG. 66B-1 is a diagram illustrating the twenty-first exemplary embodiment and intended to describe a radiation incident direction determination method using light-shielded pixels illustrated in FIGS. 65A and 65B.

FIG. 66B-2 is a diagram illustrating the twenty-first exemplary embodiment and intended to describe a radiation incident direction determination method using light-shielded pixels illustrated in FIGS. 65A and 65B.

FIG. 67 is a diagram illustrating an example of a processing procedure for the radiation incident direction determination processing by the radiographic imaging apparatus illustrated in FIG. 55.

FIG. 68 is a diagram illustrating specific examples of imaging systems to which the radiographic imaging apparatus according to the twenty-first exemplary embodiment is applicable.

### DESCRIPTION OF THE EMBODIMENTS

Hereinafter, modes (exemplary embodiments) for carrying out the present invention will be described with reference to the drawings. However, the details of the dimensions and structures shown in the exemplary embodiments are not limited to those described in the present specification and shown in the drawings. In this specification, the radiation includes not only X-rays but also α-rays, β-rays, γ-rays, particle beams, cosmic rays, and the like.

### First Exemplary Embodiment

A first exemplary embodiment will initially be described.

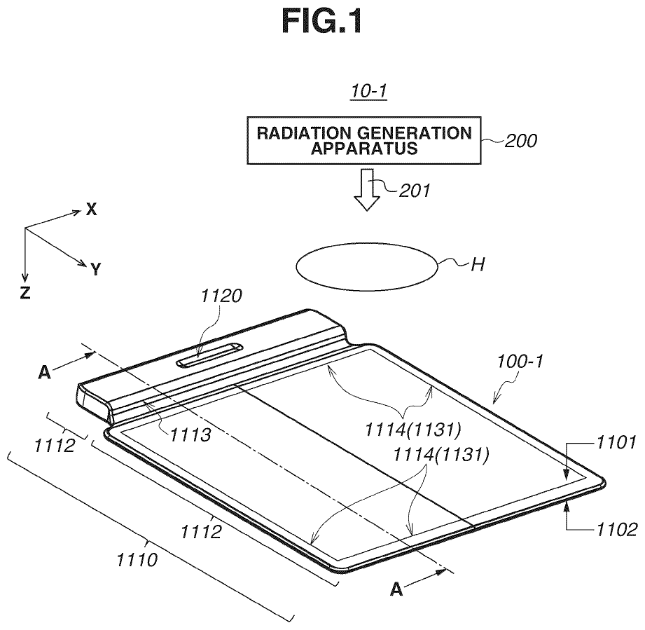

FIG. 1 is a diagram illustrating an example of a schematic configuration of a radiographic imaging system 10-1 according to the first exemplary embodiment. As illustrated in FIG. 1, the radiographic imaging system 10-1 includes a radiographic imaging apparatus 100-1 and a radiation generation apparatus 200.

The radiation generation apparatus 200 is an apparatus that emits radiation 201 toward a subject H and the radiographic imaging apparatus 100-1.

The radiographic imaging apparatus 100-1 is an apparatus that detects the incident radiation 201 (including the radiation 201 transmitted through the subject H) to obtain a radiographic image of the subject H. The radiographic image obtained by this radiographic imaging apparatus 100-1 is transmitted to an external apparatus, displayed on a monitor by the external apparatus, and used for diagnosis or the like, for example. FIG. 1 illustrates a radiation incident surface 1101 of the radiographic imaging apparatus 100-1 where the radiation is incident, and a rear surface 1102 opposite to the radiation incident surface 1101. FIG. 1 also illustrates an XYZ coordinate system with the incident direction of the radiation 201 (vertical direction) as a Z direction, and two mutually orthogonal directions orthogonal to the Z direction as an X direction and a Y direction.

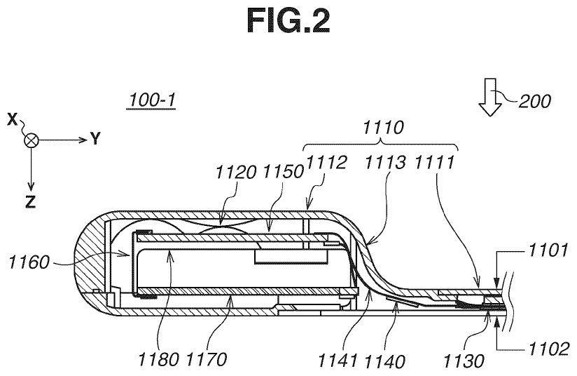

FIG. 1 illustrates a housing 1110 of the radiographic imaging apparatus 100-1 as the appearance of the radiographic imaging apparatus 100-1. An indicator 1114 indicating the range of an effective imaging area 1131 where a radiation detection panel (radiation detection panel 1130 of FIG. 2 to be described below) accommodated in the housing 1110 detects the radiation 201 transmitted through the subject H is displayed on the housing 1110.

As illustrated in FIG. 1, the housing 1110 includes a thin section 1111 corresponding to a first thickness section that is a section including the effective imaging area 1131 and has a first thickness in the Z direction that is the incident direction of the radiation 201. As illustrated in FIG. 1, the housing 1110 also includes a thick section 1112 corresponding to a second thickness section that is a section not including the effective imaging area 1131 and has a second thickness greater than the thickness (first thickness) of the thin section 1111 in the Z direction that is the incident direction of the radiation 201. More specifically, in the example illustrated in FIG. 1, the thick section (second thickness section) 1112 is thicker than the thin section (first thickness section) 1111 toward the side where the radiation 201 is incident. As illustrated in FIG. 1, the housing 1110 further includes a gradient section 1113 that connects the thin section (first thickness section) 1111 and the thick section (second thickness section) 1112 with a gradient. The housing 1110 is a single- or multi-part integral housing including the thin section (first thickness section) 1111, the thick section (second thickness section) 1112, and the gradient section 1113 described above. The thick section (second thickness section) 1112 of the housing 1110 is provided with a grip portion 1120 for the user to grip the housing 1110.

The housing 1110 illustrated in FIG. 1 will now be described in more detail.

To achieve portability and strength in a compatible manner, the housing 1110 is desirably formed of materials such as magnesium alloys, aluminum alloys, and fiber-reinforced plastic, for example. However, in the present exemplary embodiment, the housing 1110 may be formed of materials other than those mentioned here. In particular, the radiation incident surface 1101 of the thin section 1111 where the effective imaging area 1131 is disposed is desirably formed of a carbon fiber-reinforced plastic or the like with high transmittance for the radiation 201 and excellent lightweight properties, but other materials may also be used. Here, when imaging the subject H such as a patient using the radiation 201, the radiographic imaging apparatus 100-1 may be placed immediately behind the imaging site of the subject H. In doing so, due to a step created by the thickness of the housing 1110 of the radiographic imaging apparatus 100-1, the subject H and the end portion of the housing 1110 come into contact to cause a reaction force, and the patient or the like who is the subject H may feel discomfort. Typical radiographic imaging apparatuses are often provided in sizes compliant with ISO (International Organization for Standardization) 4090:2010, and often configured with a thickness of approximately 15 mm to 16 mm. By contrast, in the radiographic imaging apparatus 100-1 according to the present exemplary embodiment, the thin section 1111 of the housing 1110 has a thickness (first thickness) of 8.0 mm, for example. With the radiographic imaging apparatus 100-1 according to the present exemplary embodiment, the step created by the thickness of the housing 1110 during radiographic imaging is therefore smaller, and the reaction force occurring between the subject H and the end portion of the housing 1110 is reduced. To obtain such effects, the thickness of the thin section 1111 of the housing 1110 does not need to be limited to 8.0 mm, and may be even smaller, for example. The applicant has confirmed that the foregoing effects is obtainable if the thickness of the housing 1110 is less than 10.0 mm. In the present exemplary embodiment, the foregoing thickness of the thin section 1111 of the housing 1110 is set to 8.0 mm as an appropriate thickness in view of the configuration and mechanical strength of the radiation detection panel disposed in the thin section 1111.

FIG. 2 is a diagram illustrating an example of an internal configuration in cross section A-A of the radiographic imaging apparatus 100-1 according to the first exemplary embodiment illustrated in FIG. 1. In this FIG. 2, components similar to those illustrated in FIG. 1 are denoted by the same reference numerals, and a detailed description thereof will be omitted. FIG. 2 also illustrates an XYZ coordinate system corresponding to the XYZ coordinate system illustrated in FIG. 1. Specifically, cross section A-A illustrated in FIG. 1 is a cross section along the Y direction.

As illustrated in FIG. 2, the housing 1110 of the radiographic imaging apparatus 100-1 accommodates the radiation detection panel 1130, flexible circuit boards 1140, a control substrate 1150, wiring 1160, a processing substrate 1170, and a shielding member 1180. As described above, the thick section 1112 of the housing 1110 is provided with the grip portion 1120 for the user to grip the housing 1110.

In the example illustrated in FIG. 2, the grip portion 1120 is formed in a recessed shape in the side of the thick section 1112 of the housing 1110 where the radiation 201 is incident.

The radiation detection panel 1130 has the effective imaging area 1131 illustrated in FIG. 1, where the radiation 201 emitted from the radiation generation apparatus 200 and incident thereon (including the radiation 201 transmitted through the subject H) is detected. For example, the radiation detection panel 1130 may be configured using a so-called indirect conversion system, including a sensor substrate on which a large number of photoelectric conversion elements (sensors) are arranged, and a phosphor layer (scintillator layer), a phosphor protective film, and the like that are disposed above the sensor substrate. Here, the sensor substrate may be formed of materials such as glass and flexible plastic. However, in the present exemplary embodiment, the materials are not limited thereto. The phosphor protective film is formed of a material with low moisture permeability, and used to protect the phosphor layer. In the radiation detection panel 1130 of this indirect conversion system, the incident radiation 201 is converted into light in the phosphor layer, and the light obtained from the phosphor layer is converted into electrical signals by the respective photoelectric conversion elements, whereby image signals related to a radiographic image are generated. The radiation detection panel 1130 includes some or all of the photoelectric conversion elements (sensors) in its effective imaging area 1131. The effective imaging area 1131 is an area that is capable of radiographic imaging of the subject H and where radiographic images are actually generated. As illustrated in FIG. 1, the effective imaging area 1131 of the radiation detection panel 1130 is disposed within the thin section 1111. In the example illustrated in FIG. 1, the effective imaging area 1131 has a substantially rectangular shape as seen in the incident direction of the radiation 201. However, the present exemplary embodiment is not limited to the configuration illustrated in this FIG. 1. The radiation detection panel 1130 is not limited to the configuration of the foregoing indirect conversion system, either. For example, the radiation detection panel 1130 may be configured using a so-called direct conversion system, including a conversion element unit where conversion elements formed of a-Se or the like and switch elements such as TFTs are two-dimensionally arranged. In the radiation detection panel 1130 of this direct conversion system, the incident radiation 201 is converted into electrical signals by the respective conversion elements, whereby image signals related to a radiographic image are generated.

The flexible circuit boards 1140 are boards that connect the radiation detection panel 1130 and the control substrate 1150. As illustrated in FIG. 2, the radiation detection panel 1130 and the control substrate 1150 are disposed at different positions (heights) in the Z direction that is the incident direction of the radiation 201. The flexible circuit boards 1140 thus connect the radiation detection panel 1130 and the control substrate 1150 with a gradient 1141 with respect to the Y direction that is a horizontal direction. As illustrated in FIG. 2, the flexible circuit boards 1140 are disposed at least in part in the gradient section 1113 of the housing 1110. The flexible circuit boards 1140 include various substrates and elements inside, and thus need a predetermined area. This makes the radiographic imaging apparatus 100-1 large in the planar direction (plane including the Y direction) if the flexible circuit boards 1140 are disposed in parallel with the Y direction perpendicular to the incident direction of the radiation 201 (Z direction), for example. In the present exemplary embodiment, as illustrated in FIG. 2, the flexible circuit boards 1140 are situated with the gradient 1141, whereby the area of the flexible circuit boards 1140 in the planar direction (plane including the Y direction) is reduced. As illustrated in FIG. 2, the flexible circuit boards 1140 is provided with the gradient 1141, which enables space saving of the radiographic imaging apparatus 100-1 (for example, thick section 1112) in the planar direction and prevents increase in size. Such an effect increases with the angle of the gradient 1141 of the flexible circuit boards 1140. The greater the difference in height in the Z direction between the radiation detection panel 1130 and the control substrate 1150, the more pronounced the effect. In the present exemplary embodiment, based on the effect, the control substrate 1150 is disposed at a position close to the radiation incident surface 1101, and the radiation detection panel 1130 is disposed at a position close to on the rear surface 1102 of the substrates. However, depending on the configuration, a certain level of effect may be expected even with different arrangements.

The control substrate 1150 is a substrate that controls driving of the radiation detection panel 1130 via the flexible circuit boards 1140. The control substrate 1150 further obtains the image signals related to a radiographic image from the radiation detection panel 1130 via the flexible circuit boards 1140. As illustrated in FIG. 2, this control substrate 1150 is disposed in the thick section 1112. Specifically, as illustrated in FIG. 2, the control substrate 1150 is disposed inside the thick section 1112, at the position close to the side where the radiation 201 is incident relative to the processing substrate 1170.

The wiring 1160 is wiring that connects the control substrate 1150 and the processing substrate 1170. As illustrated in FIG. 2, this wiring 1160 is disposed in the thick section 1112. More specifically, as illustrated in FIG. 2, the wiring 1160 is disposed on the side of the control substrate 1150 and the processing substrate 1170 opposite to a side close to a position where the radiation detection panel 1130 is disposed.

The processing substrate 1170 is a substrate that processes the image signals related to a radiographic image that are signals output from the radiation detection panel 1130. Specifically, the processing substrate 1170 obtains the image signals related to a radiographic image that are output from the radiation detection panel 1130 from the control substrate 1150 via the wiring 1160, and processes the obtained image signals related to a radiographic image. As illustrated in FIG. 2, this processing substrate 1170 is disposed in the thick section 1112.

In the example illustrated in FIG. 2, the control substrate 1150 and the processing substrate 1170 are arranged in this order as seen from the radiation incident surface 1101 of the thick section 1112. Here, as illustrated in FIG. 2, the processing substrate 1170 has a large width in the horizontal direction (Y direction) towards the position where the radiation detection panel 1130 is disposed, compared to the control substrate 1150. With the configuration in which the control substrate 1150 at the position close to the radiation incident surface 1101 of the thick section 1112 is small in width, and the processing substrate 1170 near the radiation detection panel 1130 is large in width, the gradient section 1113 is provided at the border between the thick section 1112 and the thin section 1111. With the gradient section 1113, deformation or fracture due to the concentration of mechanical stress on the border portion between the thick section 1112 and the thin section 1111 is prevented.

As illustrated in FIG. 2, the shielding member 1180 is disposed inside the thick section 1112, between the control substrate 1150 and the processing substrate 1170. The shielding member 1180 is disposed to reduce electromagnetic noise.

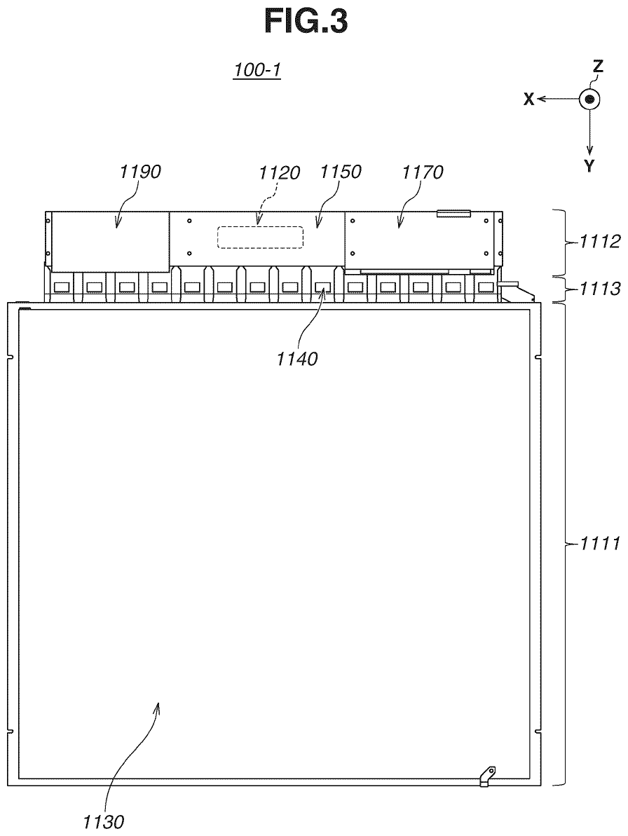

FIG. 3 is a view of the components inside the housing 1110 of the radiographic imaging apparatus 100-1 according to the first exemplary embodiment, seen from the rear surface 1102. In this FIG. 3, components similar to those illustrated in FIGS. 1 and 2 are denoted by the same reference numerals, and a detailed description thereof will be omitted. FIG. 3 also illustrates an XYZ coordinate system corresponding to the XYZ coordinate system illustrated in FIG. 1. Specifically, FIG. 3 is a view of the components inside the housing 1110 of the radiographic imaging apparatus 100-1, seen in the Z direction that is the incident direction of the radiation 201.

As illustrated in FIG. 3, the radiographic imaging apparatus 100-1 further includes a battery 1190 in the thick section 1112 of the housing 1110. This battery 1190 is a power supply that supplies power to the components of the radiographic imaging apparatus 100-1 (such as the radiation detection panel 1130, the flexible circuit boards 1140, the control substrate 1150, and the processing substrate 1170). Examples of the battery 1190 include a lithium-ion battery, an electric double layer capacitor, and an all-solid-state battery. Other batteries may be used.

In FIG. 3, the processing substrate 1170 is illustrated in front of the control substrate 1150 seen from the rear surface 1102, as is also illustrated in FIG. 2. Similarly, in FIG. 3, the battery 1190 is illustrated in front of the control substrate 1150. In the example illustrated in FIG. 3, the control substrate 1150 is disposed across both ends of the thick section 1112 in the X direction. The control substrate 1150 is thus disposed in a long rectangular shape along one side of the radiation detection panel 1130 along the X direction.

As illustrated in FIG. 3, the control substrate 1150 and the processing substrate 1170 are disposed in the thick section 1112 to overlap at least in part as seen in the Z direction that is the incident direction of the radiation 201. The control substrate 1150 and the processing substrate 1170 are thus disposed in the thick section 1112 to overlap as seen in the incident direction of the radiation 201 (Z direction), which reduces the area of the thick section 1112 in the planar direction (XY-plane direction). This enables space saving of the thick section 1112 of the radiographic imaging apparatus 100-1 in the plane direction and prevents increase in size.

As illustrated in FIG. 3, the grip portion 1120 is disposed in the thick section 1112, near the center of one side of the radiation detection panel 1130 along the X direction. In the thick section 1112, the control substrate 1150 and the grip portion 1120 are disposed to overlap at least in part as seen in the Z direction that is the incident direction of the radiation 201. The control substrate 1150 and the grip portion 1120 are thus disposed in the thick section 1112 to overlap as seen in the incident direction of the radiation 201 (Z direction), which reduces the area of the thick section 1112 in the planar direction (XY-plane direction). This enables space saving of the thick section 1112 of the radiographic imaging apparatus 100-1 in the planar direction and prevents increase in size. Specifically, as illustrated in FIG. 2, the positional relationship of the control substrate 1150 and the grip portion 1120 in the Z direction is such that the grip portion 1120 is disposed on a side with the radiation incident surface 1101 and the control substrate 1150 is disposed on a side with the rear surface 1102.

As illustrated in FIG. 3, the control substrate 1150 and the battery 1190 are disposed in the thick section 1112 to overlap at least in part as seen in the Z direction that is the incident direction of the radiation 201. The control substrate 1150 and the battery 1190 are thus disposed in the thick section 1112 to overlap as seen in the Z direction that is the incident direction of the radiation 201 (Z direction), which reduces the area of the thick section 1112 in the planar direction (XY-plane direction). This enables space saving of the thick section 1112 of the radiographic imaging apparatus 100-1 in the planar direction and prevents increase in size.

As illustrated in FIG. 3, the grip portion 1120 and the processing substrate 1170 are disposed in the thick section 1112 without overlapping each other as seen in the Z direction that is the incident direction of the radiation 201. Moreover, as illustrated in FIG. 3, the battery 1190 and the processing substrate 1170 are disposed in the thick section 1112 without overlapping each other as seen in the Z direction that is the incident direction of the radiation 201. Furthermore, as illustrated in FIG. 3, the processing substrate 1170 and the battery 1190 are disposed in the thick section 1112 with the grip portion 1120 therebetween as seen in the Z direction that is the incident direction of the radiation 201.

As illustrated in this FIG. 3, the grip portion 1120, the control substrate 1150, the processing substrate 1170, and the battery 1190 are efficiently arranged in the thick section 1112 as seen in the Z direction that is the incident direction of the radiation 201, whereby the area of the thick section 1112 is reduced.

### Second Exemplary Embodiment

Next, a second exemplary embodiment will be described. In the following description of the second exemplary embodiment, a description of items common to the foregoing first exemplary embodiment is omitted, and differences from the foregoing first exemplary embodiment will be described.

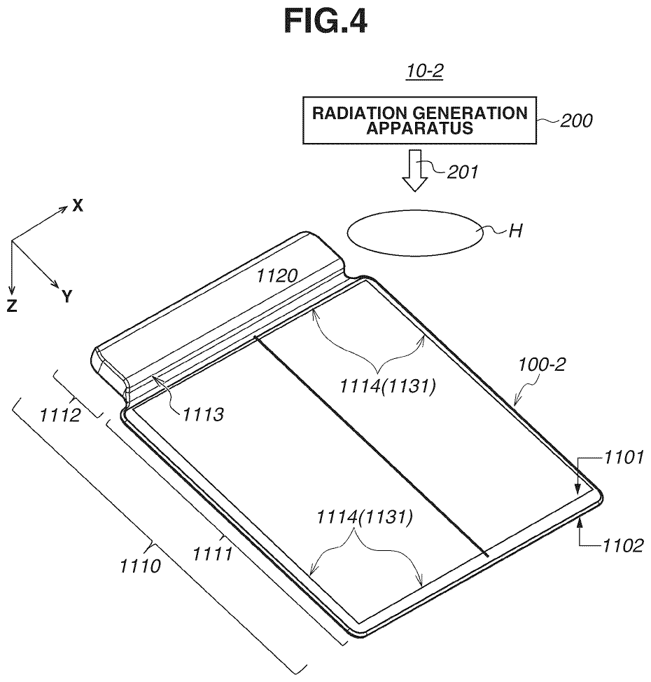

FIG. 4 is a diagram illustrating an example of a schematic configuration of a radiographic imaging system 10-2 according to the second exemplary embodiment. As illustrated in FIG. 4, the radiographic imaging system 10-2 includes a radiographic imaging apparatus 100-2 and a radiation generation apparatus 200. In this FIG. 4, components similar to those illustrated in FIG. 1 are denoted by the same reference numerals, and a detailed description thereof will be omitted. FIG. 4 also illustrates an XYZ coordinate system corresponding to the XYZ coordinate system illustrated in FIG. 1.

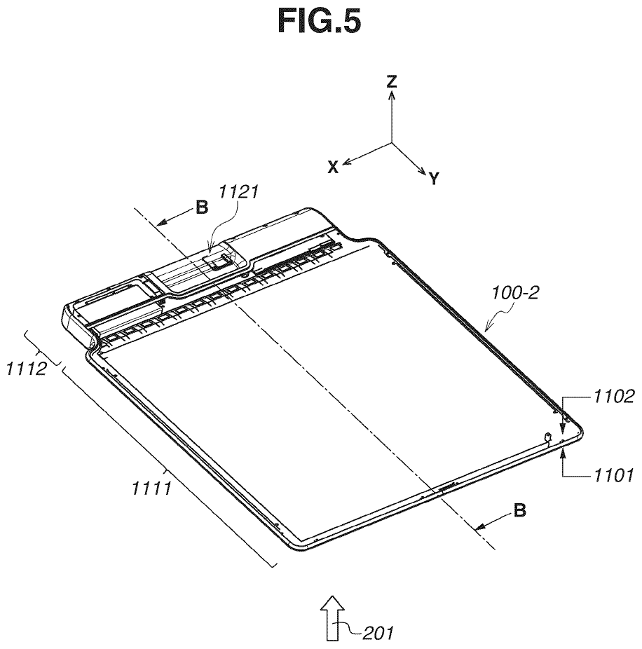

FIG. 5 is a view of the radiographic imaging apparatus 100-2 according to the second exemplary embodiment, seen from the rear surface 1102. In this FIG. 5, component similar to those illustrated in FIGS. 1 and 4 are denoted by the same reference numerals, and a detailed description thereof will be omitted. FIG. 5 also illustrates an XYZ coordinate system corresponding to the XYZ coordinate system illustrated in FIG. 4.

In the radiographic imaging apparatus 100-2 according to the second exemplary embodiment, as illustrated in FIG. 5, a grip portion 1121 for the user to grip the housing 1110 is disposed in the thick section 1112 of the housing 1110 on a side with the rear surface 1102.

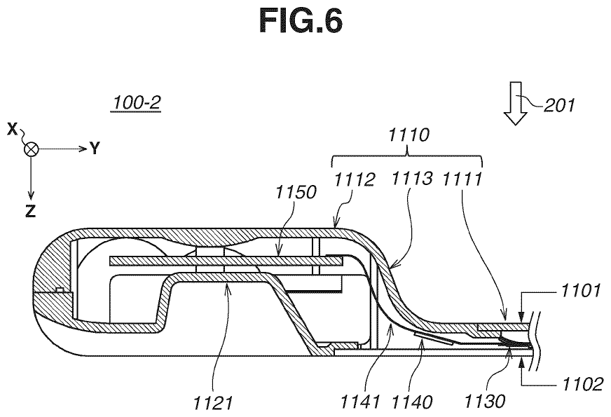

FIG. 6 is a diagram illustrating an example of an internal configuration in cross section B-B of the radiographic imaging apparatus 100-2 according to the second exemplary embodiment illustrated in FIG. 5. In this FIG. 6, components similar to those illustrated in FIGS. 1 to 5 are denoted by the same reference numerals, and a detailed description thereof will be omitted. FIG. 6 also illustrates an XYZ coordinate system corresponding to the XYZ coordinate system illustrated in FIGS. 4 and 5. Specifically, cross section B-B illustrated in FIG. 5 is a cross section along the Y direction.

As illustrated in FIG. 6, the grip portion 1121 is formed in a recessed shape in the thick section 1112 of the housing 1110 on a side with the rear surface 1102 opposite to the radiation incident surface 1101 where the radiation 201 is incident. The grip portion 1121 and the control substrate 1150 are disposed to overlap in part as seen in the Z direction that is the incident direction of the radiation 201. Here, the grip portion 1121 is disposed on a side with the rear surface 1102, and the control substrate 1150 is disposed on a side with the radiation incident surface 1101.

Even in the radiographic imaging apparatus 100-2 according to the second exemplary embodiment, the control substrate 1150 and the processing substrate 1170 are disposed to overlap in part in one side of the thick section 1112. The battery 1190 and the control substrate 1150 are disposed to overlap in part as seen in the incident direction of the radiation 201. Even in the radiographic imaging apparatus 100-2 according to the second exemplary embodiment, the battery 1190 is disposed in an area where neither of the processing substrate 1170 and the grip portion 1121 is disposed as seen in the incident direction of the radiation 201.

Even in the radiographic imaging apparatus 100-2 according to the second exemplary embodiment, like the radiographic imaging apparatus 100-1 according to the first exemplary embodiment, the area of the thick section 1112 in the planar direction (XY-plane direction) is reduced, which prevents increase in size. The grip portion 1120 or 1121 easy for the user to hold may therefore be employed depending on the shape of the thick section 1112. If the thick section 112 has room for accommodation in the thickness direction, a configuration where both the grip portions 1120 and 1121 are disposed may be employed. In such a case, the grip portion 1120, the control substrate 1150, and the grip portion 1121 may be arranged in this order as seen from the radiation incident surface 1101.

### Third Exemplary Embodiment

Next, a third exemplary embodiment will be described. In the following description of the third exemplary embodiment, a description of items common to the foregoing first and second exemplary embodiments is omitted, and differences from the foregoing first and second exemplary embodiments will be described.

In the foregoing first exemplary embodiment, one processing substrate 1170 is disposed in the internal space of the thick section 1112 of the housing 1110. In the third exemplary embodiment, a plurality of processing substrates is disposed.

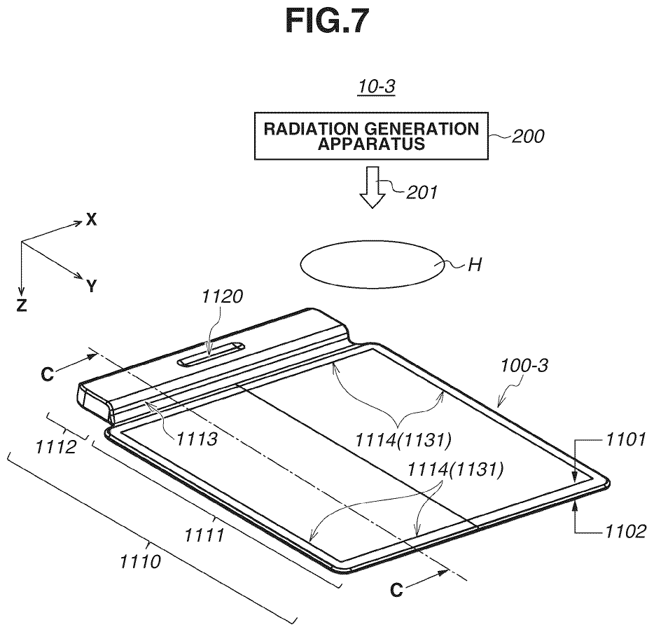

FIG. 7 is a diagram illustrating an example of a schematic configuration of a radiographic imaging system 10-3 according to the third exemplary embodiment. As illustrated in FIG. 7, the radiographic imaging system 10-3 includes a radiographic imaging apparatus 100-3 and a radiation generation apparatus 200. In this FIG. 7, components similar to those illustrated in FIG. 1 are denoted by the same reference numerals, and a detailed description thereof will be omitted. FIG. 7 also illustrates an XYZ coordinate system corresponding to the XYZ coordinate system illustrated in FIG. 1.

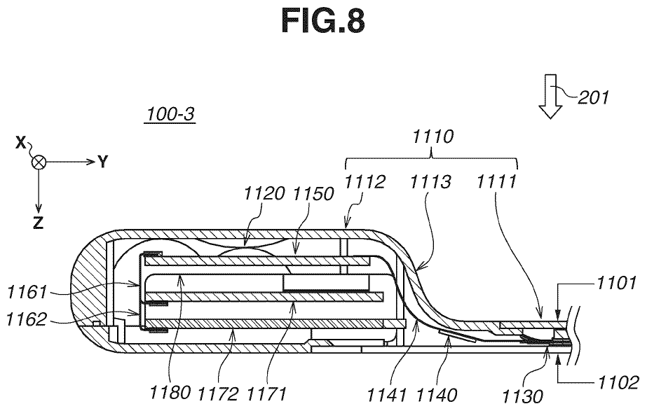

FIG. 8 is a diagram illustrating an example of an internal configuration in cross section C-C of the radiographic imaging apparatus 100-3 according to the third exemplary embodiment illustrated in FIG. 7. In this FIG. 8, components similar to those illustrated in FIGS. 1 to 7 are denoted by the same reference numerals, and a detailed description thereof will be omitted. FIG. 8 also illustrates an XYZ coordinate system corresponding to the XYZ coordinate system illustrated in FIG. 7. Specifically, cross section C-C illustrated in FIG. 7 is a cross section along the Y direction.

The radiographic imaging apparatus 100-3 according to the third exemplary embodiment includes two processing substrates 1171 and 1172 that process image signals related to a radiographic image, or signals output from the radiation detection panel 1130. The radiographic imaging apparatus 100-3 according to the third exemplary embodiment includes the two processing substrate 1171 and 1172 for the sake of distributing functions. For that purpose, the radiographic imaging apparatus 100-3 according to the third exemplary embodiment includes wiring 1161 that connects the control substrate 1150 and the processing substrate 1171, and wiring 1162 that connects the control substrate 1150 and the processing substrate 1172.

In the third exemplary embodiment, the three substrates disposed in the internal space of the thick section 1112, namely, the control substrate 1150 and the processing substrates 1171 and 1172 are disposed to overlap as seen in the Z direction that is the incident direction of radiation 201. While the example illustrated in FIG. 8 includes the two processing substrates 1171 and 1172, three or more processing substrates may be disposed.

To reduce wiring noise between the substrates and the wiring, the third exemplary embodiment employs a positional relationship where the wiring 1161 and 1162 are arranged on one side in the internal space of the thick section 1112 as illustrated in FIG. 8, to prevent occurrence of current loop. Any wiring arrangement may be used as long as the layout does not cause a current loop due to the wiring.

In the example illustrated in FIG. 8, the control substrate 1150, the processing substrate 1171, and the processing substrate 1172 are arranged in this order as seen from the radiation incident surface 1101 of the thick section 1112. Here, as illustrated in FIG. 8, the processing substrate 1172 has a large width in the horizontal direction (Y direction) toward the position where the radiation detection panel 1130 is disposed, compared to the control substrate 1150 and the processing substrate 1171. Moreover, the processing substrate 1171 has a large width in the horizontal direction (Y direction) toward the position where the radiation detection panel 1130 is disposed, compared to the control substrate 1150. With the configuration in which the control substrate 1150 disposed at the position close to the radiation incident surface 1101 of the thick section 1112 is small in width, and the processing substrate 1172 near the radiation detection panel 1130 is large in width, a gradient section 1113 is provided at the border between the thick section 1112 and the thin section 1111. With the gradient section 1113, deformation or fracture due to the concentration of mechanical stress on the border portion between the thick section 1112 and the thin section 1111 is prevented.

Note that all the foregoing first to third exemplary embodiments of the present invention are merely examples of specific implementations for carrying out the present invention, and the technical scope of the present invention should not be interpreted as being limited thereto. In other words, the present invention may be practiced in various forms without departing from the technical concept or essential features thereof.

The first to third exemplary embodiments of the present invention include the following configurations.

### [Configuration 1]

A radiographic imaging apparatus comprising:
a radiation detection panel configured to include an effective imaging area where incident radiation is delectated;
a control substrate configured to control driving of the radiation detection panel;
a processing substrate configured to process a signal output from the radiation detection panel; and
a housing configured to accommodate the radiation detection panel, the control substrate, and the processing substrate,
wherein the housing includes
a first thickness section, having a first thickness in an incident direction of the radiation, where the effective imaging area is disposed, and
a second thickness section, having a second thickness greater than the first thickness in the incident direction of the radiation, where the control substrate and the processing substrate are disposed, and
wherein the control substrate and the processing substrate are disposed to overlap at least in part as seen in the incident direction of the radiation in the second thickness section.

### [Configuration 2]

A radiographic imaging apparatus comprising:
a radiation detection panel configured to include an effective imaging area where incident radiation is detected;
a control substrate configured to control driving of the radiation detection panel;
a housing configured to accommodate the radiation detection panel and the control substrate; and
a grip portion configured to be gripped to hold the housing,
wherein the housing includes
a first thickness section, having a first thickness in an incident direction of the radiation, where the effective imaging area is disposed, and
a second thickness section, having a second thickness greater than the first thickness in the incident direction of the radiation, where the control substrate and the grip portion are disposed, and
wherein the control substrate and the grip portion are disposed to overlap at least in part as seen in the incident direction of the radiation in the second thickness section.

### [Configuration 3]

A radiographic imaging apparatus comprising:
a radiation detection panel configured to include an effective imaging area where incident radiation is detected;
a control substrate configured to control driving of the radiation detection panel;
a flexible circuit board configured to connect the radiation detection panel and the control substrate; and
a housing configured to accommodate the radiation detection panel, the control substrate, and the flexible circuit board,
wherein the housing includes
a first thickness section, having a first thickness in an incident direction of the radiation, where the effective imaging area is disposed,
a second thickness section, having a second thickness greater than the first thickness in the incident direction of the radiation, where the control substrate is disposed, and
a gradient section, connecting the first thickness section and the second thickness section with a gradient, where at least a part of the flexible circuit board is disposed, and
wherein the flexible circuit board connects the radiation detection panel and the control substrate which are disposed at different positions in the incident direction of the radiation, with a gradient.

### [Configuration 4]

The radiographic imaging apparatus according to any one of Configurations 1 to 3, further comprising a battery configured to supply power to the radiographic imaging apparatus, the battery being disposed in the second thickness section of the housing,
wherein the control substrate and the battery are disposed to overlap at least in part as seen in the incident direction of the radiation in the second thickness section.

### [Configuration 5]

The radiographic imaging apparatus according to any one of Configurations 1 to 4, wherein the radiation detection panel and the control substrate are disposed at different positions in the incident direction of the radiation.

### [Configuration 6]

The radiographic imaging apparatus according to any one of Configurations 1 to 5, wherein the second thickness section is thicker than the first thickness section toward a side where the radiation is incident.

### [Configuration 7]

The radiographic imaging apparatus according to Configuration 1, wherein the processing substrate is one or more.

### [Configuration 8]

The radiographic imaging apparatus according to any one of Configurations 1 to 7, wherein the control substrate is disposed at a position close to a side where the radiation is incident, with respect to the control substrate.

### [Configuration 9]

The radiographic imaging apparatus according to Configuration 8, wherein the processing substrate has a large width in a horizontal direction toward a position where the radiation detection panel is disposed, compared to the control substrate.

### [Configuration 10]

The radiographic imaging apparatus according to any one of Configurations 1 and 7 to 9, further comprising a shielding member configured to reduce electromagnetic noise, the shielding member being disposed between the control substrate and the processing substrate.

### [Configuration 11]

The radiographic imaging apparatus according to any one of Configurations 1 and 7 to 10, further comprising a grip portion configured to be gripped to hold the housing, the grip portion being disposed in the second thickness section of the housing,
wherein the grip portion and the processing substrate are disposed without overlapping as seen in the incident direction of the radiation in the second thickness section.

### [Configuration 12]

The radiographic imaging apparatus according to any one of Configurations 1 and 7 to 11, further comprising a battery configured to supply power to the radiographic imaging apparatus, the battery being disposed in the second thickness section of the housing,
wherein the battery and the processing substrate are disposed without overlapping as seen in the incident direction of the radiation in the second thickness section.

### [Configuration 13]

The radiographic imaging apparatus according to any one of Configurations 1 and 7 to 12, further comprising:
a grip portion configured to be gripped to hold the housing, the grip portion being disposed in the second thickness section of the housing; and
a battery configured to supply power to the radiographic imaging apparatus, the battery being disposed in the second thickness section of the housing,
wherein the processing substrate and the battery are disposed with the grip portion therebetween as seen in the incident direction of the radiation in the second thickness section.

### [Configuration 14]

The radiographic imaging apparatus according to any one of Configurations 1 and 7 to 13, further comprising wiring configured to connect the control substrate and the processing substrate,
wherein the wiring is disposed on a side of the control substrate and the processing substrate opposite to a side close to a position where the radiation detection panel is disposed.

### [Configuration 15]

The radiographic imaging apparatus according to Configuration 2, wherein the grip portion is formed in a recessed shape in a side of the second thickness section where the radiation is incident.

### [Configuration 16]

The radiographic imaging apparatus according to Configurations 2 or 15, wherein the grip portion is formed in a recessed shape in a side of the second thickness section opposite to a side where radiation is incident.

### [Configuration 17]

A radiographic imaging system comprising:
the radiographic imaging apparatus according to any one of Configurations 1 to 16; and
a radiation generation apparatus configured to generate the radiation.

According to the foregoing Configurations 1 to 17, increase in thickness of the thick section of the radiographic imaging apparatus is prevented in the planar direction.

### Fourth Exemplary Embodiment

### —Basic Configuration of Radiographic Imaging Apparatus—

Next, a basic configuration of a radiographic imaging apparatus according to a fourth exemplary embodiment will be described.

### [Typical Configuration of Radiographic Imaging Apparatus]

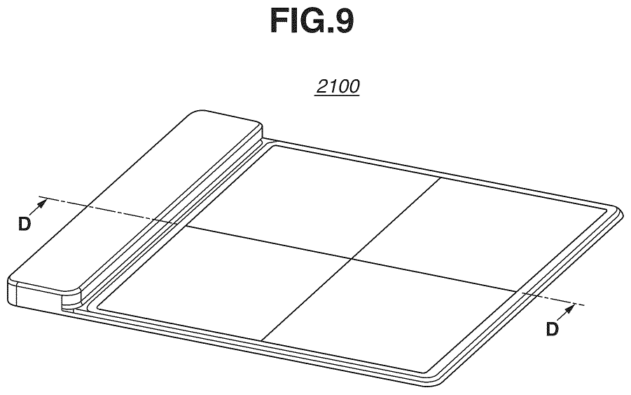

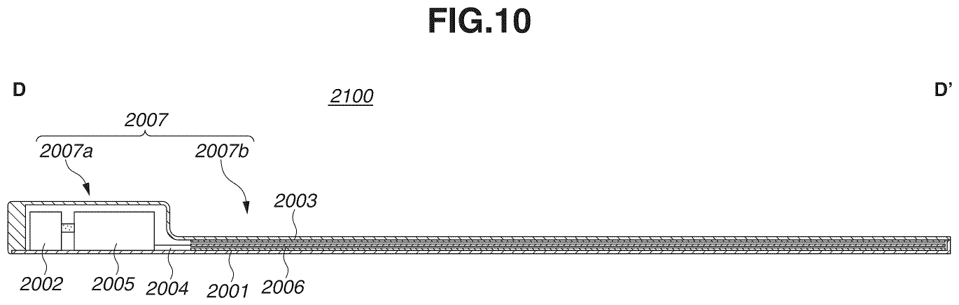

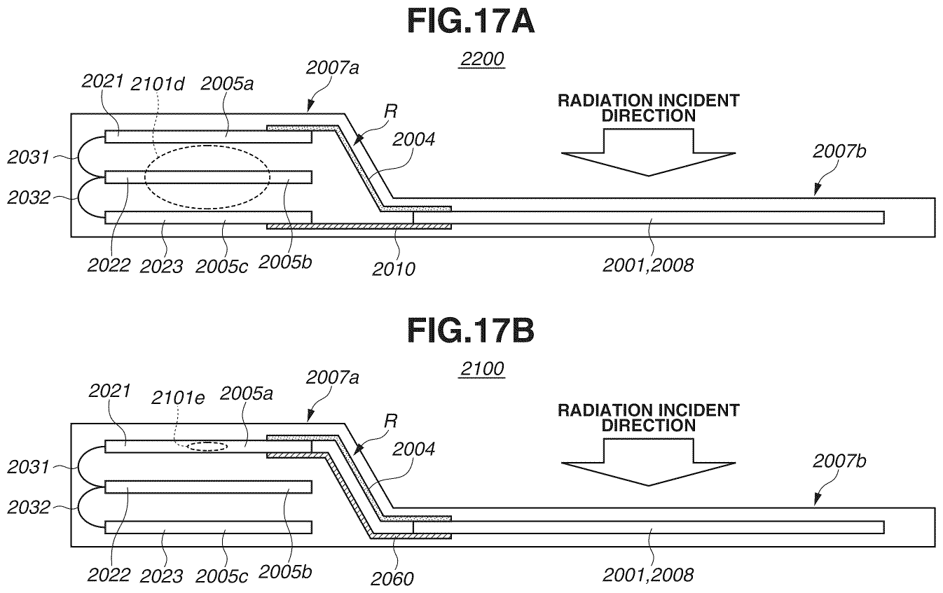

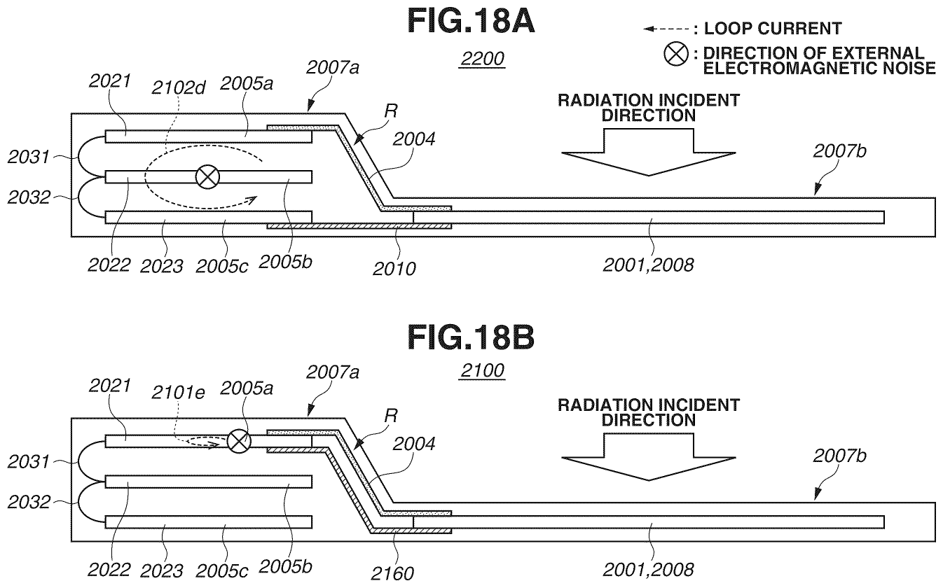

FIG. 9 is a schematic perspective view illustrating the appearance of a typical radiographic imaging apparatus. FIG. 10 is a schematic sectional view taken along dot-dashed line D-D′ in FIG. 9. In FIGS. 9 and 10, a current reduction mechanism of the radiographic imaging apparatus is not illustrated. In this radiographic imaging apparatus, structural members and the like common to those of the radiographic imaging apparatus according to the present exemplary embodiment are denoted by the same reference numerals. In FIGS. 17A, 17B, 18A, and 18B to be described below, a battery 2002, a cushioning member 2003, and a support base 2006 of FIG. 10 are omitted.

A radiographic imaging apparatus 2100 is an apparatus that detects and images radiation emitted from a not-illustrated radiation generation apparatus and transmitted through a subject. The image obtained by the radiographic imaging apparatus 2100 is transferred to outside, displayed on a monitor device or the like, and used for diagnosis etc. The radiographic imaging apparatus 2100 includes a radiation detection panel 2001, signal detection circuits 2004, and a control circuit 2005.

The radiation detection panel 2001 is a radiation detection unit that detects the radiation transmitted through the subject, and includes a sensor substrate on which a large number of photoelectric conversion elements (sensors) are arranged, a phosphor layer (scintillator layer) that is disposed above the sensor substrate, and a phosphor protective film. The radiation detection panel 2001 includes some or all of the plurality of photoelectric conversion elements in its effective imaging area. The effective imaging area is an area that is capable of radiographic imaging and where images are actually generated. In the present exemplary embodiment, the effective imaging area has, but not limited to, a substantially rectangular shape as seen in a radiation incident direction. The phosphor protective film has low moisture permeability and is used to protect the phosphor. The sensor substrate of the radiation detection panel 2001 may be formed of, but not limited to, materials such as glass and flexible plastic.

The radiation detection panel 2001 is connected to the signal detection circuits 2004, and the signal detection circuits 2004 are connected to the control circuit 2005. A battery 2002 for supplying necessary power to the radiographic imaging apparatus 2100 is connected to the control circuit 2005. Examples of the battery 2002 include a lithium-ion battery, an electric double layer capacitor, and an all-solid-state battery. However, the battery 2002 is not limited thereto.

The radiographic imaging apparatus 2100 includes a housing (external casing) 2007 that accommodates the radiation detection panel 2001, the battery 2002, a cushioning member 2003, the signal detection circuits 2004, the control circuit 2005, a support base 2006, and the like. In terms of outer shape, the housing 2007 includes a thick section 2007a that is thick in the radiation incident direction and a thin section 2007b that is thinner than the thick section 2007a. The battery 2002, the control circuit 2005, and the like are disposed in the thick section 2007a. The radiation detection panel 2001, the signal detection circuits 2004, and the like are disposed in the thin section 2007b.

To achieve portability and strength in a compatible manner, the housing 2007 is suitably formed of, but not limited to, magnesium alloys, aluminum alloys, fiber-reinforced plastic, plastic, etc. In particular, the radiation-incident surface of the thin section 2007b where the effective imaging area of the radiation detection panel 2001 is disposed is suitably formed of, but not limited to, a carbon fiber-reinforced plastic or the like with high radiation transmittance and excellent lightweight properties. Moreover, the cushioning member 2003 for protecting the radiation detection panel 2001 from external force and the like is disposed between the radiation detection panel 2001 and the incident surface of the housing 2007. The cushioning member 2003 is suitably formed of, but not limited to, foamed resin, gel, and the like. Moreover, the support base 2006 for supporting the radiation detection panel 2001 is disposed between the radiation detection panel 2001 and the cushioning member 2003. The support base 2006 is suitably formed of, but not limited to, magnesium alloys, aluminum alloys, fiber-reinforced plastic, plastic, and the like with excellent lightweight properties.

When imaging a subject such as a patient, the radiographic imaging apparatus may be placed immediately behind the imaging site of the subject such as a patient. In such a case, due to a step created by the thickness of the radiographic imaging apparatus, the subject such as a patient and the end portion of the radiographic imaging apparatus come into contact to cause a reaction force, and the patient or the like who is the subject may feel discomfort. Typical radiographic imaging apparatuses are often provided in sizes compliant with ISO (International Organization for Standardization) 4090:2001, and often configured with a thickness of approximately 15 mm to 16 mm. In the present exemplary embodiment, since the thin section 2007b of the housing 207 of the radiographic imaging apparatus 2100 has a thickness of 8.0 mm or so, the step created by the radiographic imaging apparatus 2100 during radiographic imaging is small, whereby the reaction force occurring between the subject such as a patient and the end portion of the radiographic imaging apparatus 2100 is reduced. To obtain such an effect, the thickness of the housing of the thin section 2007b does not need to be limited to 8.0 mm or so, and may be even smaller. Specifically, it has been confirmed that the effect is pronounced when the thickness is less than approximately 10.0 mm.

### [Structure of Radiation Detection Panel and Components Nearby]

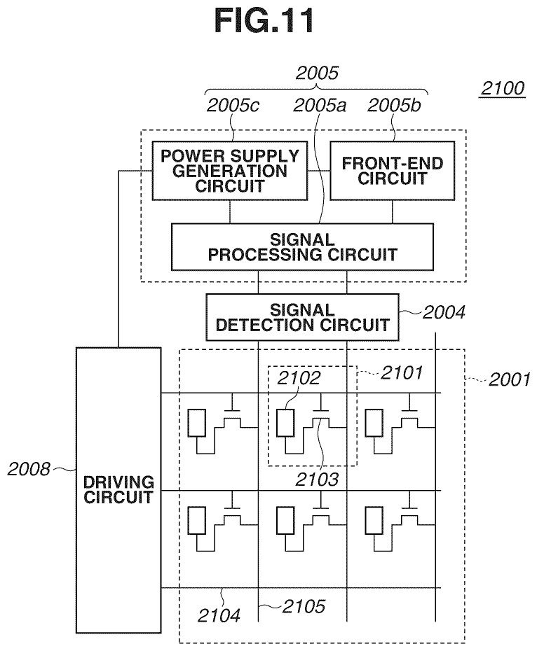

FIG. 11 is a schematic configuration diagram illustrating a typical configuration of the radiographic imaging apparatus.

The radiation detection panel 2001 has a structure where a plurality of pixels 2101 each including a photoelectric conversion element 2102 formed using a semiconductor is arranged in a two-dimensional matrix. Each pixel 2101 includes a photoelectric conversion element 2102 containing amorphous selenium (a-Se) or the like and a switch element 2103 such as a thin-film transistor (TFT), and is covered with a not-illustrated scintillator layer. The scintillator layer is excited based on irradiating radiation and emits visible light. The photoelectric conversion elements 2102 convert the visible light into electrical signals. In other words, the radiation detection panel 2001 is of so-called indirect conversion type that converts the radiation incident via the scintillator layer into electrical signals using the photoelectric conversion elements 2102. The radiation detection panel 2001 is not limited to the indirect conversion type, and may be of so-called direct conversion type where the radiation is directly converted into visible light by the photoelectric conversion elements without the intermediary of the scintillator layer.

The control circuit 2005 electrically connected to the radiation detection panel 2001 via the signal detection circuit 2004 includes a signal processing circuit 2005a and other circuits including a power supply generation circuit 2005c and a front-end circuit 2005b. The signal detection circuit 2004 is a circuit that detects signals output from the radiation detection panel 2001. The signal processing circuit 2005a is a circuit that processes signals output from the signal detection circuit 2004. The front-end circuit 2005b is a circuit including an FPGA, a CPU, or the like, and in charge of various types of processing as a radiographic imaging apparatus. The power supply generation circuit 2005c is a circuit that generates various types of voltages used in the radiographic imaging apparatus.

While the control circuit 2005 here is described to be divided into three types of circuits, there is no limitation on how to divide the control circuit 2005. The three circuits may be integrated into one circuit, or treated as two, four, or more circuits. While FIG. 11 illustrates only one signal detection circuit 2004, the number of signal detection circuits 2004 is not limited. While the one signal detection circuit 2004 is connected with only two signal lines 2105, the number of signal lines 2105 is not limited, either. Analog electrical signals transmitted from the pixels 2101 are detected by the signal detection circuit 2004, and the detected electrical signals are transmitted to the front-end circuit 2005b via the signal processing circuit 2005a.

In driving the radiation detection panel 2001, the front-end circuit 2005b inputs a driving signal to a driving circuit 2008. Moreover, the power supply generation circuit 2005c inputs driving power supply for activating ICs on the driving circuit 2008. In FIG. 11, the driving circuit 2008 is connected to the driving circuit 2008. However, the connection destination may be anywhere within the control circuit 2005. The connection destination may be the front-end circuit 2005b or the signal processing circuit 2005a. The driving circuit 2008 selects a row or column to be driven among the plurality of pixels 2101 constituting the radiation detection panel 2001, based on a control signal received from the front-end circuit 2005b. The driving circuit 208 selects a predetermined row of pixels 2101 via a drive line 2104 using the driving signal. The switch elements 2103 of the pixels 2101 in the selected row turn on sequentially, and image signals (charges) accumulated in the photoelectric conversion elements 2102 of the pixels 2101 in the selected row are output to the signal lines 2105 connected to the respective pixels 2101.

The signal lines 2105 are connected to the control circuit 2005 via the signal detection circuit 2004. The signal detection circuit 2004 includes an amplifier IC and an A/D converter (ADC). The amplifier IC has a function of sequentially reading the image signals output to the signal lines 2105 and amplifying the image signals. The ADC is a unit for converting the analog image signals read by the amplifier IC into digital signals. The digitally converted radiographic image data is input to the control circuit 2005.

### [Occurrence of Loop Currents]

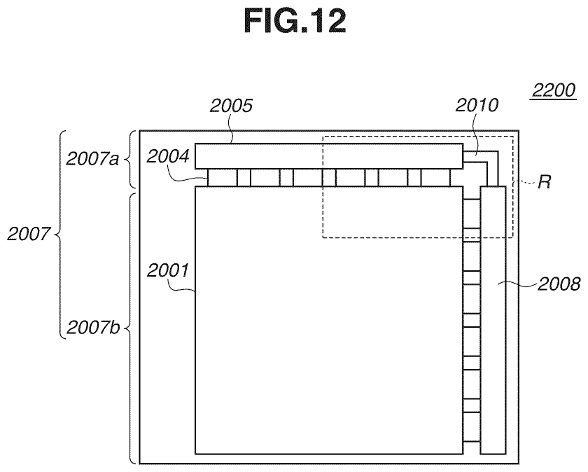

FIG. 12 is a schematic plan view illustrating structural elements of a typical radiographic imaging apparatus as seen from the rear in the radiation incident direction. In FIG. 12, the current reduction mechanism of the radiographic imaging apparatus is not illustrated. In this radiographic imaging apparatus, structural members and the like common to the radiographic imaging apparatus according to the present exemplary embodiment are denoted by the same reference numerals.

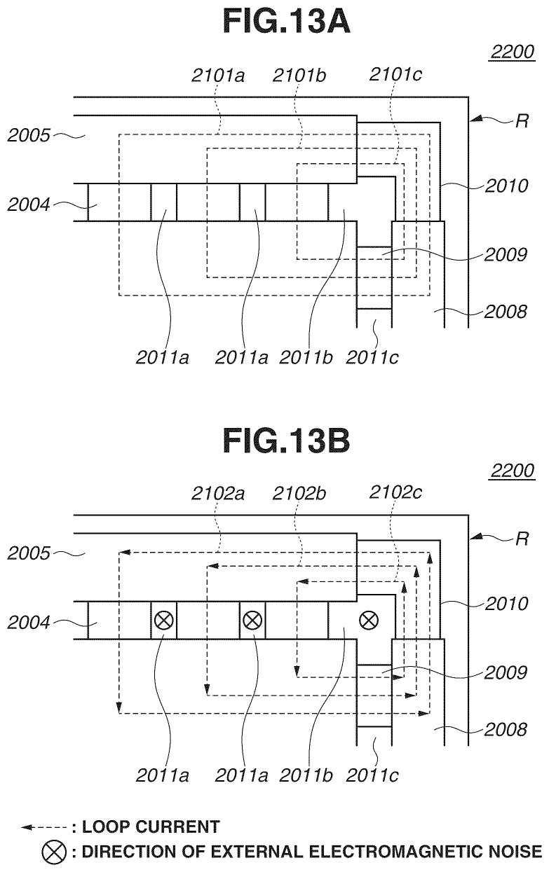

In this radiographic imaging apparatus 2200, a radiation detection panel 2001 is electrically connected to a control circuit 2005 via signal detection circuits 2004, and electrically connected to a driving circuit 2008 via connection wiring (connection lines 2009 of FIGS. 13A and 13B to be described below). The control circuit 2005 and the driving circuit 2008 are electrically connected via a connection line 2010. The control circuit 2005 and the driving circuit 2008 are not folded behind the radiation detection panel 2001 but arranged on the same plane as the radiation detection panel 2001. As a result, depending on the layout and the like of the radiation detection panel 2001 and various circuits, there are entry spots that allow passage of external electromagnetic noise, such as a magnetic field, at predetermined locations. In some radiographic imaging apparatuses, closed circuits of GND loops may be formed between the components of the radiographic imaging apparatuses to surround entry spots for electromagnetic noise. If electromagnetic noise is input to a closed circuit around the entry spot in the radiation detection panel 2001 and passes through the radiographic imaging apparatus 2200, a loop current that causes image noise occurs in the closed circuit of the radiographic imaging apparatus 2200 according to Ampere's law.

FIGS. 13A and 13B are schematic enlarged plan views of the region R surrounded by a broken line in FIG. 12. Specifically, FIG. 13A illustrates a case where electromagnetic noise is not input, and FIG. 13B a case where electromagnetic noise is input. Specifically, the radiographic imaging apparatus 2200 illustrated in FIG. 12 includes three types off gaps 2011a, 2011b, and 2011c illustrated in FIGS. 13A and 13B. The gaps 2011a are formed between adjacent signal detection circuits 2004 vertically sandwiched between the control circuit 2005 and the radiation detection panel 2001. The gap 2011b is formed at a location surrounded by the control circuit 2005, the rightmost signal detection circuit 2004, the radiation detection panel 2001, the topmost connection line 2009, the driving circuit 2008, and the connection line 2010.

The gap 2011c is formed between adjacent connection lines 2009 laterally sandwiched between the radiation detection panel 2001 and the driving circuit 2008. There is no structure capable of electromagnetic shielding in the gaps 2011a, 2011b, or 2011c. The gaps 2011a, 2011b, and 2011c thus function as entry spots for electromagnetic noise.

In a region R, the signal detection circuits 2004, the control circuit 2005, and the driving circuit 2008 have a common ground reference (GND). As illustrated in FIG. 13A, in such a case, closed circuits 2101a, 2101b, and 2101c are formed by GND loops (loops constituted by electrical connection of the driving circuit 2008, the connection line 2010, the control circuit 2005, the signal detection circuits 2004, and the radiation detection panel 2001). The closed circuit 2101a is a loop surrounding the two gaps 2011a and the gap 2011b. The closed circuit 2101b is a loop surrounding one gap 2011a and the gap 2011b. The closed circuit 2101c is a loop surrounding the gap 2011b.

When external electromagnetic noise is input to the two types of gaps 2011a and 2011b in a direction substantially perpendicular to the radiographic imaging apparatus, e.g., in the direction from the rear surface to the front surface, the electromagnetic noise passes through the radiographic imaging apparatus 2200 via each of the two types of gaps 2011a and 2011b. Here, the two types of gaps 2011a and 2011b are disposed in the areas of the respective closed circuits 2101a, 2101b, and 2101c. As a result, according to Ampere's law, loop currents 2102a, 2102b, and 2102c occur in the closed circuits 2101a, 2101b, and 2101c in a direction that counteracts the input electromagnetic noise, i.e., counterclockwise in the example of FIG. 13B. The loop currents 2102a, 2102b, and 2102c cause variations in the amounts of image signals (charges) input to the amplifier IC, and the variations appear as image noise. The greater the area (loop diameter) of a closed circuit, the higher the value of the loop current. The loop current thus becomes higher the farther the input location of the electromagnetic noise from the driving circuit 2008. In the example of FIG. 13B, the loop current 2102a for the largest loop diameter has the highest current value among the loop currents 2102a, 2102b, and 2102c.

The signal detection circuits 2004 are connected with a sensor bias line that provides a reference voltage of the radiation detection panel 2001, and the sensor bias line is affected by the loop currents. An automatic sensing function of performing sensing determination based on the current flowing through the sensor bias line may make a sensing determination with actual radiation irradiation. If radiation is emitted without the user being aware that the radiation detection panel 2001 is determined to have already sensed radiation because of the current, an accidental exposure may result with no image obtained.

While FIG. 13B deals with a case where the electromagnetic noise is input to the radiographic imaging apparatus 2200 substantially perpendicularly in the direction from the rear surface to the front surface of the radiographic imaging apparatus 2200, electromagnetic noise may also be input in a direction from the front surface to the rear surface. In such a case, the loop currents occur in the direction opposite to the foregoing, i.e., clockwise.

### [Current Reduction Mechanism]

As described above, there are gaps that function as entry spots of external electromagnetic nose to the radiographic imaging apparatus. It has been found that loop currents occur in closed circuits due to the electromagnetic noise, and the greater the areas (loop diameters) of the closed circuits occurring in the radiographic imaging apparatus, the higher the loop currents. In the present exemplary embodiment, in view of the foregoing finding, the radiographic imaging apparatus is provided with a current reduction mechanism that reduces loop currents in areas where closed circuits may occur.

Examples of the current reduction mechanism may include the following:
(1) A configuration that precludes loop currents in closed circuits by blocking the input of electromagnetic noise that is the cause of loop currents into entry spots;
(2) A configuration that does not form closed circuits and where no loop current occurs even when electromagnetic noise is input to the radiographic imaging apparatus; and
(3) A configuration with closed circuits of reduced areas, whereby loop currents are suppressed when electromagnetic noise is input to the radiographic imaging apparatus.

For the current reduction mechanism according to the present exemplary embodiment, the configurations that are predicated on the formation of closed circuits and the configuration that does not form closed circuits may be considered. Both shall be included to reduce loop currents in areas where “closed circuits may occur”.

### —First Aspect—

A first aspect of the current reduction mechanism according to the fourth exemplary embodiment will now be described.

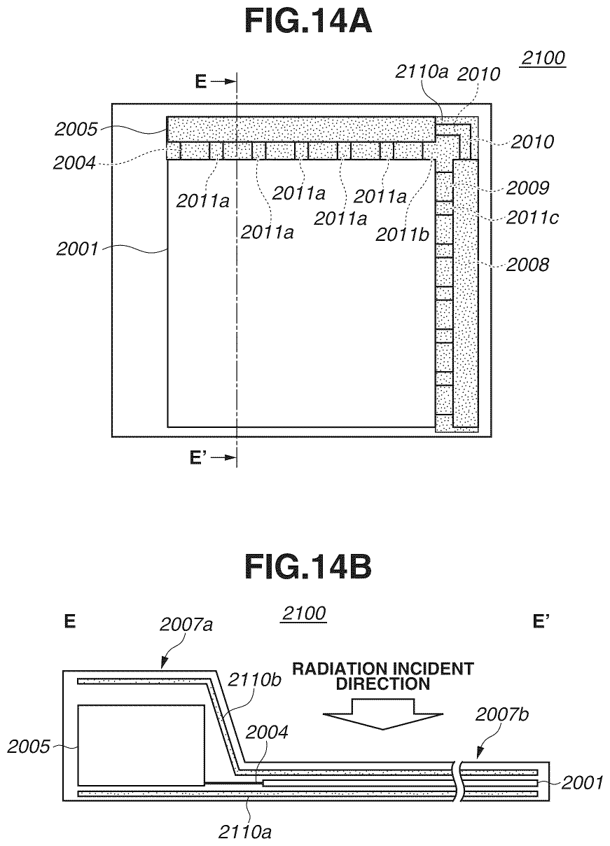

FIGS. 14A and 14B are schematic diagrams illustrating a radiographic imaging apparatus where the current reduction mechanism according to the first aspect is disposed. Specifically, FIG. 14A is a schematic plan view of the radiographic imaging apparatus seen from the rear. FIG. 14B is a schematic cross-sectional view along dot-dashed line E-E′ of FIG. 14A.

The current reduction mechanism according to the first aspect is a specific implementation of the foregoing configuration (1), and includes electromagnetic shields disposed to cover the entry spots of electromagnetic noise. The electromagnetic shields are sheet-like members covering at least a part of the area where closed circuits of GND loops are formed, and are formed of materials such as magnetic materials and plastic. For example, an electromagnetic shield formed by laminating a PET or other plastic film on the surface of a permalloy or other magnetic material sheet is suitably used. In the first aspect, electromagnetic shields 2110a and 2110b are disposed on the rear and front sides in the housing 2007 so that the signal detection circuits 2004, the control circuit 2005, the driving circuit 2008, and the connection line 2010 are all covered, including the gaps 2011a, 2011b, and 2011c. Here, since the front surface of the radiation detection panel 2001 serves as the radiation incident surface, the electromagnetic shields 2110a and 2110b desirably do not overlap the radiation detection panel 2001 in a plan view.

With the electromagnetic shields 2110a and 2110b provided in the radiographic imaging apparatus 2100, the gaps 2011a, 2011b, and 2011c are closed off with the electromagnetic shields 2110a and 2110b. This blocks the input of electromagnetic noise into the gaps 2011a, 2011b, and 2011c. The occurrence of loop currents in the closed circuits due to the external electromagnetic noise is thereby prevented. In the first aspect, the electromagnetic shields are disposed on both the front and rear sides in the housing 2007. The input to the gaps 2011a, 2011b, and 2011c is thus blocked regardless of which side the external electromagnetic noise is incident from, the front side or the rear side. The radiographic imaging apparatus 2100 is therefore not affected by external magnetic noise, and the occurrence of loop currents is prevented as much as possible. The effect of reducing loop currents is also obtainable if an electromagnetic shield is disposed only on the front surface that is the radiation incident surface, for example.

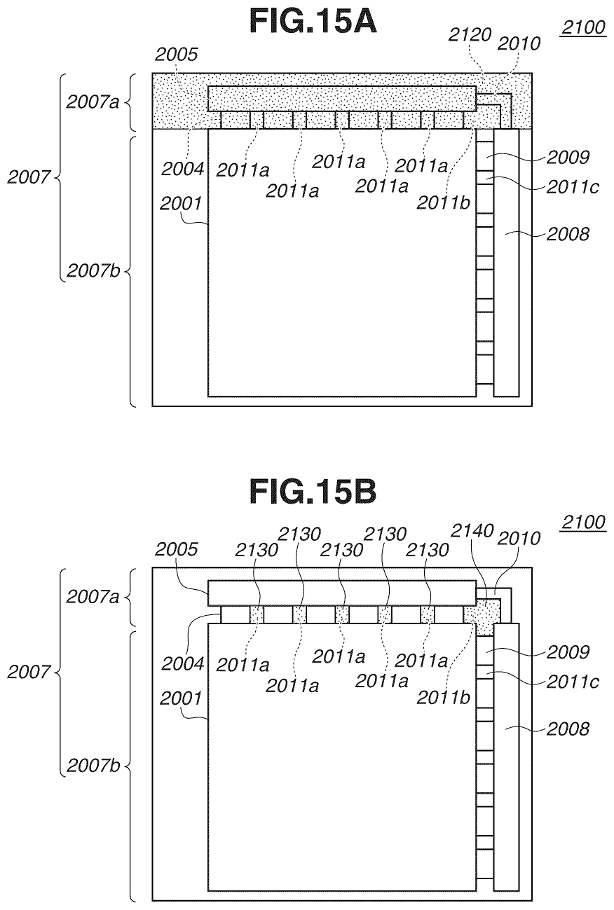

The current reduction mechanism according to the first aspect is not limited to the foregoing electromagnetic shields 2110a and 2110b. FIGS. 15A and 15B are schematic diagrams illustrating radiographic imaging apparatuses provided with current reduction mechanisms according to other examples of the first aspect of the fourth exemplary embodiment.

FIG. 15A illustrates a first example of the electromagnetic shields. It has been found that the signal detection circuits 2004 account for most of the effect of loop currents among the components of the radiographic imaging apparatus 2100. The signal detection circuits 2004 not only cause loop currents but may also cause noise inside due to the input of electromagnetic noise into the signal detection circuits 2004. A current reduction mechanism is thus provided to cover the signal detection circuits 2004 as well as entry spots of electromagnetic noise that causes loop currents in the closed circuits of GND loops including the signal detection circuits 2004. This prevents most of the effect of loop currents as well as the effect of electromagnetic noise input to the signal detection circuits 2004. In the first example, in view of the foregoing findings, electromagnetic shields 2120 are provided to cover closed circuits of GND loops including the signal detection circuits 2004 that are highly affected by electromagnetic noise when the closed circuits including the signal detection circuits 2004 are formed. The electromagnetic shield 2120 are disposed on both the front and rear sides in the housing 2007 so that the upper end portion including the signal detection circuits 2004, the control circuit 2005, the connection line 2010, and the gaps 2011a and 2011b is covered.

With the electromagnetic shields 2120 in the radiographic imaging apparatus 2100, the volume of the current reduction mechanism added to the radiographic imaging apparatus is reduced and most of the effect of loop currents are efficiently eliminated by preventing the occurrence of loop currents.

FIG. 15B illustrates a second example of the electromagnetic shields. The second example deals with an aspect where the concept of the first example is further advanced, considering cases where electromagnetic noise will not be input to the signal detection circuits 2004 or where electromagnetic noise is input to the signal detection circuits 2004 but without much effect. In the second example, electromagnetic shields 2130 and 2140 are disposed on the front and rear sides in the housing 2007, respectively, so that only the gaps 2011a and 2011b are covered. Such a configuration further reduces the volume of the current reduction mechanism added to the radiographic imaging apparatus and more efficiently eliminates most of the effect of loop currents by preventing the occurrence of loop currents. To reduce the effect of the loop currents with higher reliability, electromagnetic shields covering the gaps 2011c may be disposed in addition to the configuration of FIG. 15A or the configuration of FIG. 15B.

### —Second Aspect—

A second aspect of the current reduction mechanism according to the fourth exemplary embodiment will now be described.

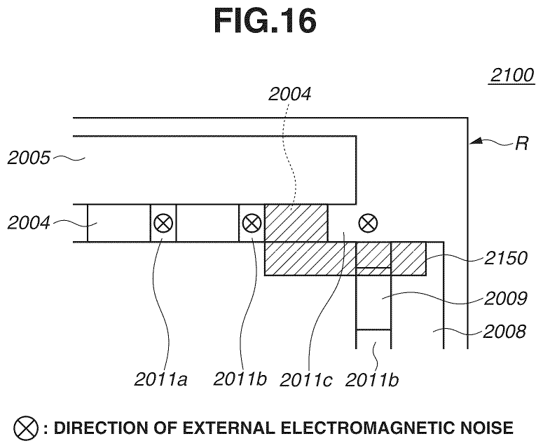

FIG. 16 is a schematic enlarged plan view of the region R where the current reduction mechanism according to the second aspect is disposed in the radiographic imaging apparatus according to the fourth exemplary embodiment.

As described above, it has been found that the signal detection circuits 2004 account for most of the effect of loop currents among the components of the radiographic imaging apparatus 2100. The second aspect is a specific implementation of the foregoing configuration (2), where a current reduction mechanism is disposed in an area where the presence of closed circuits of GND loops including the signal detection circuits 2004 causes issues. The current reduction mechanism according to the second aspect includes an electrical connection member that is laid along a wiring route that does not form a closed circuit among a plurality of wiring routes selectable in that area. This electrical connection member is a connection line 2150 that is disposed to overlap a signal detection circuit 2004 at least in part when seen in a plan view and electrically connects the control circuit 2005 and the driving circuit 2008.

In a radiographic imaging apparatus, for example, as illustrated in FIG. 12, the connection line 2010 is typically disposed as the electrical connection member electrically connecting the control circuit 2005 and the driving circuit 2008, using the space at the top right end of the radiographic imaging apparatus. In such a case, however, as illustrated in FIGS. 13A and 13B, the closed circuits 2101a, 2101b, and 2101c of GND loops are formed, and the loop currents 2102a, 2101b, and 2102c occur due to the input of external electromagnetic noise. In the second aspect, focusing on the electrical connection configuration between the control circuit 2005 and the driving circuit 2008, a plurality of wiring routes selectable to connect the two circuits in the region R was searched for a wiring route that does not form the closed circuits 2101a, 2101b, or 2101c. As a result, the wiring route overlapping the signal detection circuits 2004 at least in part when seen in a plan view was found. The connection line 2150 laid along this wiring route is connected to the control circuit 2005 at one end, passes over the rightmost signal detection circuit 2004 and a part of the radiation detection panel 2001, and is connected to the driving circuit 2008 at the other end. Here, the connection line 2150 is desirably disposed to avoid the effective pixel area and overlap a part of the radiation detection panel 2001 outside the effective pixel area when seen in a plan view, so that the incidence of radiation on the photoelectrical conversion elements in the effective pixels (pixels actually used for imaging) is not interfered.

As the connection line 2150, an FFC (flat flexible cable), an FPC (flexible printed circuit), or an FFC or FPC covered with a noise reduction member such as a magnetic material is used. Alternatively, an electric wire covered with a vinyl or other insulating coating may be used.

In FIGS. 13A and 13B, the connection line 2010 constitutes a part of the closed circuits 2101a, 2101b, and 2101c. Without the connection line 2010, the GND loops are disconnected there, no closed circuit is formed in the region R, and no loop current. In the second aspect, as illustrated in FIG. 16, the connection line 2150 is disposed instead of the connection line 2010, whereby the control circuit 2005 and the driving circuit 2008 are electrically connected without forming a closed circuit. Here, the incidence of electromagnetic noise on the gaps 2011a and 2011b does not cause a loop current, since there is no closed circuit surrounding the gaps 2011a or 2011b.

If the ordinary connection line 2010 is used to electrically connect the control circuit 2005 and the driving circuit 2008 as illustrated in FIGS. 13A and 13B, the signal detection circuit 2004 is exposed in the housing 2007. External electromagnetic noise may therefore be incident not only on the gaps 2011a, 2011b, and 2011c, but on the signal detection circuit 2004 as well. This electromagnetic noise may then produce noise in the signal detection circuit 2004. In the second exemplary embodiment, the connection line 2150 is disposed to overlap the rightmost signal detection circuit 2004. As a result, the connection line 2150 blocks the external electromagnetic noise, whereby the incidence of the electromagnetic noise on the signal detection circuit 2004 is prevented and the occurrence of noise in the signal detection circuit 2004 is suppressed. Here, for example, the use of an FFC, FPC, or the like covered with a noise reduction member as the connection line 2150 prevents the incidence of electromagnetic noise into the signal detection circuit 2004 with higher reliability.

When the connection line 2150 is provided instead of the connection line 2010, the connection line 2150 is disposed to overlap the signal detection circuit 2004 and a part of the radiation detection panel 2001. This increases the thickness of the thick section 2007a of the housing 2007 as compared to the case where the connection line 2010 is used. The thick section 2007a includes many structures and tends to undergo force due to warping of the radiation detection panel 2001 when the user (operator) grips the thick section 2007a and carries the radiographic imaging apparatus. With the connection line 2150 instead of the connection line 2010, the thickness of the thick section 2007a is increased, and the strength of the radiographic imaging apparatus 2100 is improved. In such a manner, the second aspect improves the workability (usability) of the user of the radiographic imaging apparatus 2100.

### —Third Aspect—

A third aspect of the current reduction mechanism according to the fourth exemplary embodiment will now be described.

FIGS. 17A and 17B are schematic diagrams illustrating the current reduction mechanism according to the third aspect along with a typical radiographic imaging apparatus, illustrating a state where a closed circuit is formed in the radiographic imaging apparatus of the fourth exemplary embodiment.

FIG. 17A is a schematic cross-sectional view illustrating the typical radiographic imaging apparatus. FIG. 17B is a schematic cross-sectional view illustrating the third aspect. FIGS. 18A and 18B are schematic diagrams illustrating the current reduction mechanism according to the third aspect along with a typical radiographic imaging apparatus, illustrating a state where a loop current occurs in the radiographic imaging apparatus of the fourth exemplary embodiment. FIG. 18A is a schematic cross-sectional view illustrating the typical radiographic imaging apparatus. FIG. 18B is a schematic cross-sectional view illustrating the third aspect.

In the radiographic imaging apparatuses 2100 and 2200, the control circuit 2005 includes a plurality of circuit substrates tacked on each other. Specifically, as illustrated in FIGS. 17A and 17B, for example, a first substrate 2021, a second substrate 2022, and a third substrate 2023 are stacked on each other at predetermined distances in the thick section 2007a of the housing 2007. The first substrate 2021 is a circuit substrate that includes the signal processing circuit 2005a and with which the signal detection circuits 2004 are in contact in part, whereby the signal processing circuit 2005a is electrically connected to the signal detection circuits 2004. The first substrate 2021 is disposed in the upper section. The second substrate 2022 is a circuit substrate including the front-end circuit 2005b electrically connected to the signal processing circuit 2005a by wiring 2031, and is disposed in the center section. The third substrate 2023 is a circuit substrate that includes the power supply generation circuit 2005c electrically connected to the front-end circuit 2005b by wiring 2032, and is disposed in the lower section. In FIGS. 17A and 17B, the first substrate 2021 (signal processing circuit 2005a), the second substrate 2022 (front-end circuit 2005b), and the third substrate 2023 (power supply generation circuit 2005c) are arranged in this order from the radiation incident side. However, such order is not restrictive. The number of circuit substrates stacked is not limited to three as described above. Two, four, or more layers may be stacked.

As illustrated in FIGS. 17A, since the control circuit 2005 has the layered structure including a plurality of circuit substrates, a large DNG loop is formed in the region R including the side portion of the control circuit 2005. This GND loop forms a closed circuit 2101d that connects the driving circuit 2008, the connection line 2010, the control circuit 2005 (the power supply generation circuit 2005c, the wiring 2032, the front-end circuit 2005b, the wiring 2031, and the signal processing circuit 2005a), the signal detection circuits 2004, and the radiation detection panel 2001. In the region R, for example, the side surface of the second substrate 2022 including the front-end circuit 2005b functions as a potential entry point that allows passage of external electromagnetic noise such as a magnetic field. As illustrated in FIG. 18A, if electromagnetic noise is input to the entry spot and passes through the front-end circuit 2005b, a loop current 2102d that causes image noise occurs in the closed circuit 2101d. The magnitude of the loop current depends on the area (or loop diameter) of the closed circuit where the loop current occurs. Since the closed circuit 2101d has a large loop diameter corresponding to the thickness of the control circuit 2005, the loop current 2102d also has a large value.

The current reduction mechanism according to the third aspect is a specific implementation of the foregoing configuration (3), and includes an electrical connection member that is laid along a wiring route of the smallest closed-circuit area among those corresponding to a plurality of wiring routes selectable in the region R. According to the third aspect, the signal detection circuits 2004 are brought into contact with and electrically connected to one of the front and rear surfaces of a circuit substrate that is one of the first, second, and third substrates 2021, 2022, and 2023. The foregoing electrical connection member refers to a connection line 2160 that is in contact with the other of the front and rear surfaces of the circuit substrate to which the signal detection circuits 2004 are connected. The first substrate 2021, the second substrate 2022, and the third substrate 2023 are electrically connected by wiring 2031 and 2032, and the control circuit 2005 is thereby effectively connected to the signal detection circuits 2004 and the connection line 2160. The third aspect will now be described by using a configuration where the signal detection circuits 2004 and the connection line 2160 are electrically connected to the signal processing circuit 2005a through contact with the front surface and the rear surface of the first substrate 2021 of the control circuit 2005 as an example.

As illustrated in FIG. 17A, a radiographic imaging apparatus typically includes the connection line 2010 as an electrical connection member that electrically connects the driving circuit 2008 and the control circuit 2005. Among the first, second, and third substrates 2021, 2022, and 2023 constituting the control circuit 2005, the third substrate 2023 is disposed on substantially the same plane as and closest to the driving circuit 2008. The connection line 2010 is thus brought into contact with the third substrate 2023 to electrically connect the driving circuit 2008 and the power supply generation circuit 2005c. However, in such a case, the large closed circuit 2101d is formed to cause the loop current 2102d as described above. If the signal detection circuits 2004 are brought into contact with and electrically connected to the first substrate 2021 and the connection line 2010 is brought into contact with and electrically connected to the second substrate 2022, a closed circuit that is smaller than the closed circuit 2101d but relatively large with a loop diameter as much as two layers of circuit substrates is formed.

In the third aspect, as illustrated in FIG. 17B, focusing on the electrical connection between the control circuit 2005 and the driving circuit 2008, a plurality of wiring routes selectable to connect the two circuits in the region R was searched for a wiring route where the closed-circuit area is the smallest among those corresponding to the plurality of wiring routes. As a result, the connection line 2160 was found. Like the connection of the signal detection circuits 2004, the connection line 2160 is brought into contact with the first substrate 2021 to electrically connect the driving circuit 2008 and the signal processing circuit 2005a. Specifically, the signal detection circuits 2004 are connected to one of the front and rear surfaces (for example, the front surface) of the first substrate 2021, and the connection line 2160 is connected to the other of the front and rear surfaces (for example, the rear surface) of the first substrate 2021. This forms a closed circuit 2101e inside the first substrate 2021. In the region R, for example, the side surface of the first substrate 2021 including the signal processing circuit 2005a functions as an entry spot that allows the passage of external electromagnetic noise such as a magnetic field. As illustrated in FIG. 18B, if electromagnetic noise is input to this entry spot and passes through the signal processing circuit 2005a, a loop current 2102e occurs in the closed circuit 2101e. However, the closed circuit 2101e has the smallest size with the loop diameter equivalent to the thickness of the first substrate 2021 among the closed circuits that occur in the region R. The value of the loop current 2102e occurring in the closed circuit 2101e is therefore also the smallest. Since the loop current 2102e occurs in the closed circuit 2101e with an extremely small loop diameter of, e.g., approximately 1 mm that is the thickness of the first substrate 2021, the amount of occurrence thereof is almost negligibly small. As described above, in the third aspect, image noise and unexpected abnormal operation occurring due to loop currents are reduced as much as possible by minimizing the amount of occurrence of loop currents in the control circuit 2005.

As the connection line 2160, like the connection line 2150 described in the second aspect, an FFC, an FPC, or an FFC or FPC covered with a noise reduction member such as a magnetic material is used. Alternatively, an electric wire covered with a vinyl or other insulating coating may be used.

In the control circuit 2005, as illustrated in FIG. 17B, the first substrate 2021 and the second substrate 2022 are desirably electrically connected by the wiring 2031 at only one side, and the second substrate 2022 and the third substrate 2023 by the wiring 2032 at only one side. It is undesirable to electrically connect the circuit substrates at both sides, since such a connection forms closed circuits. In the radiographic imaging apparatus 2100 according to the third aspect, as illustrated in FIG. 17B, the signal detection circuits 2004 and the connection line 2160 are disposed substantially in parallel, and the distance therebetween is desirably less than or equal to the thickness of the third substrate 2023, such as 1 mm or less.

As described above, according to various aspects of the radiographic imaging apparatus of the fourth exemplary embodiment, the occurrence of loop currents due to external electromagnetic noise is reduced to suppress image noise and unexpected abnormal operation by simple techniques.

### Fifth Exemplary Embodiment

### —Basic Configuration of Radiographic Imaging Apparatus—

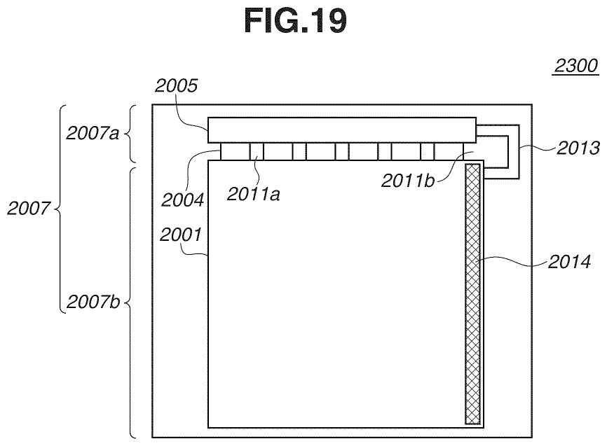

FIG. 19 is a schematic plan view of a typical configuration of a radiographic imaging apparatus according to a fifth exemplary embodiment, seen from a rear side in a radiation incident direction. In FIG. 19, a current reduction mechanism of the radiographic imaging apparatus is not illustrated. In this radiographic imaging apparatus, structural members and the like common to the radiographic imaging apparatus according to the fourth exemplary embodiment are denoted by the same reference numerals.

The radiographic imaging apparatus according to the fifth exemplary embodiment is an apparatus including a so-called WOA (Wire on Array) radiation detection panel. A radiographic imaging apparatus 2300 includes a radiation detection panel 2001, signal detection circuits 2004, and a control circuit 2005. In the fifth exemplary embodiment, the radiation detection panel 2001 is configured as a WOA type, and a drive line 2014 is disposed inside the radiation detection panel 2001 instead of the driving circuit 2008 of FIG. 12. The radiation detection panel 2001 is connected to the control circuit 2005 by a connection line 2013 corresponding to the connection line 2010 of FIG. 12, whereby the control circuit 2005 and the drive line 2014 are electrically connected.

In the radiographic imaging apparatus 2300, like the radiographic imaging apparatus 2200 of FIG. 12, gaps 2011a and 2011b function as entry spots of external electromagnetic noise. Since the radiation detection panel 2001 is of WOA type, the radiographic imaging apparatus 2300 does not have the gaps 2011c of FIG. 13A or 13B. If electromagnetic noise passes through the gaps 2011a and 2011b and through the radiographic imaging apparatus 2300, loop currents occur in closed circuit as in FIGS. 13A and 13B.

### —First Aspect—

A first aspect of the current reduction mechanism according to the fifth exemplary embodiment will be described.

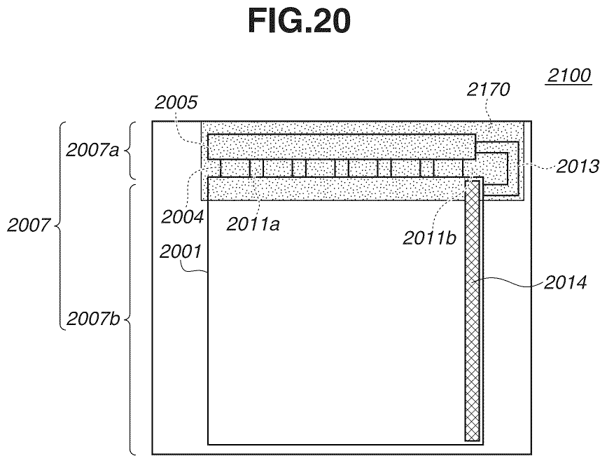

FIG. 20 is a schematic plan view illustrating the radiographic imaging apparatus including the current reduction mechanism according to the first aspect of the fifth exemplary embodiment.

In the first aspect, like the first aspect of the fourth exemplary embodiment, electromagnetic shields 2170 are disposed as the current reduction mechanism on the front and rear sides in the housing 2007 so that the radiation detection panel 2001, the signal detection circuits 2004, the control circuit 2005, and the connection line 2013 are covered, including the gaps 2011a and 2011b. With the electromagnetic shields 2170 provided in the radiographic imaging apparatus 2100, the gaps 2011a and 2011b are closed off with the electromagnetic shields 2170. This blocks the input of electromagnetic noise to the gaps 2011a and 2011b. The occurrence of loop currents in the closed circuits due to external electromagnetic noise is thereby prevented.

### —Second Aspect—

A second aspect of the current reduction mechanism according to the fifth exemplary embodiment will now be described.

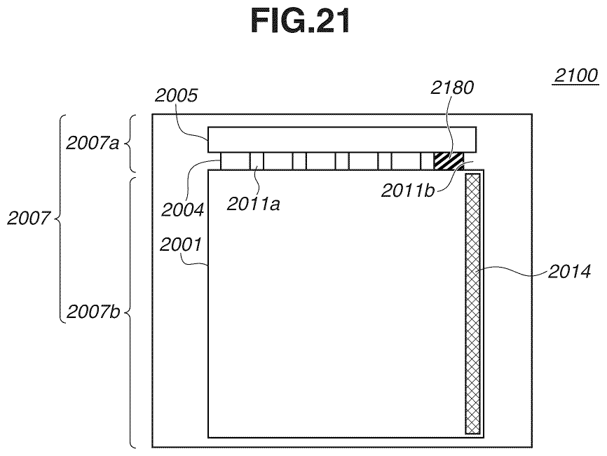

FIG. 21 is a schematic plan view illustrating a radiographic imaging apparatus including the current reduction mechanism according to the second aspect of the fifth exemplary embodiment.

In the second aspect, like the second aspect of the fourth exemplary embodiment, a connection line 2180 is disposed as the current reduction mechanism instead of the connection line 2013 that forms closed circuits. The connection line 2180 is connected to the control circuit 2005 at one end, passes over the rightmost signal detection circuit 2004, and is connected to the radiation detection panel 2001 at the other end. The control circuit 2005 and the drive line 2014 are thereby electrically connected.

In FIG. 19, the connection line 2013 forms a part of closed circuits. Without the connection line 2013, the GND loops are disconnected there, no closed circuit is formed, and no loop current occurs. In the second aspect, as illustrated in FIG. 21, the connection line 2180 is disposed instead of the connection line 2013, whereby the control circuit 2005 and the drive line 2014 are electrically connected without forming a closed circuit. In such a case, even if electromagnetic noise enters the gaps 2011a and 2011b, no loop current occurs since there is no closed circuit surrounding the gaps 2011a or 2011b. Moreover, since the rightmost signal detection circuit 2004 is covered by the connection line 2180, the input of electromagnetic noise to the signal detection circuit 2004 is reduced by the connection line 2180, whereby the occurrence of a loop current in the signal detection circuit 2004 is suppressed.

The radiation detection panel 2001 is configured as a WOA type, including the drive line 2014 inside. Since the driving circuit is omitted, it is sufficient for the connection line 2180 to have enough length to cover the signal detection circuit 2004. Thus, the connection line 2180 is configured short, which results in a significant reduction in cost.

In the fifth exemplary embodiment, like the third aspect of the fourth exemplary embodiment, if the control circuit 2005 includes a stack of a plurality of circuit substrates, the signal detection circuits 2004 may be connected to the front surface of the first substrate 2021 and the connection line that is the current reduction mechanism may be connected to the rear surface. This minimizes the amount of occurrence of loop currents in the control circuit 2005.

As described above, according to various aspects of the radiographic imaging apparatus of the fifth exemplary embodiment, the occurrence of loop currents due to external electromagnetic noise is reduced to suppress image noise and unexpected abnormal operation by simple techniques.

### Sixth Exemplary Embodiment

### —Basic Configuration of Radiographic Imaging Apparatus—

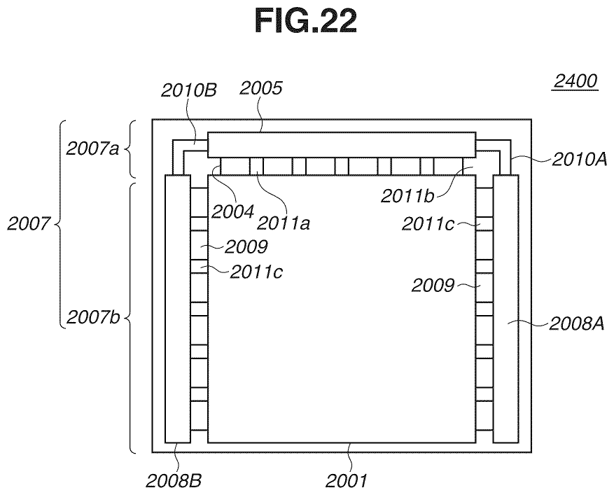

FIG. 22 is a schematic plan view of a typical configuration of a radiographic imaging apparatus according to a sixth exemplary embodiment, seen from a rear side in a radiation incident direction. In FIG. 22, a current reduction mechanism of the radiographic imaging apparatus is not illustrated. In this radiographic imaging apparatus, structural members and the like common to the radiographic imaging apparatus according to the fourth exemplary embodiment are denoted by the same reference numerals.

The radiographic imaging apparatus according to the sixth exemplary embodiment includes at least two or more driving circuits. An example of a radiographic imaging apparatus of so-called dual readout type, where driving circuits are disposed on both sides of a radiation detection panel 2001, will now be described. A radiographic imaging apparatus 2400 includes the radiation detection panel 2001, signal detection circuits 2004, a control circuit 2005, and driving circuits 2008A and 2008B. The driving circuits 2008A and 2008B are connected to the right and left sides of the radiation detection panel 2001, respectively, so that the radiation detection panel 2001 is sandwiched therebetween in FIG. 22. This corresponds to a case where the driving circuit 2008 according to the fourth exemplary embodiment is divided in two or where another driving circuit 2008 is added. The driving circuit 2008A is connected to the control circuit 2005 via a connection line 2010A. The driving circuit 2008B is electrically connected to the control circuit 2005 via a connection line 2010B.

In the radiographic imaging apparatus 2400, like the radiographic imaging apparatus 2200 of FIG. 12, the gaps 2011a, 2011b, and 2011c function as entry spots of external electromagnetic noise. If electromagnetic noise passes through the gaps 2011a and 2011b and through the radiographic imaging apparatus 2400, loop currents occur in the closed circuits as in FIGS. 13A and 13B.

### —First Aspect—

A first aspect of the current reduction mechanism according to the sixth exemplary embodiment will now be described.

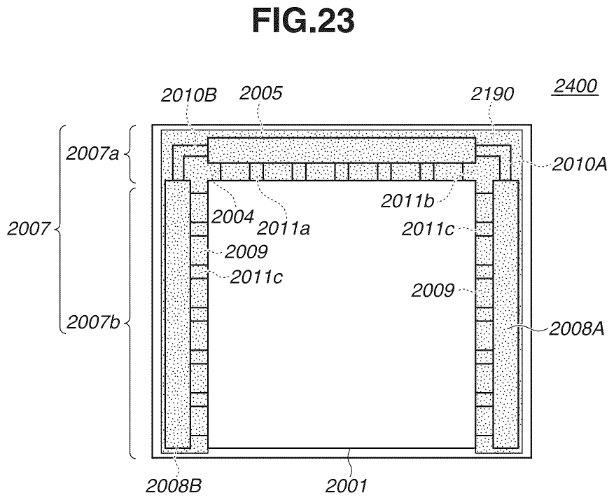

FIG. 23 is a schematic plan view illustrating a radiographic imaging apparatus including the current reduction mechanism according to the first aspect of the sixth exemplary embodiment.

In the first aspect, like the first aspect of the fourth exemplary embodiment, electromagnetic shields 2190 are disposed as the current reduction mechanism. The electromagnetic shields 2190 are disposed on the front and rear sides in the housing 2007 so that the radiation detection panel 2001, the signal detection circuits 2004, the control circuit 2005, the driving circuits 2008A and 2008B, and the connection lines 2010A and 2010B are covered, including the gaps 2011a, 2011b, and 2011c. With the electromagnetic shields 2190 provided in the radiographic imaging apparatus 2400, the gaps 2011a, 2011b, and 2011c are closed off with the electromagnetic shields 2190. This blocks the input of electromagnetic noise to the gaps 2011a, 2011b, and 2011c. The occurrence of loop currents in the closed circuits due to external electromagnetic noise is thereby prevented.

### —Second Aspect—

A second aspect of the current reduction mechanism according to the sixth exemplary embodiment will now be described.

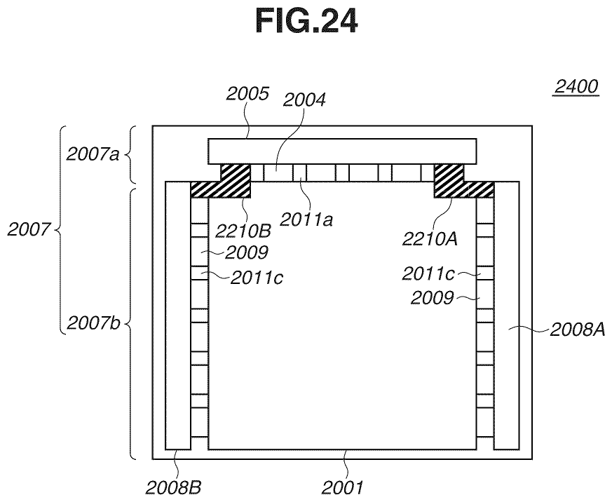

FIG. 24 is a schematic plan view illustrating a radiographic imaging apparatus including the current reduction mechanism according to the second aspect of the sixth exemplary embodiment.

In the second aspect, like the second aspect of the fourth exemplary embodiment, connection lines 2210A and 2210B are disposed as the current reduction mechanism instead of the connection lines 2010A and 2010B that form closed circuits. The connection line 2210A is connected to the control circuit 2005 at one end, passes over the rightmost signal detection circuit 2004 and a part of the radiation detection panel 2001, and is connected to the driving circuit 2008A at the other end. The control circuit 2005 and the driving circuit 2008A are thereby electrically connected. The connection line 2210B is connected to the control circuit 2005 at one end, passes over the leftmost signal detection circuit 2004 and a part of the radiation detection panel 2001, and is connected to the driving circuit 2008B at the other end. The control circuit 2005 and the driving circuit 2008B are thereby electrically connected.

In FIG. 22, the connection lines 2010A and 2010B constitute a part of closed circuits. Without the connection lines 2010A and 2010B, the GND loops are disconnected there, no closed circuit is formed, and no loop current occurs. In the second aspect, as illustrated in FIG. 24, the connection lines 2210A and 2210B are disposed instead of the connection lines 2010A and 2010B. The control circuit 2005 and the driving circuits 2008A and 2008B are thereby electrically connected without forming a closed circuit. In such a case, even if electromagnetic noise enters the gaps 2011a and 2011b, no loop current occurs since there is no closed circuit surrounding the gaps 2011a or 2011b.

Moreover, since the rightmost and leftmost signal detection circuits 2004 are covered by the connection lines 2210A and 2210B, the input of electromagnetic noise to the signal detection circuits 2004 is reduced by the connection lines 2210A and 2210B, whereby the occurrence of loop currents in the signal detection circuits 2004 is suppressed.

In the sixth exemplary embodiment, like the third aspect of the fourth exemplary embodiment, if the control circuit 2005 includes a stack of a plurality of circuit substrates, the signal detection circuits 2004 may be connected to the front surface of the first substrate 2021 and the connection lines that are the current reduction mechanism may be connected to the rear surface. This minimizes the amount of occurrence of loop currents in the control circuit 2005.

As described above, according to various aspects of the radiographic imaging apparatus of the sixth exemplary embodiment, the occurrence of loop currents due to external electromagnetic noise is reduced to suppress image noise and unexpected abnormal operation by simple techniques.

While the foregoing fourth to sixth exemplary embodiments have been described, each of the exemplary embodiments may be carried out by combining more than one of the first to third aspects. The foregoing fourth to sixth exemplary embodiments are merely examples of specific implementations for carrying out the present invention, and the technical scope of the present invention should not be interpreted as being limited thereto. In other words, the present invention may be practiced in various forms without departing from the technical concept or essential features thereof.

### Seventh Exemplary Embodiment

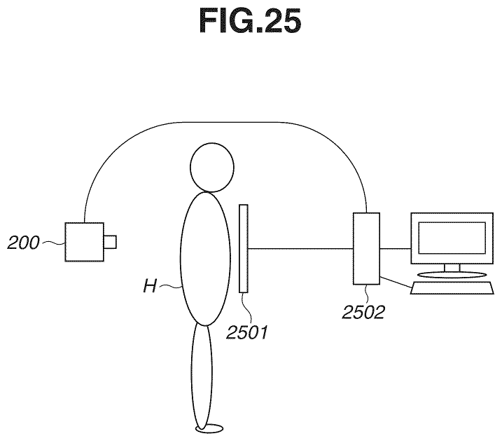

The radiographic imaging apparatuses according to the first to third aspects of the foregoing fourth to sixth exemplary embodiments may be applied to a radiographic imaging system illustrated in FIG. 25, for example.

This radiographic imaging system includes a radiographic imaging apparatus 2501 according to one of the first to third aspects of the foregoing fourth to sixth exemplary embodiments, a radiation generation apparatus 200, and a control and calculation processing apparatus 2502. The radiographic imaging apparatus 2501 and the radiation generation apparatus 200 are connected to the control and calculation processing apparatus 2502. The radiation generation apparatus 200 irradiates a subject H with radiation based on control of the control and calculation processing apparatus 2502. The radiographic imaging apparatus 2501 detects the radiation transmitted through the subject H. Information detected by the radiographic imaging apparatus 2051 is read into the control and calculation processing apparatus 2502 as electrical signals. The control and calculation processing apparatus 2502 performs desired calculation processing, and diagnosis is made.

According to the radiographic imaging system of the seventh exemplary embodiment, more accurate diagnosis can be made by using the radiographic imaging apparatus 2501 that reduces the occurrence of loop currents due to external electromagnetic noise and suppresses image noise and unexpected abnormal operation.

The fourth to seventh exemplary embodiments of the present invention include the following configurations.

### [Configuration 18]

A radiographic imaging apparatus comprising:
a radiation detection unit configured to detect radiation transmitted through a subject;
a signal detection circuit configured to detect a signal output from the radiation detection unit;
a signal processing circuit configured to process a signal output from the signal detection circuit;
a driving circuit configured to drive the radiation detection unit; and
a current reduction mechanism configured to reduce a loop current in a region where a closed circuit may occur.

### [Configuration 19]

The radiographic imaging apparatus according to Configuration 18, wherein the current reduction mechanism is disposed to cover at least an entry spot of electromagnetic noise in the region.

### [Configuration 20]

The radiographic imaging apparatus according to Configuration 19, wherein the current reduction mechanism is an electromagnetic shield that blocks input of electromagnetic noise.

### [Configuration 21]

The radiographic imaging apparatus according to Configuration 20, wherein the electromagnetic shield is disposed on at least one of an incident surface of the radiation and a rear surface of a side opposite to the incident surface.

### [Configuration 22]

The radiographic imaging apparatus according to Configuration 20, wherein the electromagnetic shield is disposed without overlapping the radiation detection unit in a plan view.

### [Configuration 23]

The radiographic imaging apparatus according to Configuration 18, wherein the current reduction mechanism is an electrical connection member which is a wiring route that does not form the closed circuit, among a plurality of wiring routes selectable in the region.

### [Configuration 24]

The radiographic imaging apparatus according to Configuration 23,
wherein the current reduction mechanism is an electrical connection member, and
wherein the electrical connection member is disposed to overlap the signal detection circuit at least in part and electrically connects the signal processing circuit and the driving circuit.

### [Configuration 25]

The radiographic imaging apparatus according to Configuration 24, wherein the electrical connection member is a flat flexible cable or flexible printed circuit.

### [Configuration 26]

The radiographic imaging apparatus according to Configuration 24, wherein the electrical connection member is a flat flexible cable or flexible printed circuit covered with a noise reduction member.

### [Configuration 27]

The radiographic imaging apparatus according to Configuration 24, wherein the electrical connection member overlaps a part of the radiation detection unit which is outside an effective pixel area in a plan view.

### [Configuration 28]

The radiographic imaging apparatus according to Configuration 18, wherein the current reduction mechanism is an electrical connection member which is a wiring route where the closed circuit has a smallest area, among a plurality of wiring routes selectable in the region.

### [Configuration 29]

The radiographic imaging apparatus according to Configuration 28, further including a control circuit,
wherein the control circuit includes at least
a first substrate including the signal processing circuit, and
a second substrate including another circuit, and
wherein the first substrate and the second substrate are electrically connected and stacked.

### [Configuration 30]

The radiographic imaging apparatus according to Configuration 29,
wherein the signal detection circuit is in contact with and electrically connected to one of a front surface and a rear surface of a circuit substate that is one of the first and second substrates, and
wherein the current reduction mechanism is an electrical connection member that is in contact with and electrically connected to the other of the front surface and the rear surface of the circuit substrate to which the signal detection circuit is connected.

### [Configuration 31]

The radiographic imaging apparatus according to Configuration 29,
wherein the signal detection circuit is in contact with one of a front surface and a rear surface of the first substrate and electrically connected to the signal processing circuit, and
wherein the current reduction mechanism is an electrical connection member that is in contact with the other of the front surface and the rear surface of the first substrate and electrically connected to the signal processing circuit.

### [Configuration 32]

The radiographic imaging apparatus according to any one of Configurations 18 to 31, wherein the driving circuit is disposed inside the radiation detection unit.

### [Configuration 33]

The radiographic imaging apparatus according to any one of Configurations 18 to 31, wherein at least two or more numbers of the driving circuits are disposed.

### [Configuration 34]

The radiographic imaging apparatus according to configuration 33, wherein two of the driving circuits are disposed at positions with the radiation detection unit held therebetween.

### [Configuration 35]

A radiographic imaging system including:
a radiation generation apparatus configured to irradiate a subject with radiation;
the radiographic imaging apparatus according to any one of Configurations 1 to 34; and
a calculation processing apparatus configured to perform predetermined calculation processing based on information obtained by the radiographic imaging apparatus.

According to the features set forth in Configurations 18 to 35, a radiographic imaging apparatus that reduces the occurrence of loop currents due to external electromagnetic noise and suppresses image noise and unexpected abnormal operation by simple techniques is implemented.

### Eighth Exemplary Embodiment

Next, an eighth exemplary embodiment will be described.

FIG. 26 is a diagram illustrating an example of a schematic configuration of a radiographic imaging system 10-8 according to the eighth exemplary embodiment. As illustrated in FIG. 26, the radiographic imaging system 10-8 includes a radiographic imaging apparatus 100, a radiation generation apparatus 200, a console 3300, a communication network 3400, an access point (AP) 3500, a connector 3600, and a cradle 3700. In the eighth exemplary embodiment, a case where the radiographic imaging system 10-8 operates in a synchronous imaging mode where the radiographic imaging apparatus 100 and the radiation generation apparatus 200 synchronously perform radiographic imaging of a subject H will be described.

The radiographic imaging apparatus 100 obtains a radiographic image of the subject H. The radiographic imaging apparatus 100 includes a wired or wireless communication function or both wired and wireless communication functions, and is configured to be able to transmit and receive information to/from the console 3300 via communication paths. In the example illustrated in FIG. 26, the radiographic imaging apparatus 100 is placed between a bed 30 and the subject H.

The radiation generation apparatus 200 includes a radiation tube 210 that emits radiation. In the example illustrated in FIG. 26, the radiation generation apparatus 200 is configured as a portable apparatus that is able to be brought into a hospital room and the like. In the example illustrated in FIG. 26, the radiation generation apparatus 200 is illustrated in a state of not performing radiographic imaging of the subject H. To perform radiographic imaging of the subject H, the radiation tube 210 of the radiation generation apparatus 200 is disposed so that the subject H is interposed between the radiation tube 210 and the radiographic imaging apparatus 100.

In the example illustrated in FIG. 26, the console 3300 is configured as a personal computer (PC) with a monitor or other display function and a user input function. This console 3300 transmits the user's input instructions to the radiographic imaging apparatus 100, receives radiographic image data obtained by the radiographic imaging apparatus 100, and displays the radiographic image data to the user. Moreover, the console 3300 includes a wired or wireless communication function or both wired and wireless communication functions. In the example illustrated in FIG. 26, the console 3300 is implemented as a laptop PC, whereas there are no particular restrictions in the actual operation of the radiographic imaging system 10-8. For example, the console 3300 may be installed as a stationary type, or built in the radiation generation apparatus 200.

An example of the communication network 3400 is a LAN network. For example, the radiographic imaging apparatus 100 and the console 3300 transmit and receive data to each other when connected to this communication network 3400.

The access point (AP) 3500 is connected for communication to the console 3300 via the communication network 3400, for example. The access point (AP) 3500 may be directly connected for communication to the console 3300, for example.

The connector 3600 connects the console 3300, the radiation generation apparatus 200, and the access point (AP) 3500 for communication, for example.

The cradle 3700 accommodates the radiographic imaging apparatus 100. Here, the cradle 3700 may include a power supply device inside to enable charging of the radiographic imaging apparatus 100.

In FIG. 26, the radiographic imaging apparatus 100 may transmit radiographic image data to the console 3300 via any of the communication network 3400 and the access point (AP) 3500 constituting the communication paths, depending on the configuration of the radiographic imaging system 10-8. The radiographic imaging apparatus 100 may directly transmit radiographic image data to the console 3300.

In FIG. 26, the solid lines and dotted lines indicate communication connections. Here, the dotted lines represent wireless connections. The radiographic imaging system 10-8 illustrated in FIG. 26 is configured so that the console 3300 and the radiographic imaging apparatus 100 are wirelessly connected, but may be configured so that the console 3300 and the radiographic imaging apparatus 100 are electrically connected using a cable or the like. If the radiographic imaging apparatus 100, the console 3300, and the access point (AP) 3500 have functions of directly transmitting and receiving data to/from each other, the radiographic imaging apparatus 100, the console 3300, and the access point (AP) 3500 may directly transmit and receive data to/from each other wirelessly or in a wired manner.

Next, an example of the procedure of radiographic imaging will be described. In the present exemplary embodiment, an operation in the synchronous imaging mode where the radiographic imaging apparatus 100 and the radiation generation apparatus 200 synchronously perform radiographic imaging will be described.

After the user such as a technician activates the radiographic imaging apparatus 100, the user operates the console 3300 to bring the radiographic imaging apparatus 100 into an imaging ready state. The user then operates the radiation generation apparatus 200 (including locating the radiation generation apparatus 200 so that the subject H is interposed between the radiation generation apparatus 200 and the radiographic imaging apparatus 100), and sets imaging conditions for radiation irradiation (such as the tube voltage and tube current of the radiation tube 210 and the irradiation time). After the end of the foregoing processing, the user checks whether the imaging preparations including the subject H are completed. The user then presses an exposure switch provided on the radiation generation apparatus 200 (or console 3300) to cause the radiation tube 210 of the radiation generation apparatus 200 emit (irradiate) radiation toward the subject H. When emitting the radiation, the radiation generation apparatus 200 transmits a signal indicating that radiation is about to be emitted to the radiographic imaging apparatus 100 via the connector 3600, the communication network 3400, or the like. The mode of transmission of the signal indicating that radiation will be emitted from the radiation generation apparatus 200 to the radiographic imaging apparatus 100 is not limited to via the connector 3600, the communication network 3400, or the like, and the signal may be directly transmitted.

When the radiographic imaging apparatus 100 receives the signal indicating that radiation will be emitted, the radiographic imaging apparatus 100 checks whether the preparations for radiation irradiation are completed, and if there are no issues, returns a radiation emission permission signal to the radiation generation apparatus 200. In response, the radiation generation apparatus 200 emits radiation.

In the present exemplary embodiment, the radiographic imaging apparatus 100 has an auto exposure control (AEC) function. In the present exemplary embodiment, the radiographic imaging apparatus 100 measures the radiation dose from the start of radiation irradiation, senses an appropriate radiation dose, and transmits the radiation dose to the console 3300. The console 3300 transmits an instruction to end radiation emission to the radiation generation apparatus 200 via the connector 3600.

The radiographic imaging apparatus 100 detects the end of radiation irradiation using various methods such as based on notification from the radiation generation apparatus 200 or referring to a set time determined in advance, and starts to generate radiographic image data. The generated radiographic image data is transmitted from the radiographic imaging apparatus 100 to the console 3300 through the communication paths illustrated in FIG. 26. The radiographic image data transmitted to the console 3300 is displayed on a display device included in the console 3300 as a radiographic image, for example.

Depending on conditions such as the imaging site of the subject H and the state of the subject H, radiographic imaging may be performed with the radiographic imaging apparatus 100 incorporated into an imaging gantry or the bed 30.

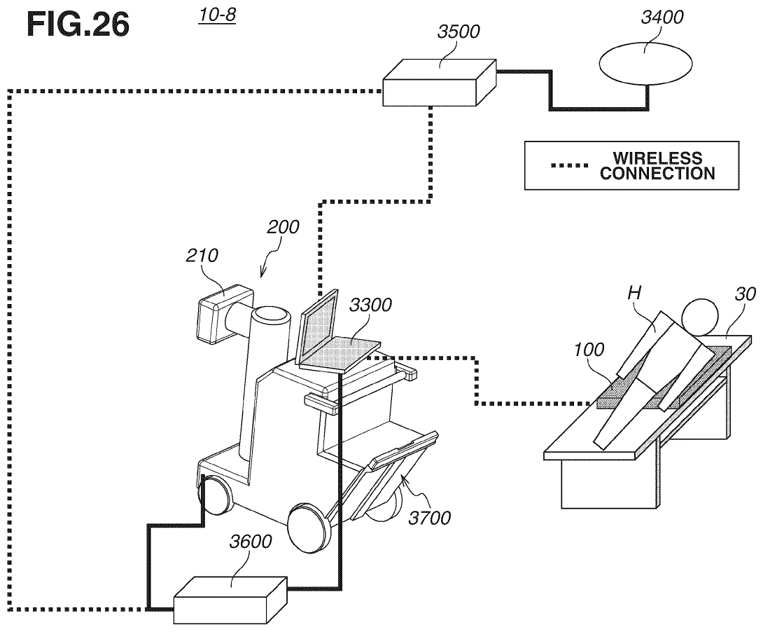

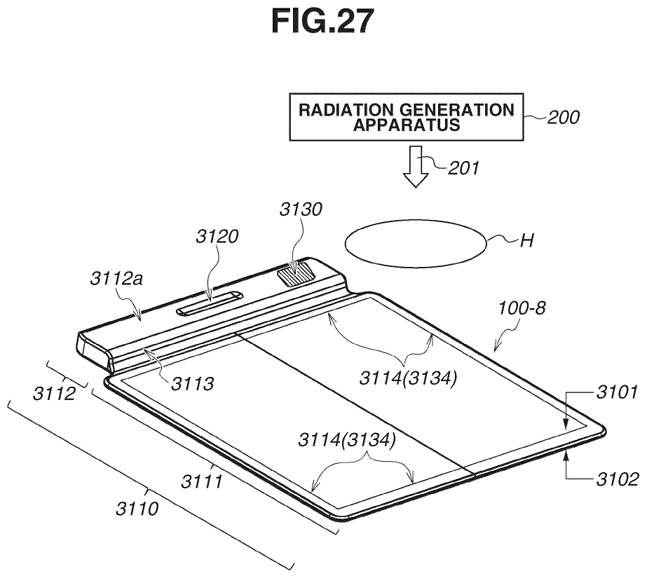

FIG. 27 is a diagram illustrating an example of the appearance of the radiographic imaging apparatus 100 according to the eighth exemplary embodiment. In this FIG. 27, components similar to those illustrated in FIG. 26 are denoted by the same reference numerals, and a detailed description thereof will be omitted. In the following description, the radiographic imaging apparatus 100 according to the eighth exemplary embodiment illustrated in FIG. 27 will be referred to as a “radiographic imaging apparatus 100-8”. In this FIG. 27, the radiation generation apparatus 200 (radiation tube 210) is disposed so that the subject H is interposed between the radiation generation apparatus 200 and the radiographic imaging apparatus 100-8. This FIG. 27 illustrates a state where the radiation generation apparatus 200 (radiation tube 210) emits radiation 201 toward the subject H and the radiographic imaging apparatus 100-8.

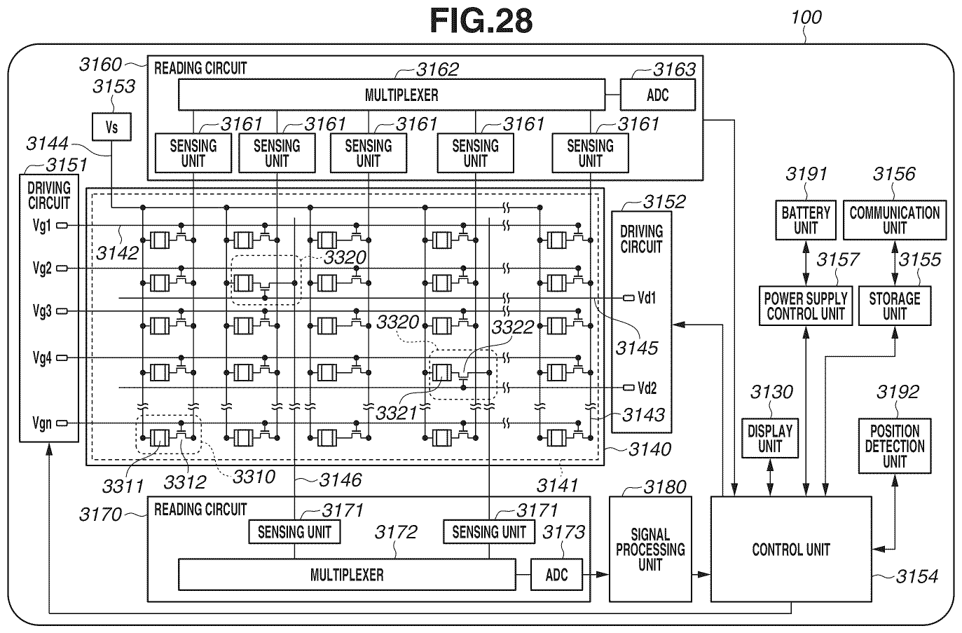

FIG. 27 illustrates a radiation incident surface 3101 where the radiation 201 is incident on the radiographic imaging apparatus 100-8, and a rear surface 3102 opposite to the radiation incident surface 3101. FIG. 27 also illustrates a housing 3110 of the radiographic imaging apparatus 100-8 as the appearance of the radiographic imaging apparatus 100-8. An indicator 3114 indicating the range of an effective imaging area 3141 where a radiation detection panel (radiation detection panel 3140 of FIG. 28 to be described below) accommodated in the housing 3110 detects the radiation 201 transmitted through the subject H is displayed on the housing 3110.

As illustrated in FIG. 27, the housing 3110 includes a thin section 3111 corresponding to a first thickness section that is a section including the effective imaging area 3141 as seen in the incident direction of the radiation 201 and has a first thickness in the incident direction of the radiation 201. As illustrated in FIG. 27, the housing 3110 also includes a thick section 3112 corresponding to a second thickness section that is a section not including the effective imaging area 3141 and has a second thickness greater than the thickness (first thickness) of the thin section 3111 in the incident direction of the radiation 201. More specifically, in the example illustrated in FIG. 27, the thick section (second thickness section) 3112 is thicker than the thin section (first thickness section) 3111 toward the side where the radiation 201 is incident. As illustrated in FIG. 27, the housing 3110 further includes a connection section 3113 that connects the thin section (first thickness section) 3111 and the thick section (second thickness section) 3112. The housing 3110 is configured as a single- or multi-part integral housing where the thin section (first thickness section) 3111, the thick section (second thickness section) 3112, and the connection section 3113 are integrated by the connection section 3113. Moreover, the thick section (second thickness section) 3112 of the housing 3110 is provided with a grip portion 3120 for the user to grip the housing 3110 and a display unit 3130 functioning as a user interface.

To achieve portability and strength of the radiographic imaging apparatus 100-8 in a compatible manner, the housing 3110 is suitably formed of materials such as magnesium alloys, aluminum alloys, fiber-reinforced plastic, and other plastics. However, other materials may be used. In particular, the radiation incident surface 3101 of the thin section 3111 where the effective imaging area 3141 is disposed is suitably formed of materials such as a carbon fiber-reinforced plastic with high transmittance for the radiation 201 and excellent lightweight properties. However, other materials may be used.

When radiographing the subject H such as a patient, the radiographic imaging apparatus 100-8 may be placed immediately behind the imaging site of the subject H. In doing so, due to a step created by the thickness of the radiographic imaging apparatus, the subject H and the end portion of the radiographic imaging apparatus come into contact to cause a reaction force, and the subject H (patient) may feel discomfort. In general, radiographic imaging apparatuses are often provided in sizes compliant with ISO (International Organization for Standardization) 4090:2001. In such a case, the radiographic imaging apparatuses are often configured with a thickness of approximately 15 mm to 16 mm. By contrast, in the present exemplary embodiment, the thin section 3111 of the housing 3110 has a thickness of 8.0 mm, which reduces the step created by the thickness of the radiographic imaging apparatus 100-8 during the radiographic imaging of the subject H. In the present exemplary embodiment, the reaction force caused by the contact between the subject H and the end portion of the radiographic imaging apparatus 100-8 is thus reduced, resulting in the effect of reducing burden and pain on the subject H. In the present exemplary embodiment, to obtain this effect, the thickness of the thin section 3111 of the housing 3110 is not limited to 8.0 mm and may be even smaller. The applicant has confirmed that the foregoing effect is obtainable if the thickness of the thin section 3111 of the housing 3110 is less than 10.0 mm. In the present exemplary embodiment, the thickness of the thin section 3111 of the housing 3110 is set to 8.0 mm as an appropriate thickness in view of various configurations and mechanical strength.

The grip portion 3120 is a portion where the user puts their hand in gripping the housing 3110. Specifically, the grip portion 3120 is disposed in a recessed shape in a first surface 3112a of the thick section 3112 of the housing 3110 whether the radiation 201 is incident. In the present exemplary embodiment, the grip portion 3120 is also disposed in a recessed shape in the surface of the thick section 3112 of the housing 3110 that is opposite to the first surface 3112a.

The display unit 3130 is a part functioning as a user interface. Specifically, in the example illustrated in FIG. 27, the display unit 3130 is disposed on the first surface 3112a of the thick section 3112 of the housing 3110 where the radiation 201 is incident. For example, the display unit 3130 sets regions of interest (ROIs) that are regions included in the effective imaging area 3141 and used for auto exposure control (AEC). Moreover, the display unit 3130 displays the state of the radiographic imaging apparatus 100-8, for example. This display unit 3130 is desirably a thin display with an input-capable touch sensor, for example, but may be a thin display with only display functionality and no touch sensor. To avoid interference with the grip portion 3120, this display unit 3130 is desirably disposed closer to an end rather than the center of the thick section 3112, for example.

As described above, the thin section 3111 of the housing 3110 according to the present exemplary embodiment contributes to a reduction in the burden and pain on the subject H (patient) during insertion into behind the subject H (patient). If, for example, the display unit is disposed on the thin section 3111 of the housing 3110, the user would have difficulty in visually observing the display unit because the thin section 3111 of the housing 3110 is hidden behind the subject H during the radiographic imaging of the subject H. By contrast, in the present exemplary embodiment, the display unit 3130 is disposed on the thick section 3112 of the housing 3110. The display unit 3130 is thus exposed outside the subject H even during the radiographic imaging of the subject H. This facilitates the user such as a technician to visually observe and operate the display unit 3130. Moreover, since the display unit 3130 is disposed on the thick section 3112 of the housing 3110, the display unit 3130 is disposed closer to the user during the radiographic imaging of the subject H, which is suitable in view of the user's visibility and operability. Thus, the radiographic imaging apparatus 100-8 according to the present exemplary embodiment reduces the burden and pain on the subject H (patient) and improve the user's visibility and operability of the display unit 3130 in a compatible manner.

FIG. 28 is a diagram illustrating an example of a functional configuration of the radiographic imaging apparatus 100 according to the eighth exemplary embodiment. As illustrated in FIG. 28, the radiographic imaging apparatus 100 includes functional components including the display unit 3130, a radiation detection panel 3140, driving circuits 3151 and 3152, an element power supply circuit 3153, a control unit 3154, a storage unit 3155, a communication unit 3156, and a power supply control unit 3157. As illustrated in FIG. 28, the radiographic imaging apparatus 100 further includes functional components including reading circuits 3160 and 3170, a signal processing unit 3180, a battery unit 3191, and a position detection unit 3192.

In the radiation detection panel 3140 illustrated in FIG. 28, the effective imaging area 3141 where the incident radiation 201 is detected is disposed within the thin section (first thickness section) 3111 of the housing 3110. A control substrate that controls driving of the radiation detection panel 3140 illustrated in FIG. 28 includes, for example, the driving circuits 3151 and 3152, the element power supply circuit 3153, the control unit 3154, the storage unit 3155, the communication unit 3156, and the power supply control unit 3157 illustrated in FIG. 28. This control substrate is accommodated in the thick section (second thickness section) 3112 of the housing 3110. A processing substrate that processes signals output from the radiation detection panel 3140 illustrated in FIG. 28 includes, for example, the reading circuits 3160 and 3170 and the signal processing unit 3180 illustrated in FIG. 28. This processing substrate is accommodated in the thick section (second thickness section) 3112 of the housing 3110. Note that the control substrate and the processing substrate described here do not need to be a single substrate, and may be composed of a plurality of substrates, for example. The battery unit 3191 that supplies power to the components of the radiographic imaging apparatus 100 is accommodated in the thick section (second thickness section) 3112 of the housing 3110. For example, a lithium-ion battery, an electric double layer capacitor, an all-solid-state battery, and the like are suitably used for the battery unit 3191, whereas other batteries may be used. The position detection unit 3192 that detects the position of the radiographic imaging apparatus 100 (for example, the installation position of the radiation detection panel 3140) is accommodated in the thick section (second thickness section) 3112 of the housing 3110.

The radiation detection panel 3140 has a function of detecting the incident radiation 201. The radiation detection panel 3140 includes a plurality of pixels arranged in a matrix to form a plurality of rows and a plurality of columns. The plurality of pixels described here includes a plurality of imaging pixels 3310 for obtaining radiographic image data and sensing pixels 3320 for sensing (monitoring) the amount of irradiation with the radiation 201. As illustrated in FIG. 28, an imaging pixel 3310 includes a first conversion element 3311 that converts the incident radiation 201 into an electrical signal, and a first switch element 3312 that is disposed between a column signal line 3143 and the first conversion element 3311. A sensing pixel 3320 includes a second conversion element 3321 that converts the incident radiation 201 into an electrical signal, and a second switch element 3322 that is disposed between a sensing signal line 3146 and the second conversion element 3321. The sensing pixel 3320 is arranged in the same column as some of the plurality of imaging pixels 3310. The sensing pixel 3320 may have a structure similar to that of the imaging pixel 3310.

In the radiation detection panel 3140, the first conversion elements 3311 and the second conversion elements 3321 include, for example, a scintillator that converts the radiation 201 into light and photoelectric conversion elements that convert the light generated by the scintillator into electrical signals. Here, the scintillator is typically formed in a sheet shape covering the effective imaging area 3141 and shared by the plurality of pixels. The first conversion elements 3311 and the second conversion elements 3321 may be composed of conversion elements that directly convert the radiation 201 into light, for example. The first switch elements 3312 and the second switch elements 3322 include, for example, thin-film transistors (TFTs) having an active region formed of a semiconductor such as amorphous silicon or polycrystalline silicon (desirably, polycrystalline silicon).

The radiation detection panel 3140 includes a plurality of drive lines 3142 and a plurality of column signal lines 3143. Each drive line 3142 corresponds to one of the plurality of rows in the effective imaging area 3141 and is driven by the driving circuit 3151. Each column signal line 3143 corresponds to one of the plurality of columns in the effective imaging area 3141. A first electrode of the first conversion element 3311 is connected to a first main electrode of the first switch element 3312. A second electrode of the first conversion element 3311 is connected to a bias line 3144. Here, each bias line 3144 extends in the column direction and is connected in common to the second electrodes of a plurality of first conversion elements 3311 arranged in the column direction. A bias voltage Vs is supplied from the element power supply circuit 3153 to the bias lines 3144. The control electrodes of the first switch elements 3312 in a plurality of imaging pixels 3310 constituting a single row are connected to one drive line 3142. The second main electrodes of the first switch elements 3312 in a plurality of imaging pixels 3310 constituting a single column are connected to one column signal line 3143.

The plurality of column signal lines 3143 is connected to the reading circuit 3160. Here, the reading circuit 3160 includes a plurality of sensing units 3161, a multiplexer 3162, and an analog-to-digital converter (hereinafter, referred to as an “AD converter”) 3163. The column signal lines 3143 are connected to respective corresponding ones of the plurality of sensing units 3161 of the reading circuit 3160. Here, one column signal line 3143 corresponds to one sensing unit 3161. The sensing units 3161 include differential amplifiers, for example. The multiplexer 3162 selects the plurality of sensing units 3161 in a predetermined order, and supplies the signal from the selected sensing unit 3161 to the AD converter 3163. The AD converter 3163 converts the supplied analog signal into a digital signal and outputs the digital signal as radiographic image data.

The radiographic image data digitized by the reading circuit 3160 is transmitted to the control unit 3154, and then transmitted to and stored in the storage unit 3155 by the control unit 3154. The radiographic image data stored in the storage unit 3155 may be immediately transmitted to an external apparatus (for example, the console 3300) via the communication unit 3156. The radiographic image data may be subjected to some processing by the control unit 3154 and then transmitted to an external apparatus (for example, the console 3300) via the communication unit 3156. The radiographic image data may be accumulated in the storage unit 3155.

The control unit 3154 performs processing related to the control of the components of the radiographic imaging apparatus 100. For example, the control unit 3154 outputs instructions for driving the radiation detection panel 3140 concerning radiographic imaging to the driving circuit 3151. The control unit 3154 may perform control to store the obtained radiographic image data into the storage unit 3155. The control unit 3154 may perform control to read the radiographic image data stored in the storage unit 3155 and transmit the radiographic image data to an external apparatus (for example, the console 3300) via the communication unit 3156. In addition to the transmission of the radiographic image data to an external apparatus via the communication unit 3156, the control unit 3154 receives instructions from the console 3300 and the like via the communication unit 3156. Moreover, the control unit 3154 performs switching operations such as activation/deactivation of the radiographic imaging apparatus 100 based on the user's operations from the display unit 3130. The control unit 3154 is also able to notify the user of the state of the radiographic imaging apparatus 100 (such as an operation status and an error state) via the display unit 3130. Furthermore, the control unit 3154 controls the driving circuits 3151 and 3152, the reading circuits 3160 and 3170, and the like based on information from the signal processing unit 3180, etc. In the present exemplary embodiment, the plurality of processes described above is performed by the single control unit 3154. However, for example, the radiographic imaging apparatus 100 may include a plurality of control units 3154 for respective predetermined functions, and the control units 3154 may perform processing in a distributed manner based on the respective functions. The control unit 3154 may be implemented using various components such as a CPU, MPU, FPGA, and CPLD, and there are no particular limitations on the specific components. Appropriate components may be selected and applied to the control unit 3154 depending on the functions and performance required of the radiographic imaging apparatus 100.

The storage unit 3155 is used to store the radiographic image data obtained by the radiographic imaging apparatus 100 and log information indicating the result of internal processing and the like. If the control unit 3154 is a CPU or the like, the storage unit 3155 stores programs to be executed by the CPU etc. Specific components of the storage unit 3155 are not limited in particular, and the storage unit 3155 may be implemented by various combinations of different types of memories and HDDs, whether volatile or nonvolatile. While FIG. 28 illustrates a single storage unit 3155, a plurality of storage units 3155 may be included in the radiographic imaging apparatus 100.

The communication unit 3156 performs processing for enabling communication between the radiographic imaging apparatus 100 and other apparatuses in the radiographic imaging system 10-8 excluding the radiographic imaging apparatus 100. The communication unit 3156 according to the present exemplary embodiment is capable of wireless communication and wired communication, and communicates with the console 3300, the access point (AP) 3500, and the like. The communication unit 3156 is not limited to the configuration described here, and may include only the wired communication function or the wireless communication function. The communication unit 3156 is not limited to any particular communication standard or method, either.

The power supply control unit 3157 controls the battery unit 3191 and the element power supply circuit 3153.

In the radiation detection panel 3140, a first electrode of the second conversion element 3321 is connected to a first main electrode of the second switch element 3322. A second electrode of the second conversion element 3321 is connected to a bias line 3144. The control electrode of the second switch element 3322 is electrically connected to a drive line 3145, and a second main electrode of the second switch element 3322 is connected to a sensing signal line 3146. Each drive line 3145 is connected with one or more sensing pixels 3320 and driven by the driving circuit 3152. Each sensing signal line 3146 is connected with one or more sensing pixels 3320. The plurality of sensing signal lines 3146 is connected to the reading circuit 3170. Here, the reading circuit 3170 includes a plurality of sensing units 3171, a multiplexer 3172, and an analog-to-digital converter (hereinafter, referred to as an “AD converter”) 3713. The sensing signal lines 3146 are connected to respective corresponding ones of the plurality of sensing units 3171 of the reading circuit 3170. Here, one sensing signal line 3146 corresponds to one sensing unit 3171. The sensing units 3171 include differential amplifiers, for example. The multiplexer 3172 selects the plurality of sensing units 3171 in a predetermined order, and supplies the signal from the selected sensing unit 3171 to the AD converter 3173. The AD converter 3173 converts the supplied analog signal into a digital signal and outputs the digital signal.

The output signal from the reading circuit 3170 (specifically, AD converter 3173) is supplied to the signal processing unit 3180 and processed by the signal processing unit 3180. Based on the output signal from the reading circuit 3170 (AD converter 3173), the signal processing unit 3180 outputs information about the irradiation of the radiographic imaging apparatus 100 with the radiation 201. Specifically, as the information about the irradiation with the radiation 201, the signal processing unit 3180 outputs, for example, information that the irradiation of the radiographic imaging apparatus 100 with the radiation 201 is detected, and information about the dose (cumulative dose) of the radiation 201 emitted under AEC. Based on the information output from the signal processing unit 3180, if an appropriate dose (cumulative dose) of the radiation 201 is reached, the control unit 3154 then controls the amount of irradiation of the subject H with the radiation 201 by notifying the radiation generation apparatus 200 to stop emitting the radiation 201. To appropriately detect the dose (cumulative dose) of exposure to the radiation 201, the radiographic imaging apparatus 100 needs to use sensing pixels 3320 at the location where the subject H is positioned. In such a case, the control unit 3154 selects the sensing pixels 3320 to be driven based on selection information about ROIs to be used for AEC from the display unit 3130, for example.

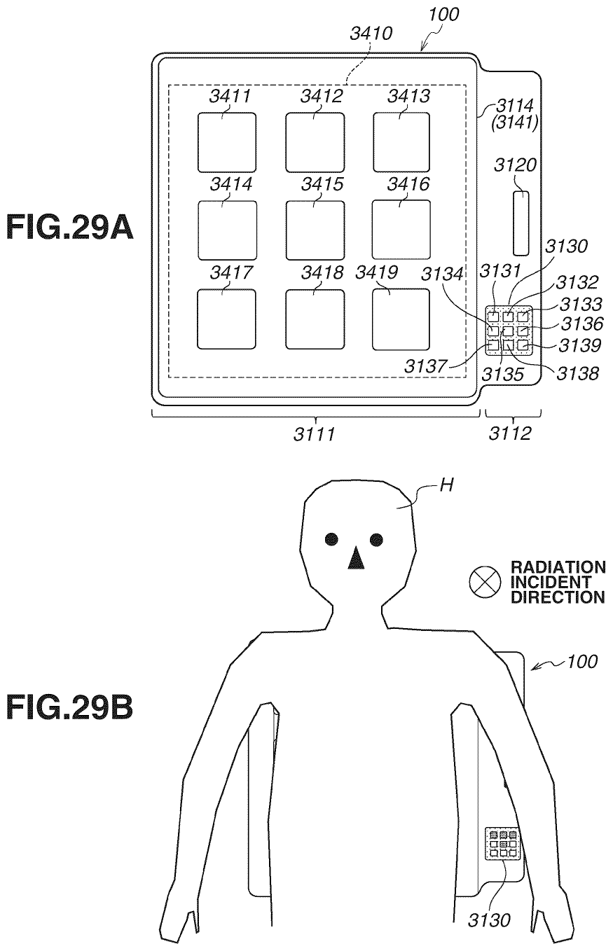

FIGS. 29A and 29B are diagrams for describing a selection example of ROIs to be used for AEC, using the display unit 3130 on the radiographic imaging apparatus 100 according to the eighth exemplary embodiment. In these FIGS. 29A and 29B, components similar to those illustrated in FIGS. 26 to 28 are denoted by the same reference numerals, and a detailed description thereof will be omitted.

FIG. 29A is an external view of the radiographic imaging apparatus 100 seen from the side where the radiation 201 is incident. In the radiographic imaging apparatus 100 illustrated in FIG. 29A, a region of interest (ROI) 3410 needed for auto exposure control (AEC) is set within the effective imaging area 3141 disposed in the thin section 3111 of the housing 3110. The ROI 3410 includes nine regions of interest, or ROIs, 3411 to 3419. While in the example illustrated in FIG. 29A the nine ROIs 3411 to 3419 are set in the ROI 3410, the present exemplary embodiment is not limited thereto. For example, 12 ROIs may be set.

The display unit 3130 displays a rectangle having a shape similar to that of the ROI 3410 based on the orientation of the effective imaging area 3141. The display unit 3130 also displays display areas 3131 to 3139 corresponding to the nine ROIs 3411 to 3419 included in the ROI 3410, respectively. When radiographing the subject H, the user can set the regions of interest to be used for AEC by directly touching and selecting the display areas 3131 to 3139 corresponding to the ROIs 3411 to 3419 to be selected, using the display unit 3130.

For example, a case of radiographing the chest (lung fields) of the subject H will be described.

If, for example, the user wants to set the ROIs 3411, 3412, 3413, and 3415 as the regions of interest to be used for AEC, the user selects the corresponding display areas 3131, 3132, 3133, and 3135 on the display unit 3130.

When the user selects display areas on the display unit 3130, the color of the selected display areas changes to clearly indicate the selected locations as illustrated in the display unit 3130 of FIG. 29B, for example.

FIG. 29B illustrates an example where the thick section (second thickness section) 3112 of the housing 3110 is on the left of the subject H facing the incident direction of the radiation 201. If the radiographic imaging apparatus 100 is 180° rotated relative to the state illustrated in FIG. 29B and the thick section (second thickness section) 3112 of the housing 3110 is on the right of the subject H, the display areas of the display unit 3130 corresponding to the ROIs 3415 and 3417 to 3419 are to be selected.

The radiographic imaging apparatus 100 according to the eighth exemplary embodiment includes the display unit 3130 functioning as a user interface on the thick section 3112 of the housing 3110, which is thicker in the incident direction of the radiation 201 than the thin section 3111 where the effective imaging area 3141 is disposed

Such a configuration facilitates information exchange between the radiographic imaging apparatus 100 and the user. As a comparative example, in which the display unit 3130 is disposed on the thin section 3111 of the housing 3110 where the effective imaging area 3141 is disposed, the user has difficulty in visually observing the display unit 3130 when radiographing the subject H, since the thin section 3111 of the housing 3110 is hidden behind the subject H. In this comparative example, if the display unit 3130 has the operation function, malfunction may result from contact with the arms or legs of the subject H. By contrast, in the present exemplary embodiment, the display unit 3130 is disposed on the thick section 3112 of the housing 3110. The display unit 3130 is thus exposed outside the subject H even during the radiographic imaging of the subject H, and the user can visually observe and operate the display unit 3130 easily. Moreover, since the display unit 3130 is disposed on the thick section 3112 of the housing 3110, the display unit 3130 is disposed close to the user when radiographing the subject H, which is suitable in terms of the user's visibility and operability.

### Ninth Exemplary Embodiment

Next, a ninth exemplary embodiment will be described. In the following description of the ninth exemplary embodiment, a description of items common to the foregoing eighth exemplary embodiment is omitted, and differences from the foregoing eighth exemplary embodiment will be described.

A radiographic imaging system according to the ninth exemplary embodiment has a schematic configuration similar to that of the radiographic imaging system 10 according to the eighth exemplary embodiment illustrated in FIG. 26. The appearance of a radiographic imaging apparatus 100 according to the ninth exemplary embodiment is also similar to that of the radiographic imaging apparatus 100 according to the eighth exemplary embodiment illustrated in FIG. 27. The radiographic imaging apparatus 100 according to the ninth exemplary embodiment also has a functional configuration similar to that of the radiographic imaging apparatus 100 according to the eighth exemplary embodiment illustrated in FIG. 28.

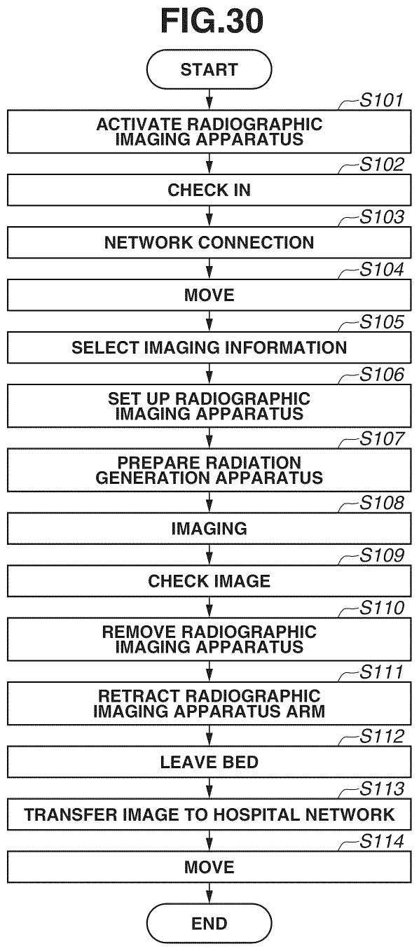

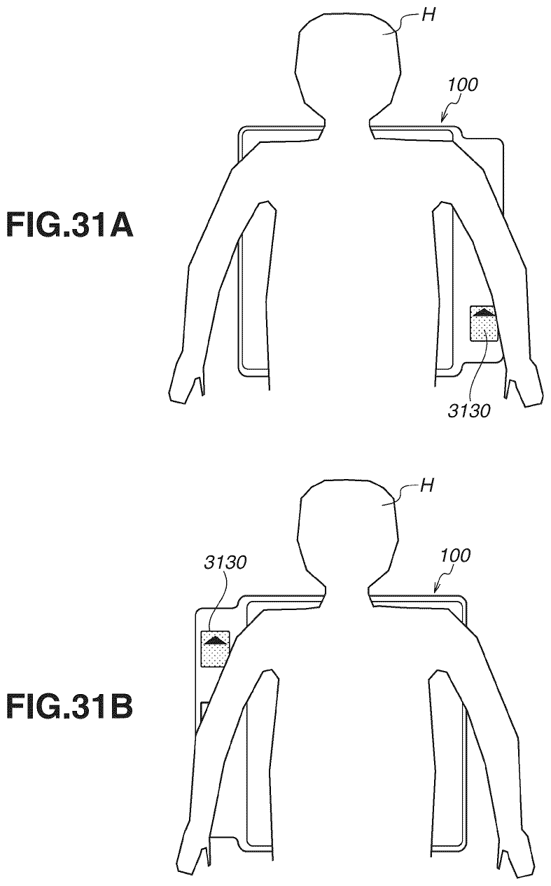

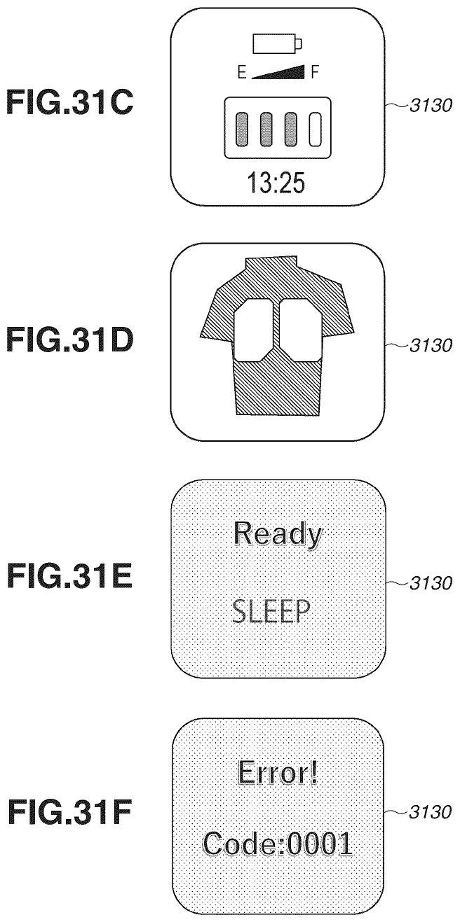

FIG. 30 is a flowchart illustrating an example of a processing procedure for a radiographic imaging method of the radiographic imaging system 10 according to the ninth exemplary embodiment. FIGS. 31A to 31F are diagrams illustrating display examples of the display unit 3130 of the radiographic imaging apparatus 100 according to the ninth exemplary embodiment. In FIGS. 31A to 31F, components similar to those illustrated in FIGS. 26 to FIGS. 29A and 29B are denoted by the same reference numerals, and a detailed description thereof will be omitted. The flowchart illustrated in FIG. 30 will now be described with reference to FIGS. 31A to 31F as needed.

Initially, in step S101 of FIG. 30, the user such as a technician activates the radiographic imaging apparatus 100. With the radiographic imaging apparatus 100 activated, the display unit 3130 displays information indicating the state of the radiographic imaging apparatus 100, such as remaining level information about the battery unit 3191 and time information as illustrated in FIG. 31C.

Next, in step S102 of FIG. 30, the patient who is the subject H checks in at the hospital or the like. Then, in step S103 of FIG. 30, the radiographic imaging system 10 establishes a network connection.

Next, in step S104 of FIG. 30, the patient who is the subject H moves to the hospital room or the like. Then, in step S105 of FIG. 30, the user selects imaging information about the subject H. Here, the user operates the console 3300 to select an imaging protocol, for example. The display unit 3130 displays the selected imaging protocol as illustrated in FIG. 31D, for example.

Next, in step S106 of FIG. 30, the user sets up the radiographic imaging apparatus 100 for the patient who is the subject H.

Next, in step S107 of FIG. 30, the user sets the imaging conditions for the irradiation with radiation 201 (such as the tube voltage and tube current of the radiation tube 210 of the radiation generation apparatus 200, and irradiation time) to prepare the radiation generation apparatus 200.

In steps S106 and S107 of FIG. 30, if the radiation generation apparatus 200 and the radiographic imaging apparatus 100 are synchronized, the display unit 3130 may display the conditions of the radiation generation apparatus 200. Alternatively, when the radiographic imaging apparatus 100 is set under the subject H etc., the radiographic imaging apparatus 100 recognizes and detects that the subject H is in contact, using touch sensors (not illustrated) disposed on the outer periphery of the thin section 3111. If the contact between the radiographic imaging apparatus 100 and the subject H is recognized, the display unit 3130 may automatically switch to the display of the imaging protocol and the operation state of the radiographic imaging apparatus 100. To prevent the display of the display unit 3130 from being unintentionally switched by the subject H or the like, the input from the display unit 3130 may be locked when the contact between the radiographic imaging apparatus 100 and the subject H is recognized.

In steps S106 and S107 of FIG. 30, if the radiation generation apparatus 200 and the radiographic imaging apparatus 100 are not synchronized, the signal threshold for detecting the radiation 201 may be changed depending on the presence or absence of the subject H so that erroneous detection is less likely to occur. For example, when the contact between the radiographic imaging apparatus 100 and the subject H is not recognized, the signal threshold for detecting the radiation 201 is increased to prevent erroneous detection due to noise, vibration, and the like from nearby devices, and the display unit 3130 displays that the threshold is increased. Subsequently, when the contact between the radiographic imaging apparatus 100 and the subject H is recognized, the original threshold may be restored and the display unit 3130 may be controlled to display the state capable of detecting the radiation 201.

FIGS. 31A and 31B illustrate cases where the chest (lung fields) of the subject H is radiographed. Here, as illustrated in FIGS. 31A and 31B, a triangle (in the example illustrated in FIGS. 31A and 31B, a triangle indicating the top of the radiographic imaging apparatus 100) or other indications may be displayed on the display unit 3130 so that the up-down orientation of the radiographic imaging apparatus 100 can be seen.

Here, in the present exemplary embodiment, the display unit 3130 displays the triangle or other indications indicating the up-down orientation of the radiographic imaging apparatus 100 based on position information about the radiographic imaging apparatus 100 detected by the position detection unit 3192, which includes a gyro sensor, an angle sensor, or the like, for example.

Next, in step S108 of FIG. 30, the radiographic imaging apparatus 100 performs radiographic imaging of the subject H. Since the radiographic imaging apparatus 100 takes several seconds of waiting time to become ready for radiographic imaging, the display unit 3130 displays information indicating the preparation state as illustrated in FIG. 31E, for example. To transition to the state ready for radiographic imaging, the display unit 3130 may be operated instead of the console 3300. If, for example, the radiation generation apparatus 200 and the radiographic imaging apparatus 100 are not synchronized and the radiation 201 is detected, the display unit 3130 displays the detection of the radiation 201.

If the radiographic imaging apparatus 100 is in an abnormal state in any of the steps in FIG. 30, the display unit 3130 displays information that the radiographic imaging apparatus 100 is in an abnormal state as illustrated in FIG. 31F. The user can operate the console 3300 or contact the serviceperson based on the error code displayed on the display unit 3130.

Next, in step S109 of FIG. 30, the user checks the radiographic image obtained as a result of the radiographic imaging of the subject H in step S108. For example, the user checks the radiographic image displayed on the console 3300.

Next, if there are no issues as a result of the checking of the radiographic image in step S109, then in step S110 of FIG. 30, the user removes the radiographic imaging apparatus 100 used for the radiographic imaging of the subject H. Next, in step S111 of FIG. 30, the user stores the radiographic imaging apparatus 100 removed in step S110 into the cradle 3700.

Next, in step S112 of FIG. 30, the patient who is the subject H leaves the bed 30 where the subject H has been lying for the radiographic imaging. Next, in step S113 of FIG. 30, the radiographic imaging apparatus 100 and the console 3300 transmit (transfer) the radiographic image obtained as a result of the radiographic imaging of the subject H in step S108 to the hospital network. Next, in step S114 of FIG. 30, the patient who is the subject H and the user such as a technician move out of the hospital room or the like. When step S114 is completed, the processing of the flowchart illustrated in FIG. 30 ends.

The ninth exemplary embodiment, like the eighth exemplary embodiment, also facilitates information exchange between the radiographic imaging apparatus 100 and the user.

### Tenth Exemplary Embodiment

Next, a tenth exemplary embodiment will be described. In the following description of the tenth exemplary embodiment, a description of items common to the foregoing eighth and ninth exemplary embodiments is omitted, and differences from the foregoing eighth and ninth exemplary embodiments will be described.

A radiographic imaging system according to the tenth exemplary embodiment has a schematic configuration similar to that of the radiographic imaging system 10 according to the eighth exemplary embodiment illustrated in FIG. 26. Moreover, a radiographic imaging apparatus 100 according to the tenth exemplary embodiment has a functional configuration similar to that of the radiographic imaging apparatus 100 according to the eighth exemplary embodiment illustrated in FIG. 28.

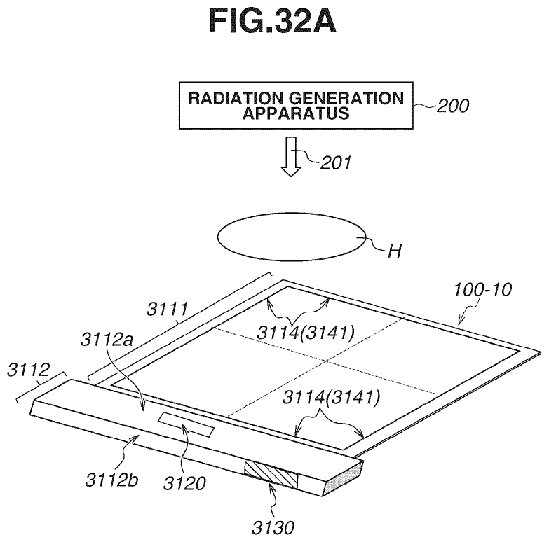

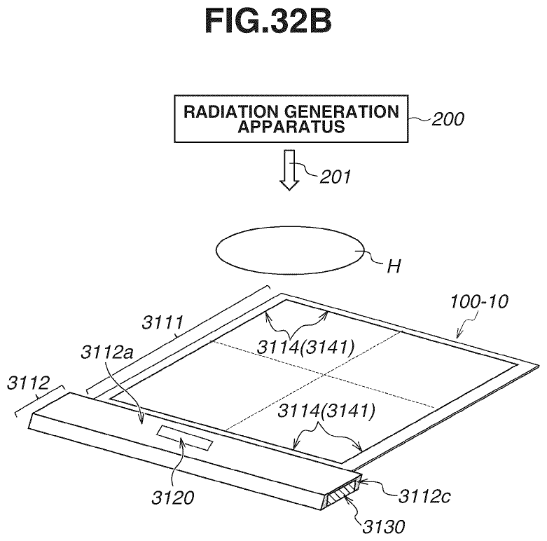

FIGS. 32A and 32B are diagrams illustrating an example of the appearance of the radiographic imaging apparatus 100 according to the tenth exemplary embodiment. In these FIGS. 32A and 32B, components similar to those illustrated in FIGS. 26 and 27 are denoted by the same reference numerals, and a detailed description thereof will be described. In the following description, the radiographic imaging apparatus 100 according to the tenth exemplary embodiment illustrated in FIGS. 32A and 32B will be referred to as a “radiographic imaging apparatus 100-10”. In these FIGS. 32A and 32B, the radiation generation apparatus 200 (radiation tube 210) is disposed so that the subject H is interposed between the radiation generation apparatus 200 and the radiographic imaging apparatus 100-10. These FIGS. 32A and 32B illustrate a state where the radiation generation apparatus 200 (radiation tube 210) emits radiation 201 toward the subject H and the radiographic imaging apparatus 100-10.

In the radiographic imaging apparatus 100-10 illustrated in FIG. 32A, the display unit 3130 is disposed on a second surface 3112b of the thick section 3112 of the housing 3110 different from the first surface 3112a where the radiation 201 is incident. In this FIG. 32A, the second surface 3112b corresponds to a side surface on a long-edge side of the thick section 3112 of the housing 3110.

In the radiographic imaging apparatus 100-10 illustrated in FIG. 32B, the display unit 3130 is disposed on a second surface 3112c of the thick section 3112 of the housing 3110 different from the first surface 3112a where the radiation 201 is incident. In this FIG. 32B, the second surface 3112c corresponds to a side surface on a short-edge side of the thick section 3112 of the housing 3110.

Depending on the subject H and the layout of the bed 30 and the like in the hospital room, the user may have difficulty in visually observing or operating the display unit 3130 if the display unit 3130 is disposed on the first surface 3112a where the radiation 201 is incident. In such a case, a display unit 3130 easier to visually observe and operate may be provided by locating the display unit 3130 on a side surface of the thick section 3112 of the housing 3110 as illustrated in FIGS. 32A and 32B.

The tenth exemplary embodiment, like the eighth exemplary embodiment, also facilitates information exchange between the radiographic imaging apparatus 100 and the user.

### Eleventh Exemplary Embodiment

Next, an eleventh exemplary embodiment will be described. In the following description of the eleventh exemplary embodiment, a description of items common to the foregoing eighth to tenth exemplary embodiments is omitted, and differences from the foregoing eighth to tenth exemplary embodiments will be described.

A radiographic imaging system according to the eleventh exemplary embodiment has a schematic configuration similar to that of the radiographic imaging system 10 according to the eighth exemplary embodiment illustrated in FIG. 26. A radiographic imaging apparatus 100 according to the eleventh exemplary embodiments has a functional configuration similar to that of the radiographic imaging apparatus 100 according to the eighth exemplary embodiment illustrated in FIG. 28.

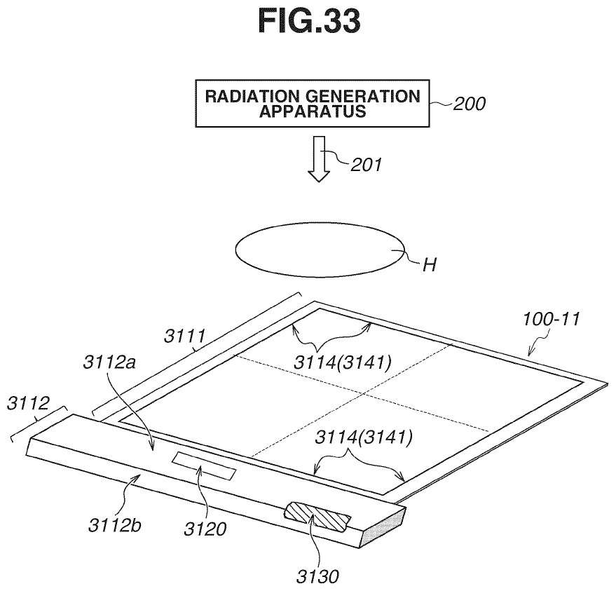

FIG. 33 is a diagram illustrating an example of the appearance of the radiographic imaging apparatus 100 according to the eleventh exemplary embodiment. In this FIG. 33, components similar to those illustrated in FIGS. 26, 27, 32A, and 32B are denoted by the same reference numerals, and a detailed description thereof will be omitted. In the following description, the radiographic imaging apparatus 100 according to the eleventh exemplary embodiment illustrated in FIG. 33 will be referred to as a “radiographic imaging apparatus 100-11”. In this FIG. 33, the radiation generation apparatus 200 (radiation tube 210) is disposed so that the subject H is interposed between the radiation generation apparatus 200 and the radiographic imaging apparatus 100-11. This FIG. 33 illustrates a state where the radiation generation apparatus 200 (radiation tube 210) emits radiation 201 toward the subject H and the radiographic imaging apparatus 100-11.

In the radiographic imaging apparatus 100-11 illustrated in FIG. 33, the display unit 3130 is disposed across the first surface 3112a of the thick section 3112 of the housing 3110 where the radiation 201 is incident and the second surface 3112b different from the first surface 3112a. In this FIG. 33, the second surface 3112b corresponds to a side surface on a long-edge side of the thick section 3112 of the housing 3110.

The display unit 3130 illustrated in FIG. 33 may be composed of a flexible-type display. The first surface 3112a and the second surface 3112b of the thick section 3112 may be chamfered or otherwise machined flat, and a flat display may be disposed thereon. The layout of the display unit 3130 illustrated in this FIG. 33 is effective in cases where the display unit 3130 is difficult to visually observe or operate from only the first surface 3112a or only the second surface 3112b of the thick section 3112.

The eleventh exemplary embodiment, like the eighth exemplary embodiment, also facilitates information exchange between the radiographic imaging apparatus 100 and the user.

### Twelfth Exemplary Embodiment

Next, a twelfth exemplary embodiment will be described. In the following description of the twelfth exemplary embodiment, a description of items common to the foregoing eighth to eleventh exemplary embodiments is omitted, and differences from the foregoing eighth to eleventh exemplary embodiments will be described.

A radiographic imaging system according to the twelfth exemplary embodiment has a schematic configuration similar to that of the radiographic imaging system 10 according to the eighth exemplary embodiment illustrated in FIG. 26. A radiographic imaging apparatus 100 according to the twelfth exemplary embodiment has a functional configuration similar to that of the radiographic imaging apparatus 100 according to the eighth exemplary embodiment illustrated in FIG. 28.

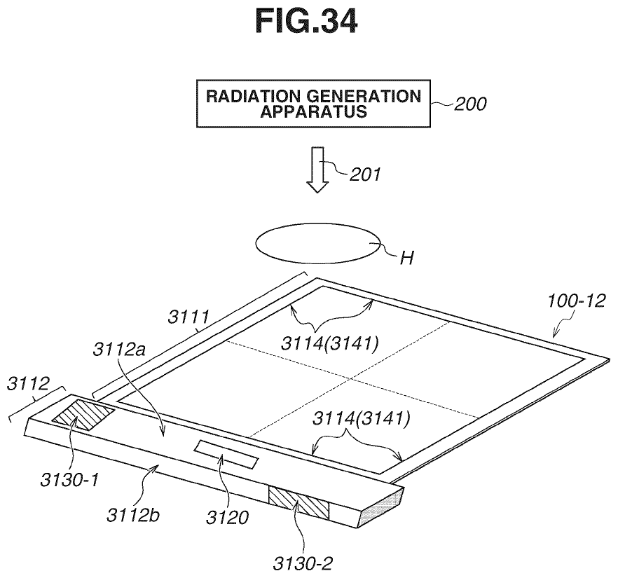

FIG. 34 is a diagram illustrating an example of the appearance of the radiographic imaging apparatus 100 according to the twelfth exemplary embodiment. In this FIG. 34, components similar to those illustrated in FIGS. 26, 27, 32A, 32B, and 33 are denoted by the same reference numerals, and a detailed description thereof will be omitted. In the following description, the radiographic imaging apparatus 100 according to the twelfth exemplary embodiment illustrated in FIG. 34 will be referred to as a “radiographic imaging apparatus 100-12”. In this FIG. 34, the radiation generation apparatus 200 (radiation tube 210) is disposed so that the subject H is interposed between the radiation generation apparatus 200 and the radiographic imaging apparatus 100-12. This FIG. 34 illustrates a state where the radiation generation apparatus 200 (radiation tube 210) emits radiation 201 toward the subject H and the radiographic imaging apparatus 100-12.

In the radiographic imaging apparatus 100-12 illustrated in FIG. 34, a plurality of display units 3130-1 and 3130-2 is disposed as display units 3130 at a plurality of positions on the thick section 3112 of the housing 3110. Specifically, in the radiographic imaging apparatus 100-12 illustrated in FIG. 34, a first display unit 3130-1 is disposed on the first surface 3112a of the thick section 3112 of the housing 3110 where the radiation 201 is incident, and a second display unit 3130-2 is disposed on the second surface 3112b different from the first surface 3112a. In this FIG. 34, the second surface 3112b corresponds to a side surface on a long-edge side of the thick section 3112 of the housing 3110.

In the radiographic imaging apparatus 100-12 illustrated in FIG. 34, for example, the first display unit 3130-1 functions as a main display unit, and the second display unit 3130-2 functions as a sub display unit. Here, the functions may be divided such that the first display unit 3130-1 is used to set ROIs to be used for AEC as in the eighth exemplary embodiment, for example, and the second display unit 3130-2 displays the remaining level information about the battery unit 3191, time information, and the like as illustrated in FIG. 31C, for example.

As illustrated in this FIG. 34, the plurality of display units 3130-1 and 3130-2 is disposed on the thick section 3112 of the housing 3110. Even if the visibility or operability of one display unit 3130 is impaired due to factors such as the layout of the subject H and the bed 30, the other display unit 3130 ensures visibility and operability.

In the example illustrated in FIG. 34, the display units 3130-1 and 3130-2 are disposed on different surfaces of the thick section 3112 of the housing 3110. However, a configuration where the display units 3130-1 and 3130-2 are disposed on the same surface of the thick section 3112 of the housing 3110, for example, is also included in the present exemplary embodiment.

The twelfth exemplary embodiment, like the eighth exemplary embodiment, also facilitates information exchange between the radiographic imaging apparatus 100 and the user.

Note that all the foregoing eighth to twelfth exemplary embodiments of the present invention are merely examples of specific implementations for carrying out the present invention, and the technical scope of the present invention should not be interpreted as being limited thereto. In other words, the present invention may be practiced in various forms without departing from the technical concept or essential features thereof.

The eighth to twelfth exemplary embodiments of the present invention include the following configurations.

### [Configuration 36]

A radiographic imaging apparatus comprising:
a radiation detection panel configured to include an effective imaging area where incident radiation is detected;
a housing configured to accommodate the radiation detection panel; and
a display unit configured to function as a user interface,
wherein the housing includes
a first thickness section, having a first thickness in an incident direction of the radiation, where the effective imaging area is disposed, and
a second thickness section, having a second thickness greater than the first thickness in the incident direction of the radiation, where the display unit is disposed.

### [Configuration 37]

The radiographic imaging apparatus according to Configuration 36,
wherein the radiographic imaging apparatus has a function of auto exposure control, and
wherein the display unit is capable of setting a region to be used for the auto exposure control, the region being included in the effective imaging area.

### [Configuration 38]

The radiographic imaging apparatus according to Configuration 36, wherein the display unit displays a state of the radiographic imaging apparatus.

### [Configuration 39]

The radiographic imaging apparatus according to any one of Configurations 36 to 38, wherein the display unit is disposed on a first surface of the second thickness section where the radiation is incident.

### [Configuration 40]

The radiographic imaging apparatus according to any one of Configurations 36 to 38, wherein the display unit is disposed on a second surface of the second thickness section, the second surface being different from the first surface where the radiation is incident.

### [Configuration 41]

The radiographic imaging apparatus according to any one of Configurations 36 to 38, wherein the display unit is disposed across the first surface of the second thickness section where the radiation is incident and the second surface different from the first surface.

### [Configuration 42]

The radiographic imaging apparatus according to any one of Configurations 36 to 41, wherein the display unit is disposed at a plurality of positions on the second thickness section.

### [Configuration 43]

The radiographic imaging apparatus according to any one of Configurations 36 to 42, further including a control substrate configured to control driving of the radiation detection panel,
wherein the second thickness section accommodates the control substrate.

### [Configuration 44]

The radiographic imaging apparatus according to any one of Configurations 36 to 43, further including a processing substrate configured to process a signal output from the radiation detection panel,
wherein the second thickness section accommodates the processing substrate.

### [Configuration 45]

The radiographic imaging apparatus according to any one of Configurations 36 to 44, further including a battery unit configured to supply power to the radiographic imaging apparatus,
wherein the second thickness section accommodates the battery unit.

### [Configuration 46]

The radiographic imaging apparatus according to any one of Configurations 36 to 45,
wherein the housing further includes a connection section connecting the first thickness section and the second thickness section, and
wherein the first thickness section, the second thickness section, and the connection section of the housing are integrated by the connection section.

### [Configuration 47]

The radiographic imaging apparatus according to any one of Configurations 36 to 46, further including a grip portion configured to be gripped to hold the housing,
wherein the grip portion is formed in a recessed shape in the second thickness section.

### [Configuration 48]

The radiographic imaging apparatus according to any one of Configurations 36 to 47, wherein the second thickness section is thicker than the first thickness section toward a side where the radiation is incident.

### [Configuration 49]

A radiographic imaging system comprising:
the radiographic imaging apparatus according to any one of Configurations 36 to 48; and
a radiation generation apparatus configured to generate the radiation.

According to the features set forth in Configurations 36 to 49, information exchange between the radiographic imaging apparatus and the user is facilitated.

### Thirteenth Exemplary Embodiment

Next, a thirteenth exemplary embodiment will be described.

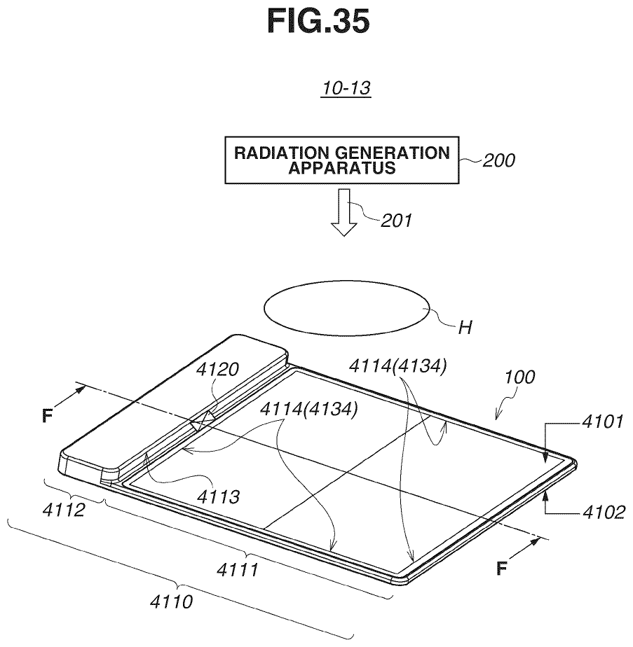

FIG. 35 is a diagram illustrating an example of a schematic configuration of a radiographic imaging system 10-13 according to the thirteenth exemplary embodiment. The radiographic imaging system 10-13 includes a radiographic imaging apparatus 100 and a radiation generation apparatus 200.

The radiation generation apparatus 200 is an apparatus that emits radiation 201 toward a subject H and the radiographic imaging apparatus 100.

The radiographic imaging apparatus 100 is an apparatus that detects the incident radiation 201 (including the radiation 201 transmitted through the subject H) and obtains a radiographic image of the subject H. FIG. 35 illustrates a radiation incident surface 4101 of the radiographic imaging apparatus 100 where the radiation is incident and a rear surface 4102 opposite to the radiation incident surface 4101.

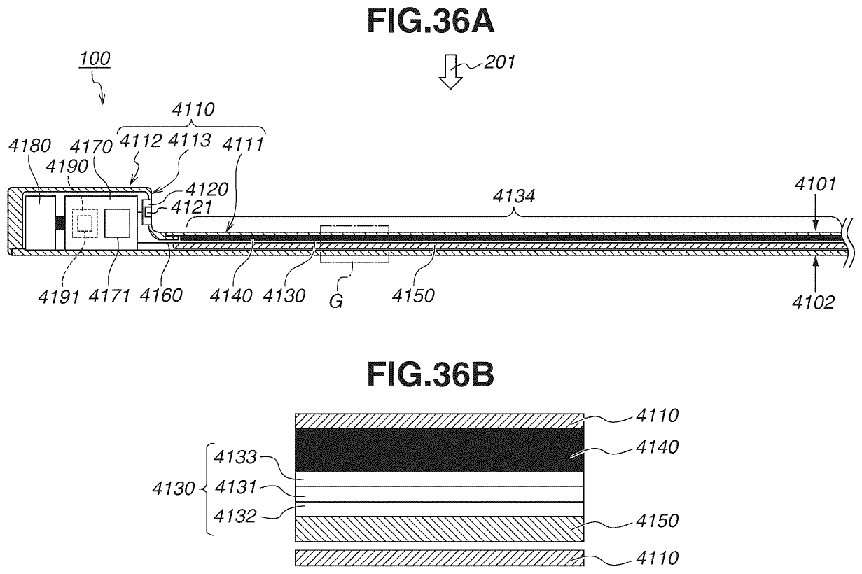

In FIG. 35, a housing 4110 of the radiographic imaging apparatus 100 is illustrated as the appearance of the radiographic imaging apparatus 100. An indicator 4114 indicating the range of an effective imaging area 4134 where a radiation detection panel (radiation detection panel 4130 of FIGS. 36A and 36B to be described below) accommodated in the housing 4110 detects the radiation 201 transmitted through the subject H is displayed on this housing 4110. In the example illustrated in FIG. 35, as can be seen from the indicator 4114 indicating the range of the effective imaging area 4134, the effective imaging area 4134 has a polygonal (specifically, rectangular) shape when seen from the side where the radiation 201 is incident.

As illustrated in FIG. 35, the housing 4110 includes a first thickness section 4111 that is a section including the effective imaging area 4134 and has a first thickness. As illustrated in FIG. 35, the housing 4110 also includes a second thickness section 4112 that is a section not including the effective imaging area 4134 and has a second thickness different from the thickness (first thickness) of the first thickness section 4111. Specifically, the thickness (second thickness) of the second thickness section 4112 is greater than the thickness (first thickness) of the first thickness section 4111. In such a case, the first thickness section 4111 may be referred to as a “thin section”, and the second thickness section 4112 may be referred to as a “thick section”. More specifically, in the example illustrated in FIG. 35, the second thickness section (thick section) 4112 is thicker than the first thickness section (thin section) 4111 toward the side where the radiation 201 is incident. As illustrated in FIG. 35, the housing 4110 further includes a connection section 4113 that connects the first thickness section 4111 and the second thickness section 4112.

The radiographic imaging apparatus 100 also includes a sensor unit 4120 on the side where the radiation 201 is incident on the housing 4110. The sensor unit 4120 includes one or more types of sensors for detecting the subject H. Specifically, the sensor unit 4120 is disposed on the housing 4110, outside at least one of the sides of the polygonal shape that is the shape of the effective imaging area 4134. More specifically, in the example illustrated in FIG. 35, the sensor unit 4120 is disposed on the connection section 4113, outside the side of the effective imaging area 4134 facing the second thickness section 4112.

FIGS. 36A and 36B are diagrams illustrating an example of an internal configuration in cross section F-F of the radiographic imaging apparatus 100 illustrated in FIG. 35. Specifically, FIG. 36A is a diagram illustrating an example of the internal configuration in cross section F-F of the radiographic imaging apparatus 100 illustrated in FIG. 35. FIG. 36B is an enlarged view of the region R illustrated in FIG. 36A. In these FIGS. 36A and 36B, components similar to those illustrated in FIG. 35 are denoted by the same reference numerals, and a detailed description thereof will be omitted.

As illustrated in FIG. 36A, the radiographic imaging apparatus 100 includes, in addition to the housing 4110 and the sensor unit 4120 illustrated in FIG. 35, a radiation detection panel 4130, a cushioning member 4140, a support base 4150, a flexible circuit board 4160, a control substrate 4170, a battery 4180, and a notification unit 4190.

In the example illustrated in FIG. 36A, the sensor unit 4120 is disposed on the connection section 4113 that connects the first thickness section 4111 and the second thickness section 4112 of the housing 4110 along a perpendicular. The sensor unit 4120 includes the one or more types of sensors 4121 for detecting the subject H.

The radiation detection panel 4130 is accommodated in the first thickness section 4111 of the housing 4110, and has the effective imaging area 4134 where the radiation 201 transmitted through the subject H is detected. As illustrated in FIG. 36B, this radiation detection panel 4130 includes a phosphor layer (scintillator layer) 4131, a sensor substrate 4132, and a phosphor protective film 4133. The phosphor layer (scintillator layer) 4131 converts the incident radiation 201 into light (such as visible light). The sensor substrate 4132 includes a plurality of photoelectric conversion elements that converts the light occurring in the phosphor layer (scintillator layer) 4131 into electrical signals related to a radiographic image. Here, the sensor substrate 4132 may be formed of materials such as glass and flexible plastic, whereas the present exemplary embodiment is not limited thereto. The phosphor protective film 4133 is disposed between the cushioning member 4140 and the phosphor layer (scintillator layer) 4131, formed of a material with low moisture permeability, and has a function of protecting the phosphor layer (scintillator layer) 4131. FIG. 36B illustrates an example of conversion elements of a so-called indirect conversion system, including the phosphor layer (scintillator layer) 4131 and the photoelectric conversion elements. However, for example, conversion elements of a direct conversion system where the incident radiation 201 is directly converted into electrical signals related to a radiographic image without providing the phosphor layer (scintillator layer) 4131 may be applied. If the conversion elements of this direct conversion system are applied, for example, a conversion element unit where conversion elements formed of a-Se or the like and electrical elements such as TFTs are two-dimensionally arranged may be constituted. However, this is not restrictive. The radiation detection panel 4130 includes the area of some or all of the plurality of photoelectric conversion elements formed on the sensor substrate 4132 as its effective imaging area 4134. This effective imaging area 4134 is an area that is capable of radiographic imaging and where radiographic images are actually generated on the radiation detection panel 4130. As illustrated in FIG. 35, the effective imaging area 4134 has a substantially rectangular shape when seen in the incident direction of the radiation 201. However, the present exemplary embodiment is not limited to the configuration illustrated in FIG. 35.

The cushioning member 4140 is accommodated in the first thickness section 4111 of the housing 4110 and disposed between the housing 4110 (radiation incident surface 4101) and the radiation detection panel 4130, and has a function of protecting the radiation detection panel 4130 from external force and the like. This cushioning member 4140 is suitably formed of materials such as a foamed resin and gel, but may be formed of other materials.

The support base 4150 is a base that is accommodated in the first thickness section 4111 of the housing 4110 and supports the radiation detection panel 4130 from a side with the rear surface 4102 of the radiographic imaging apparatus 100. This support base 4150 is suitably formed of materials with excellent lightweight properties, such as magnesium alloys, aluminum alloys, fiber-reinforced plastic, and plastic, but may be formed of other materials.

The flexible circuit board 4160 is connected to the radiation detection panel 4130 and the control substrate 4170. The flexible circuit board 4160 has functions such as reading the electrical signals related to a radiographic image (radiographic image signals) from the radiation detection panel 4130 and outputting the electrical signals to the control substrate 4170.

The control substrate 4170 is accommodated in the second thickness section 4112 of the housing 4110, controls the operation of the radiographic imaging apparatus 100 in a comprehensive manner, and performs various types of processing. For example, the control substrate 4170 processes the radiographic image signals output from the flexible circuit board 4160. For example, the control substrate 4170 performs processing for detecting the subject H based on detection result information about the subject H from the sensor unit 4120 (objects other than the subject H may also be detected). The control substrate 4170 includes a storage unit 4171 inside. The storage unit 4171 stores various types of information (including signals and data) needed when the control substrate 4170 performs various types of control and various types of processing, and programs needed when the control substrate 4170 performs various types of control and various types of processing. The storage unit 4171 also stores various types of information (including signals and data) obtained by the control substrate 4170 performing various types of control and various types of processing. In the example illustrated in FIG. 36A, the entire control substrate 4170 is accommodated in the second thickness section 4112 of the housing 4110. However, the control substrate 4170 may be configured to be accommodated in part in the second thickness section 4112 of the housing 4110.

The battery 4180 is accommodated in the second thickness section 4112 of the housing 4110, and supplies necessary power to the components of the radiographic imaging apparatus 100 via the control substrate 4170. Examples of the battery 4180 include a lithium-ion battery, an electric double layer capacitor, and an all-solid-state battery, whereas other batteries may be used.

The notification unit 4190 is disposed not in cross section F-F of the radiographic imaging apparatus 100 illustrated in FIG. 35 but on the far side or near side thereof, for example. The notification unit 4190 is accommodated in the second thickness section 4112 of the housing 4110, for example, and issues notification of the state of detection of the subject H by the control substrate 4170. For example, in a situation where a variation exceeding a predetermined level occurs in the subject H, the notification unit 4190 issues notification of the situation. Moreover, the notification unit 4190 includes a communication unit 4191 for communicating with external apparatuses such as a PC. The communication unit 4191 includes a wired communication unit using a cable or a wireless communication unit using a wireless LAN or the like, or both the wired communication unit and the wireless communication unit. For example, the communication unit 4191 transmits image data and the like on a radiographic image obtained by the radiographic imaging apparatus 100 to an external apparatus. The radiographic image is then displayed on a monitor or the like, and used for diagnosis etc. In the present exemplary embodiment, the notification unit 4190 notifies the user of the radiographic imaging apparatus 100 of the foregoing state of detection of the subject H, using speaker sound, LED or other display, or communication with an external apparatus via the communication unit 4191, for example.

To achieve portability and strength in a compatible manner, the housing 4110 is suitably formed of materials such as magnesium alloys, aluminum alloys, fiber-reinforced plastic, and other plastics, but may be formed of other materials. In particular, the radiation incident surface 4101 of the first thickness section 4111 including the effective imaging area 4134 is suitably formed of materials such as a carbon fiber-reinforced plastic with high transmittance for the radiation 201 and excellent lightweight properties, but may be formed of other materials.

When radiographing the subject H such as a patient, the radiographic imaging apparatus 100 may be placed immediately behind the imaging site of the subject H such as a patient. In doing so, a step created by the thickness of the housing 4110 of the radiographic imaging apparatus 100, the subject H such as a patient and the end portion of the housing 4110 of the radiographic imaging apparatus 100 come into contact to cause a reaction force, and the subject H such as a patient may feel discomfort. Typical radiographic imaging apparatuses are often configured in sizes complaint with ISO (International Organization for Standardization) 4090:2001, often with a thickness of approximately 15 mm to 16 mm. By contrast, in the radiographic imaging apparatus 100 according to the present exemplary embodiment, the first thickness section (thin section) 4111 of the housing 4110 has a thickness of 8.0 mm. With the radiographic imaging apparatus 100 according to the present exemplary embodiment, the step created by the thickness of the housing 4110 (first thickness section [thin section] 4111) is thus small, and the reaction force occurring between the subject H such as a patient and the end portion of the housing 4110 of the radiographic imaging apparatus 100 is reduced. To obtain this effect, the thickness of the first thickness section (thin section) 4111 does not need to be limited to 8.0 mm or so, and may be even smaller. Here, the applicant has confirmed that the foregoing effect is obtainable if the thickness of the housing 4110 (first thickness section [thin section] 4111) is less than 10.0 mm.

When radiographing the subject H such as a patient, the user such as a technician performs an operation of inserting the radiographic imaging apparatus 100 toward the imaging site of the subject H and positioning the radiographic imaging apparatus 100. During this operation, the subject H such as a patient and the radiographic imaging apparatus 100 may contact directly or via cloth or the like, such as towels and sheets. This cloth is often placed in view of reducing burden on the subject H such as a patient, maintaining hygiene, etc. In the present exemplary embodiment, as illustrated in FIGS. 35, 36A, and 36B, the sensor unit 4120 for detecting the subject H is thus disposed on the connection section 4113 of the housing 4110.

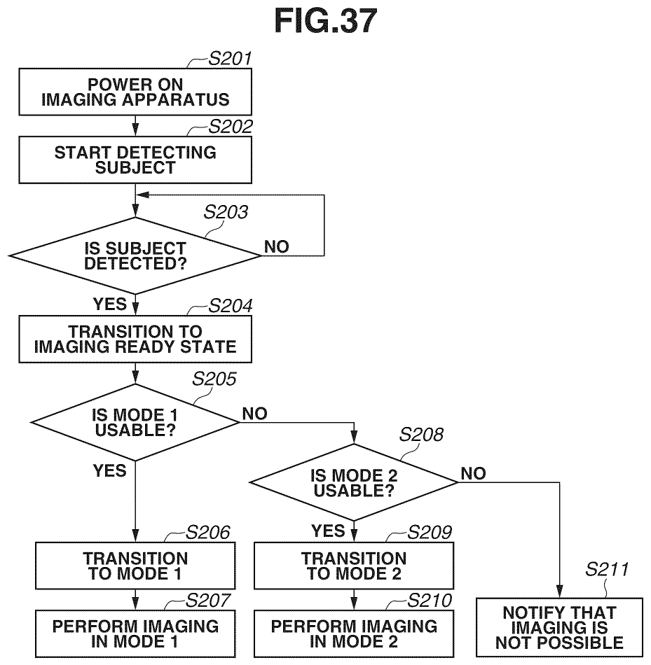

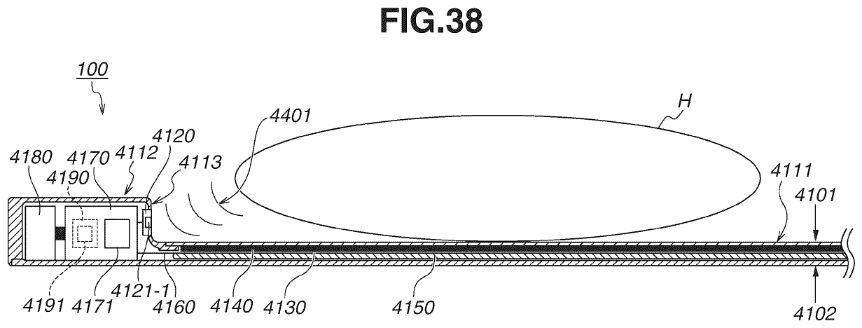

FIG. 37 is a flowchart illustrating an example of a processing procedure for a control method of the radiographic imaging apparatus 100 according to the thirteenth exemplary embodiment. FIG. 38 is a diagram illustrating an example of an internal configuration of the radiographic imaging apparatus 100 according to the thirteenth exemplary embodiment. Like FIG. 36A, this FIG. 38 is a diagram illustrating an example of the internal configuration in cross section F-F illustrated in FIG. 35. In this FIG. 38, components similar to those illustrated in FIGS. 35, 36A, and 36B are denoted by the same reference numerals, and a detailed description thereof will be omitted. Specifically, FIG. 38 illustrates an example where an infrared sensor 4121-1 to be used as a human detection sensor is applied as the sensor 4121 illustrated in FIGS. 36A and 36B. The flowchart illustrated in FIG. 37 will now be described with reference to the configuration illustrated in FIG. 38.

Initially, in step S201, when the radiographic imaging apparatus 100 is powered on, the control substrate 4170 supplies the power from the battery 4180 to the components of the radiographic imaging apparatus 100 and activates the radiographic imaging apparatus 100.

Next, in step S202, the control substrate 4170 starts detecting the subject H using the sensor unit 4120. With the detection operation of the subject H started, the sensor unit 4120 converts infrared information 4401 from the heat of the subject H into an electrical signal using the infrared sensor 4121-1, and transmits the electrical signal to the control substrate 4170 as detection result information about the subject H.

Next, in step S203, the control substrate 4170 determines whether the subject H is successfully detected, based on the detection result information from the sensor unit 4120. In the present exemplary embodiment, for example, if a signal change is detected in the detection result information (electrical signal) from the heat of the subject H, it is determined that the subject H is successfully detected on the effective imaging area 4134. For purposes such as preventing erroneous detection due to noise, for example, a threshold for the amount of signal change to determine that the subject H is successfully detected may be set and stored in the storage unit 4171 of the control substrate 4170 in advance.

If, as a result of the determination of step S203, the subject H is not successfully detected (NO in step S203), the processing waits in step S203 until the subject H is successfully detected.

On the other hand, if, as a result of the determination of step S203, the subject H is successfully detected (YES in step S203), the processing proceeds to step S204.

In step S204, the control substrate 4170 causes the radiographic imaging apparatus 100 to transition to an imaging ready state.

Here, the radiographic imaging apparatus 100 according to the present exemplary embodiment has a plurality of imaging modes for the radiographic imaging of the subject H. The radiographic imaging apparatus 100 according to the present exemplary embodiment then stores information indicating the use order of the plurality of imaging modes in the storage unit 4171 in advance, and may determine the imaging mode to transition to based on whether the imaging modes are usable. Here, in the present exemplary embodiment, the plurality of imaging modes shall include imaging mode 1 and imaging mode 2. Here, imaging mode 1 refers to the imaging mode where the information indicates the highest use order among the plurality of imaging modes. In other words, imaging mode 1 is an imaging mode where the information indicating the use order is high compared to imaging mode 2. An example of imaging mode 1 is a synchronous mode where the radiographic imaging apparatus 100 communicates with the radiation generation apparatus 200 and performs radiographic imaging in synchronization with the radiation generation apparatus 200.

An example of imaging mode 2 is an automatic mode where the radiographic imaging apparatus 100 is not synchronized with the radiation generation apparatus 200, and the radiographic imaging apparatus 100 detects exposure to the radiation 201 and automatically performs radiographic imaging. While the two imaging modes, namely, imaging mode 1 and imaging mode 2 are described here, any number of usable imaging modes may be set.

If the processing of step S204 is completed, the processing proceeds to step S205.

In step S205, based on the information indicating the use order stored in the storage unit 4171, the control substrate 4170 determines whether imaging mode 1 is usable depending on whether synchronization is able to be established through communication with the radiation generation apparatus 200.

If, as a result of the determination of step S205, imaging mode 1 is usable (YES in step S205), the processing proceeds to step S206.

In step S206, the control substrate 4170 sets the imaging mode for the radiographic imaging of the subject H to imaging mode 1, and causes the radiographic imaging apparatus 100 to transition to imaging mode 1.

Next, in step S207, the control substrate 4170 performs the radiographic imaging of the subject H in imaging mode 1.

If, as a result of the determination of step S205, imaging mode 1 is not usable (NO in step S205), the processing proceeds to step S208.

In step S208, based on the information indicating the use order stored in the storage unit 4171, the control substrate 4170 determines whether imaging mode 2 is usable with the radiographic imaging apparatus 100.

If, as a result of the determination of step S208, imaging mode 2 is usable (YES in step S208), the processing proceeds to step S209.

In step S209, the control substrate 4170 sets the imaging mode for the radiographic imaging of the subject H to imaging mode 2, and causes the radiographic imaging apparatus 100 to transition to imaging mode 2.

Next, in step S210, the control substrate 4170 performs the radiographic imaging of the subject H in imaging mode 2.

If, as a result of the determination of step S208, imaging mode 2 is not usable (NO in step S208), the processing proceeds to step S211.

In step S211, the control substrate 4170 causes the notification unit 4190 to notify the user that the radiographic imaging is not possible. Here, the notification unit 4190 notifies the user of the radiographic imaging apparatus 100 that the imaging is not possible, using speaker sound, LED or other display, or communication with an external apparatus via the communication unit 4191, for example.

If the processing of step S207 is completed, if the processing of step S210 is completed, or if the processing of step S211 is completed, the processing of the flowchart of FIG. 37 ends.

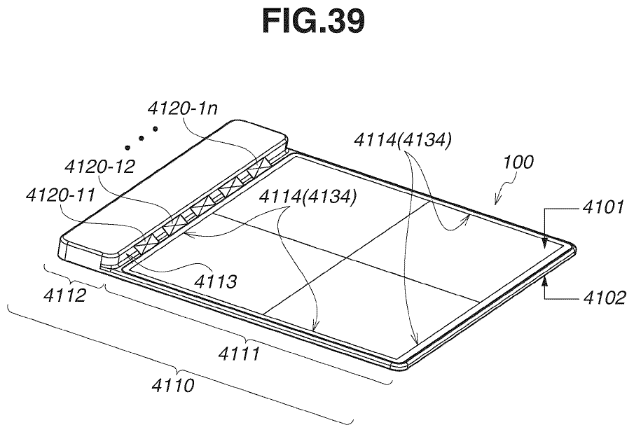

FIG. 39 is a diagram illustrating modification 1 of the schematic configuration of the radiographic imaging apparatus 100 according to the thirteenth exemplary embodiment. In this FIG. 39, components similar to those illustrated in FIGS. 35, 36A, 36B, and 38 are denoted by the same reference numerals, and a detailed description thereof will be omitted.

Specifically, the radiographic imaging apparatus 100 illustrated in FIG. 39 differs from FIG. 35 in that a plurality of (n) sensor units 4120-11 to 4120-1n is disposed on the connection section 4113, outside the side of the effective imaging area 4134 facing the second thickness section 4112. The radiographic imaging apparatus 100 illustrated in this FIG. 39 may be configured so that a sensor unit 4120 to be used is selected from the plurality of (n) sensor units 4120-11 to 4120-1n.

Whether the subject H is successfully detected may be determined by combining pieces of detection result information from a plurality of sensor units 4120.

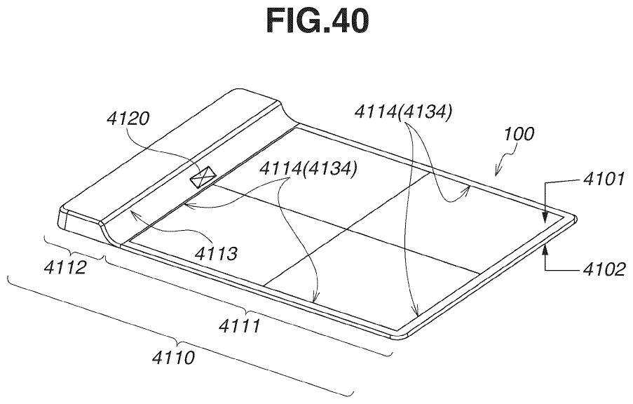

FIG. 40 is a diagram illustrating modification 2 of the schematic configuration of the radiographic imaging apparatus 100 according to the thirteenth exemplary embodiment. In this FIG. 40, components similar to those illustrated in FIGS. 35, 36A, 36B, 38, and 39 are denoted by the same reference numerals, and a detailed description thereof will be omitted.

Specifically, the radiographic imaging apparatus 100 illustrated in FIG. 40 differs from FIG. 35 and the like in the shape of the connection section 4113 where the sensor unit 4120 is disposed. More specifically, the connection section illustrated in FIG. 40 is a sloped surface connecting the first thickness section 4111 and the second thickness section 4112 of the housing 4110 along an oblique line.

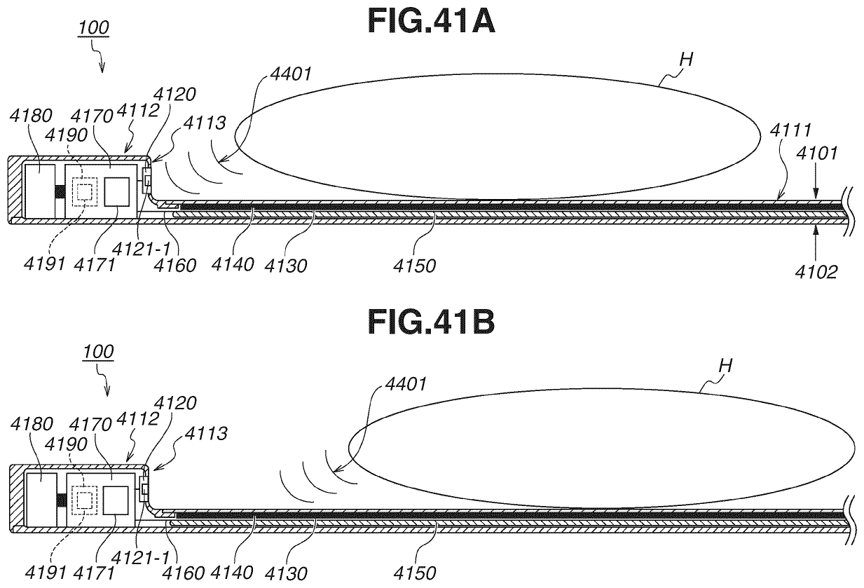

The subject H may move during the period between the transition to a usable imaging mode and the actual radiographic imaging. A case where the subject H moves will be described with reference to FIGS. 41A and 41B.

FIGS. 41A and 41B are diagrams illustrating an example of the internal configuration of the radiographic imaging apparatus 100 according to the thirteenth exemplary embodiment. In these FIGS. 41A and 41B, components similar to those illustrated in FIG. 38 are denoted by the same reference numerals, and a detailed description thereof will be omitted.

If the subject H moves from the state illustrated in FIG. 41A to the state illustrated in FIG. 41B, the subject H moves in a direction away from the sensor unit 4120. In such a case, the infrared information 4401 from the heat of the subject H reaching the sensor unit 4120 decreases, and a signal decrease occurs in the detection result information (electrical signal) from the sensor unit 4120 as well. On the other hand, if the subject H moves from the state illustrated in FIG. 41B to the state illustrated in FIG. 41A, the subject H moves in a direction toward the sensor unit 4120, and as a result, a signal increase occurs in the detection result information (electrical signal) from the sensor unit 4120 as well. When a certain change occurs thus in the detection result information (electrical signal) from the sensor unit 4120, the notification unit 4190 may notify the user of the radiographic imaging apparatus 100 that a variation has occurred in the subject H. Here, the change (increase or decrease) in the detection result information (electrical signal) and the amount of change to issue the notification are desirably determined in advance and stored in the storage unit 4171 of the control substrate 4170. The user can move the subject H to an appropriate position by adjusting the position and the like of the subject H based on the information notified from the notification unit 4190.

The radiographic imaging apparatus 100 according to the thirteenth exemplary embodiment described above includes the radiation detection panel 4130 including the effective imaging area 4134 where the radiation 201 transmitted through the subject H is detected. The radiographic imaging apparatus 100 according to the thirteenth exemplary embodiment also includes the housing 4110 that accommodates the radiation detection panel 4130 and where the effective imaging area 4134 has a polygonal shape as seen from the side where the radiation 201 is incident. The radiographic imaging apparatus 100 according to the thirteenth exemplary embodiment further includes the sensor unit 4120 that is disposed on the housing 4110, outside at least one side of the polygonal shape that is the shape of the effective imaging area 4134, and includes at least one or more types of sensors 4121 for detecting the subject H.

With such a configuration of the radiographic imaging apparatus 100, whether there is a subject H on the effective imaging area 4134 is detected, for example. This can improve the user's workability during radiographic imaging and enables quick radiographic imaging.

### Fourteenth Exemplary Embodiment

Next, a fourteenth exemplary embodiment will be described. In the following description of the fourteenth exemplary embodiment, a description of items common to the foregoing thirteenth exemplary embodiment is omitted, and differences from the foregoing thirteenth exemplary embodiment will mainly be described.

A radiographic imaging system 10 according to the fourteenth exemplary embodiment has a schematic configuration similar to that of the radiographic imaging system 10 according to the thirteenth exemplary embodiment illustrated in FIG. 35.

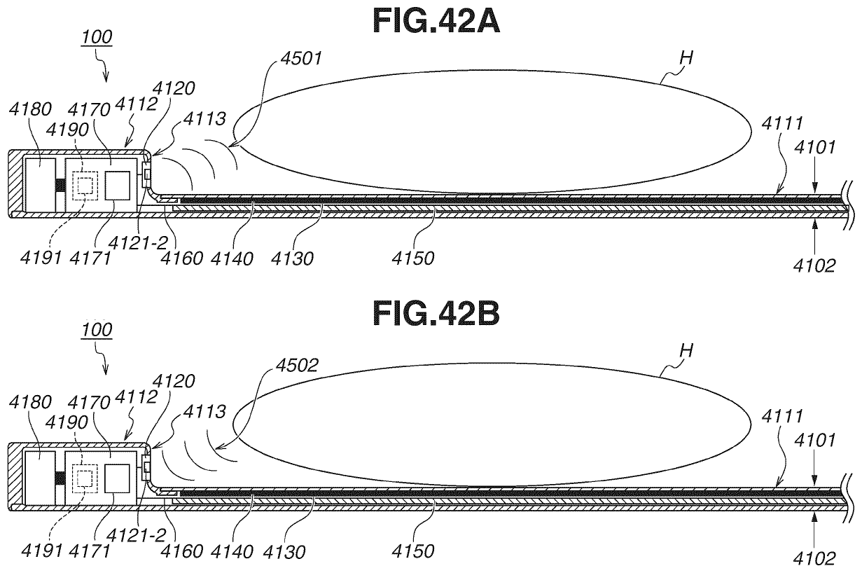

FIGS. 42A and 42B are diagrams illustrating an example of an internal configuration of a radiographic imaging apparatus 100 according to the fourteenth exemplary embodiment. In these FIGS. 42A and 42B, components similar to those illustrated in FIGS. 35, 36A, 36B, and 38 to 41A and 41B are denoted by the same reference numerals, and a detailed description thereof will be omitted.

The radiographic imaging apparatus 100 according to the thirteenth exemplary embodiment is configured so that the infrared sensor 4121-1 is applied as the sensor 4121 included in the sensor unit 4120. By contrast, as illustrated in FIGS. 42A and 42B, the radiographic imaging apparatus 100 according to the fourteenth exemplary embodiment is configured so that an ultrasonic sensor 4121-2 is applied as the sensor 4121 included in the sensor unit 4120. The ultrasonic sensor 4121-2 may use the same sensor to both transmit ultrasound toward the subject H and receive ultrasound reflected from the subject H. A transmission ultrasonic sensor and a reception ultrasonic sensor may be separately disposed.

The fourteenth exemplary embodiment is configured so that when the detection operation of the subject H is started in step S202 in the flowchart of FIG. 37, the ultrasonic sensor 4121-2 included in the sensor unit 4120 transmits ultrasound toward the effective imaging area 4134 and receives reflection of the ultrasound.

Specifically, as illustrated in FIG. 42A, the ultrasonic sensor 4121-2 included in the sensor unit 4120 transmits ultrasonic transmission waves 4501 toward the subject H on the effective imaging area 4134. As illustrated in FIG. 42B, the ultrasonic sensor 4121-2 included in the sensor unit 4120 then receives ultrasonic reflection waves 4502 reflected by the subject H. The strength of the ultrasonic transmission waves 4501 and the transmission and reception intervals of the ultrasound are desirably set to certain values and stored in the storage unit 4171 of the control substrate 4170 in advance. The sensor unit 4120 then converts the received ultrasonic reflection waves 4502 into an electrical signal and transmits the electrical signal to the control substrate 4170 as detection result information about the subject H.

Next, in step S203 of FIG. 37, if a signal change in the ultrasonic reflection waves 4502 due to the placement of the subject H on the effective imaging area 4134 is detected based on the detection result information from the sensor unit 4120, the control substrate 4170 determines that the subject H is successfully detected. For purposes such as preventing erroneous detection due to noise, a threshold for the amount of signal change to determine that the subject H is successfully detected may be set and stored in the storage unit 4171 of the control substrate 4170 in advance.

If, as a result of the determination of step S203 in FIG. 37, the subject H is successfully detected (YES in step S203), the processing proceeds to step S204. In step S204, the control substrate 4170 causes the radiographic imaging apparatus 100 to transition to an imaging ready state. The processing of steps S205 onward in FIG. 37 is then performed.

Even in the present exemplary embodiment, the subject H may move during the period between the transition to a usable imaging mode and the actual radiographic imaging. If the subject H moves in a direction away from the sensor unit 4120, the ultrasonic reflection waves 5402 reaching the sensor unit 4120 decrease, and a signal decrease occurs in the detection result information (electrical signal) from the sensor unit 4120 as well. On the other hand, if the subject H moves in a direction toward the sensor unit 4120, the ultrasonic reflection waves 4502 reaching the sensor unit 4120 increase, and a signal increase occurs in the detection result information (electrical signal) from the sensor unit 4120 as well. When a certain change occurs thus in the detection result information (electrical signal) from the sensor unit 4120, the notification unit 4190 may notify the user of the radiographic imaging apparatus 100 that a variation has occurred in the subject H. Here, the change (increase or decrease) in the detection result information (electrical signal) and the amount of change to issue the notification are desirably determined in advance and stored in the storage unit 4171 of the control substrate 4170. The user can move the subject H to an appropriate position by adjusting the position and the like of the subject H based on the information notified from the notification unit 4190.

In the present exemplary embodiment, the infrared sensor 4121-1 applied in the thirteenth exemplary embodiment may be disposed in the sensor unit 4120 along with the ultrasonic sensor 4121-2. In such a case, the sensor unit 4120 may use the ultrasonic sensor 4121-2 and the infrared sensor 4121-1 in combination.

The fourteenth exemplary embodiment, like the foregoing thirteenth exemplary embodiment, also improves the user's workability during radiographic imaging and enables quick radiographic imaging.

### Fifteenth Exemplary Embodiment

Next, a fifteenth exemplary embodiment will be described. In the following description of the fifteenth exemplary embodiment, a description of items common to the foregoing thirteenth and fourteenth exemplary embodiments is omitted, and differences from the foregoing thirteenth and fourteenth exemplary embodiments will mainly be described.

A radiographic imaging system 10 according to the fifteenth exemplary embodiment has a schematic configuration similar to that of the radiographic imaging system 10 according to the thirteenth exemplary embodiment illustrated in FIG. 35.

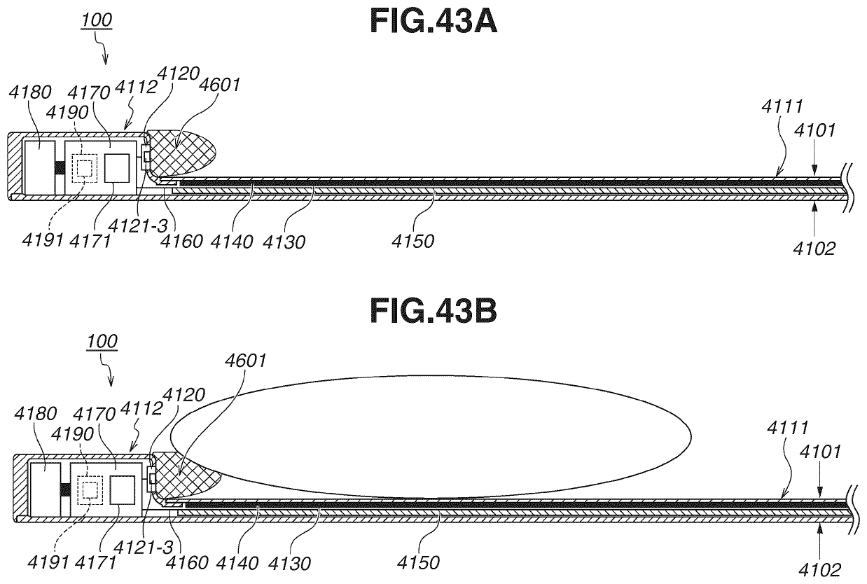

FIGS. 43A and 43B are diagrams illustrating an example of an internal configuration of a radiographic imaging apparatus 100 according to the fifteenth exemplary embodiment. In these FIGS. 43A and 43B, components similar to those illustrated in FIGS. 35, 36A, 36B, and 38 to 42A and 42B are denoted by the same reference numerals, and a detailed description thereof will be omitted.

The radiographic imaging apparatus 100 according to the thirteenth exemplary embodiment is configured so that the infrared sensor 4121-1 is applied as the sensor 4121 included in the sensor unit 4120. By contrast, as illustrated in FIGS. 43A and 43B, the radiographic imaging apparatus 100 according to the fifteenth exemplary embodiment is configured so that a capacitive sensor 4121-3, which is suitably used as a touch sensor and the like, is applied as the sensor 4121 included in the sensor unit 4120. As illustrated in FIGS. 43A and 43B, the capacitive sensor 4121-3 generates an electric field region 4601. As illustrated in FIG. 43B, when the subject H enters the electric field region 4601 generated by the capacitive sensor 4121-3, the control substrate 4170 detects a change in capacitance due to a change in the electric field and thereby detects the subject H.

In the fifteenth exemplary embodiment, when the detection operation of the subject H is started in step S202 in the flowchart of FIG. 37, the capacitive sensor 4121-3 included in the sensor unit 4120 generates the electric field region 4601. The strength of the electric field region 4601 is desirably stored in the storage unit 4171 of the control substrate 4170 in advance. The sensor unit 4120 then converts a change in capacitance due to a change in the electric field of the electric field region 4601 into an electrical signal, and transmits the electrical signal to the control substrate 4170 as detection result information about the subject H.

Next, in step S203 of FIG. 37, if a change in capacitance due to the placement of the subject H on the effective imaging area 4134 is detected based on the detection result information from the sensor unit 4120, the control substrate 4170 determines that the subject H is successfully detected. For purposes such as preventing erroneous detection due to noise, a threshold for the amount of signal change to determine that the subject H is successfully detected may be set and stored in the storage unit 4171 of the control substrate 4170 in advance.

If, as a result of the determination of step S203 in FIG. 37, the subject H is successfully detected (YES in step S203), the processing proceeds to step S204. In step S204, the control substrate 4170 causes the radiographic imaging apparatus 100 to transition to an imaging ready state. The processing of steps S205 onward in FIG. 37 is then performed.

Even in the present exemplary embodiment, the subject H may move during the period between the transition to a usable imaging mode and the actual radiographic imaging. If the subject H moves in a direction away from the sensor unit 4120, the capacitance detected by the sensor unit 4120 restores the state where there is no subject H in the electric field region 4601. On the other hand, if the subject H moves in a direction toward the sensor unit 4120, the capacitance detected by the sensor unit 4120 changes further. In such a case, the notification unit 4190 may notify the user of the radiographic imaging apparatus 100 that a variation has occurred in the subject H. Here, the change in the detection result information (electrical signal) and the amount of change to issue the notification are desirably determined in advance and stored in the storage unit 4171 of the control substrate 4170. The user can move the subject H to an appropriate position by adjusting the position and the like of the subject H based on the information notified from the notification unit 4190.

In the present exemplary embodiment, at least one sensor 4121 between the infrared sensor 4121-1 and the ultrasonic sensor 4121-2 applied in the thirteenth and fourteenth exemplary embodiments may be disposed inside the sensor unit 4120 along with the capacitive sensor 4121-3. In such a case, the sensor unit 4120 may use the capacitive sensor 4121-3 and the at least one sensor 4121 between the infrared sensor 4121-1 and the ultrasonic sensor 4121-2 in combination.

The fifteenth exemplary embodiment, like the foregoing thirteenth exemplary embodiment, also improves the user's workability during radiographic imaging and enables quick radiographic imaging.

### Sixteenth Exemplary Embodiment

Next, a sixteenth exemplary embodiment will be described. In the following description of the sixteenth exemplary embodiment, a description of items common to the foregoing thirteenth to fifteenth exemplary embodiments is omitted, and differences from the foregoing thirteenth to fifteenth exemplary embodiments will mainly be described.

A radiographic imaging system 10 according to the sixteenth exemplary embodiment has a schematic configuration similar to that of the radiographic imaging system 10 according to the thirteenth exemplary embodiment illustrated in FIG. 35.

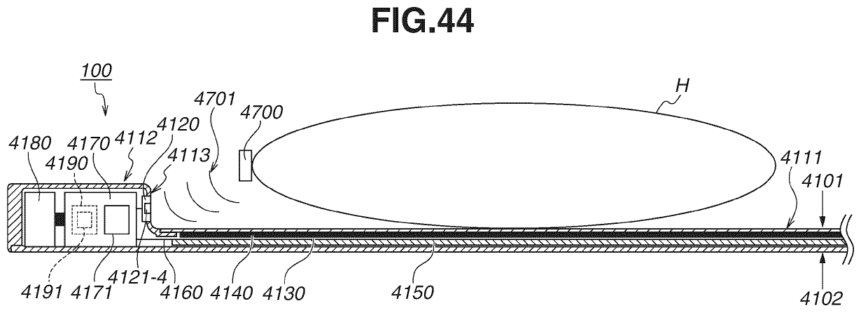

FIG. 44 is a diagram illustrating an example of an internal configuration of a radiographic imaging apparatus 100 according to the sixteenth exemplary embodiment. In this FIG. 44, components similar to those illustrated in FIGS. 35, 36A, 36B, and 38 to 43A and 43B are denoted by the same reference numerals, and a detailed description thereof will be omitted.

The radiographic imaging apparatus 100 according to the thirteenth exemplary embodiment is configured so that the infrared sensor 4121-1 is applied as the sensor 4121 included in the sensor unit 4120. By contrast, as illustrated in FIG. 44, the radiographic imaging apparatus 100 according to the sixteenth exemplary embodiment is configured so that a magnetic sensor 4121-4 is applied as the sensor 4121 included in the sensor unit 4120. In the case of the sixteenth exemplary embodiment, as illustrated in FIG. 44, a magnetic marker 4700 is attached to near the imaging site of the subject H in advance. When the magnetic marker 4700 attached to the subject H approaches the sensor unit 4120, the control substrate 4170 detects a change in a magnetic field 4701 detected by the magnetic sensor 4121-4 and thereby detects the subject H

In the sixteenth exemplary embodiment, when the detection operation of the subject H is started in step S202 in the flowchart of FIG. 37, the following processing is performed. Specifically, the sensor unit 4120 converts a change in the magnetic field 4701 detected by the magnetic sensor 4121-4 into an electrical signal, and transmits the electrical signal to the control substrate 4170 as detection result information about the subject H.

Next, in step S203 of FIG. 37, the control substrate 4170 performs the following determination based on the detection result information from the sensor unit 4120. That is, if a change in the magnetic field 4710 due to the approach of the magnetic marker 4700 to the sensor unit 4120 and the placement of the subject H on the effective imaging area 4134 is detected, the control substrate 4170 determines that the subject H is successfully detected. For purposes such as preventing erroneous detection due to noise, a threshold for the amount of signal change to determine that the subject H is successfully detected may be set and stored in the storage unit 4171 of the control substrate 4170 in advance. As for the threshold setting, the strength of the magnetic field 4701 and the amount of change therein when the magnetic marker 4700 approaches the sensor unit 4120 within a desired distance may be measured in advance, and the threshold may be set based on the measurements.

If, as a result of the determination of step S203 in FIG. 37, the subject H is successfully detected (YES in step S203), the processing proceeds to step S204. In step S204, the control substrate 4170 causes the radiographic imaging apparatus 100 to transition to an imaging ready state. The processing of steps S205 onward in FIG. 37 is then performed.

Even in the present exemplary embodiment, the subject H may move during the period between the transition to a usable imaging mode and the actual radiographic imaging. If the magnetic marker 4700 attached to the subject H moves in a direction away from the sensor unit 4120, the strength of the magnetic field 4701 detected by the magnetic sensor 4121-4 decreases, and a signal decrease occurs in the detection result information (electrical signal) from the sensor unit 4120 as well. On the other hand, if the magnetic marker 4700 attached to the subject H moves in a direction toward the sensor unit 4120, a signal increase occurs in the detection result information (electrical signal) from the sensor unit 4120. When a certain change occurs thus in the detection result information (electrical signal) from the sensor unit 4120, the notification unit 4190 may notify the user of the radiographic imaging apparatus 100 that a variation has occurred in the subject H. Here, the change in the detection result information (electrical signal) and the amount of change to issue the notification is desirably determined in advance and stored in the storage unit 4171 of the control substrate 4170. The user can move the subject H to an appropriate position by adjusting the position and the like of the subject H based on the information notified from the notification unit 4190.

In the present exemplary embodiment, the magnetic sensor 4121-4 and at least one sensor 4121 among the infrared sensor 4121-1, the ultrasonic sensor 4121-2, and the capacitive sensor 4121-3 applied in the thirteenth to fifteenth exemplary embodiments may be disposed inside the sensor unit 4120. In such a case, the sensor unit 4120 may use the magnetic sensor 4121-4 and the at least one sensor 4121 among the infrared sensor 4121-1, the ultrasonic sensor 4121-2, and the capacitive sensor 4121-3 applied in the thirteenth to fifteenth exemplary embodiments in combination.

The sixteenth exemplary embodiment, like the foregoing thirteenth exemplary embodiment, also improves the user's workability during radiographic imaging and enables quick radiographic imaging.

### Seventeenth Exemplary Embodiment

Next, a seventeenth exemplary embodiment will be described. In the following description of the seventeenth exemplary embodiment, a description of items common to the foregoing thirteenth to sixteenth exemplary embodiments is omitted, and differences from the foregoing thirteenth to sixteenth exemplary embodiments will mainly be described.

A radiographic imaging system 10 according to the seventeenth exemplary embodiment has a schematic configuration similar to that of the radiographic imaging system 10 according to the thirteenth exemplary embodiment illustrated in FIG. 35.

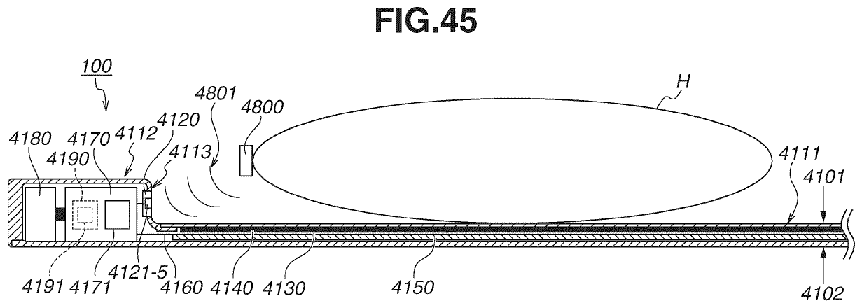

FIG. 45 is a diagram illustrating an example of an internal configuration of a radiographic imaging apparatus 100 according to the seventeenth exemplary embodiment. In this FIG. 45, components similar to those illustrated in FIGS. 35, 36A, 36B, and 38 to 44 are denoted by the same reference numerals, and a detailed description thereof will be omitted.

The radiographic imaging apparatus 100 according to thirteenth exemplary embodiment is configured so that the infrared sensor 4121-1 is applied as the sensor 4121 included in the sensor unit 4120. By contrast, as illustrated in FIG. 45, the radiographic imaging apparatus 100 according to the seventeenth exemplary embodiment is configured so that a proximity wireless sensor 4121-5, which is suitably used for individual identification as an RFID or the like, is applied as the sensor 4121 included in the sensor unit 4120. In the case of the seventeenth exemplary embodiment, as illustrated in FIG. 45, an RF tag 4800 is attached to near the imaging site of the subject H in advance.

In the seventeenth exemplary embodiment, when the detection operation of the subject H is started in step S202 in the flowchart of FIG. 37, the proximity wireless sensor 4121-5 included in the sensor unit 4120 transmits radio waves to detect the RF tag 4800. When the RF tag 4800 attached to the subject H approaches the sensor unit 4120, the RF tag 4800 adds ID information to the radio waves (transmission waves) transmitted from the proximity wireless sensor 4121-5 and returns the resulting radio waves 4801 to the sensor unit 4120. The sensor unit 4120 then detects the ID information from the radio waves 4801 received by the proximity wireless sensor 4121-5, and transmits the ID information to the control substrate 4170 as detection result information about the subject H.

A plurality of RF tags 4800 may be prepared in advance and ID information may be stored in the storage unit 4171 of the control substrate 4170 so that only a desired tag is detected as the subject H.

In the present exemplary embodiment, the RF tag 4800 is described to be a so-called passible tag that returns the radio waves 4801 obtained by adding ID information to the transmission waves. However, the RF tag 4800 may include a built-in battery and actively transmit radio waves including ID information to the sensor unit 4120. In such a case, the proximity wireless sensor 4121-5 included in the sensor unit 4120 performs only reception without transmitting radio wave.

Next, in step S203 of FIG. 37, the control substrate 4170 determines that the subject H is successfully detected on the effective imaging area 4134 based on the detection result information from the sensor unit 4120.

If, as a result of the determination of step S203 in FIG. 37, the subject H is successfully detected (YES in step S203), the processing proceeds to step S204. In step S204, the control substrate 4170 causes the radiographic imaging apparatus 100 to transition to an imaging ready state. The processing of steps S205 onward in FIG. 37 is then performed.

Even in the present exemplary embodiment, the subject H may move during the period between the transition to a usable imaging mode and the actual radiographic imaging. If the RF tag 4800 attached to the subject H moves in a direction away from the sensor unit 4120, the ID information of the RF tag 4800 is no longer read. In such a case, the notification unit 4190 may notify the user of the radiographic imaging apparatus 100 that a variation has occurred in the subject H. The user can move the subject H to an appropriate position by adjusting the position and the like of the subject H based on the information notified from the notification unit 4190.

In the present exemplary embodiment, the proximity wireless sensor 4121-5 and at least one sensor 4121 among the sensors 4121-1 to 4121-4 applied in the thirteenth to sixteenth exemplary embodiments may be disposed inside the sensor unit 4120. In such a case, the sensor unit 4120 may use the magnetic sensor 4121-5 and the at least one sensor 4121 among the sensors 4121-1 to 4121-4 applied in the thirteenth to sixteenth exemplary embodiments in combination.

The seventeenth exemplary embodiment, like the foregoing thirteenth exemplary embodiment, also improves the user's workability during radiographic imaging and enables quick radiographic imaging.

### Eighteenth Exemplary Embodiment

Next, an eighteenth exemplary embodiment will be described. In the following description of the eighteenth exemplary embodiment, a description of items common to the foregoing thirteenth to seventeenth exemplary embodiments is omitted, and differences from the foregoing thirteenth to seventeenth exemplary embodiments will mainly be described.

In the foregoing thirteenth to seventeenth exemplary embodiments, the use methods of various sensors that may be used to detect the subject H have been described. It is then conceivable to use various sensors in combination to identify whether a detected object is the subject H or an object other than the subject H. The eighteenth exemplary embodiment deals with a mode where the sensors 4121-1 to 4121-5 described in the thirteenth to seventeenth exemplary embodiment are used in combination to identify whether a detected object is the subject H or an object other than the subject H.

For example, when radiographing a patient as the subject H, the user such as a technician performs operation of inserting a radiographic imaging apparatus 100 toward the imaging site of the subject H such as a patient and adjusting the position of the radiographic imaging apparatus 100. During this operation, the subject H such as a patient and the radiographic imaging apparatus 100 may contact directly or via cloth or the like such as towels and sheets. This cloth is often placed in view of reducing burden on the subject H such as a patient and maintaining hygiene, etc. When towels, sheets, or the like are used, the presence of a subject H may be erroneously detected even with only the towels or sheets present.

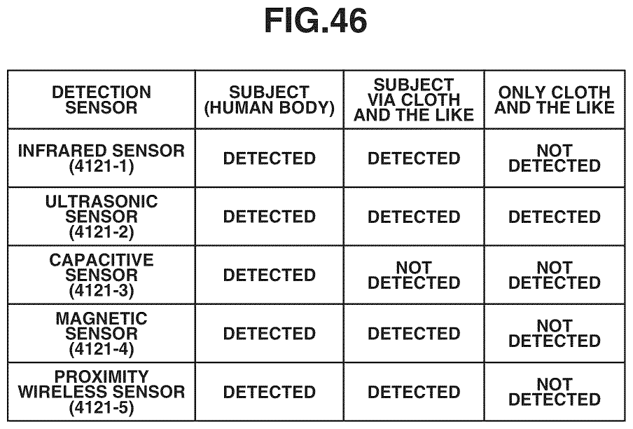

FIG. 46 is a chart illustrating an example of detection capabilities of the sensors 4121-1 to 4121-5 applied in the thirteenth to seventeenth exemplary embodiments. Specifically, FIG. 46 illustrates an example of the detection capabilities of the sensors 4121-1 to 4121-5 applied in the thirteenth to seventeenth exemplary embodiments for a subject (human body) H, a human body via cloth or the like, and cloth or the like alone.

The infrared sensor 4121-1 detects infrared rays from to the heat of the subject H, and is thus able to detect the subject H via cloth and the like as illustrated in FIG. 46. However, the infrared sensor 4121-1 is not capable of making a distinction between only the subject H and via cloth etc.

The magnetic sensor 4121-4 and the proximity wireless sensor 4121-5 detect the magnetic marker 4700 and the RF tag 4800 attached to the subject H, and is thus able to detect the subject H via cloth and the like as illustrated in FIG. 46. However, the magnetic sensor 4121-4 and the proximity wireless sensor 4121-5 are not capable of making a distinction between only the subject H and via cloth etc.

As illustrated in FIG. 46, the capacitive sensor 4121-3 does not detect cloth or the like, and may be unable to detect the subject H via cloth and the like.

The ultrasonic sensor 4121-2 detects the presence of any object that reflects the ultrasound, and may therefore be able to detect the presence of only cloth and the like as illustrated in FIG. 46

A method for identifying whether the detected object is the subject H, the subject H via cloth and the like, or only cloth and the like using differences in the foregoing detection capabilities of the sensors 4121-1 to 4121-5 is conceivable. In the present exemplary embodiment, a mode where the sensor unit 4120 includes the infrared sensor 4121-1, the ultrasonic sensor 4121-2, and the capacitive sensor 4121-3 and these sensors 4121-1 to 4121-3 are combined will be described. Note that the present invention is not limited to the combination of the sensors 4121 described in the present exemplary embodiment, and any plurality of sensors 4121 may be applied in combination.

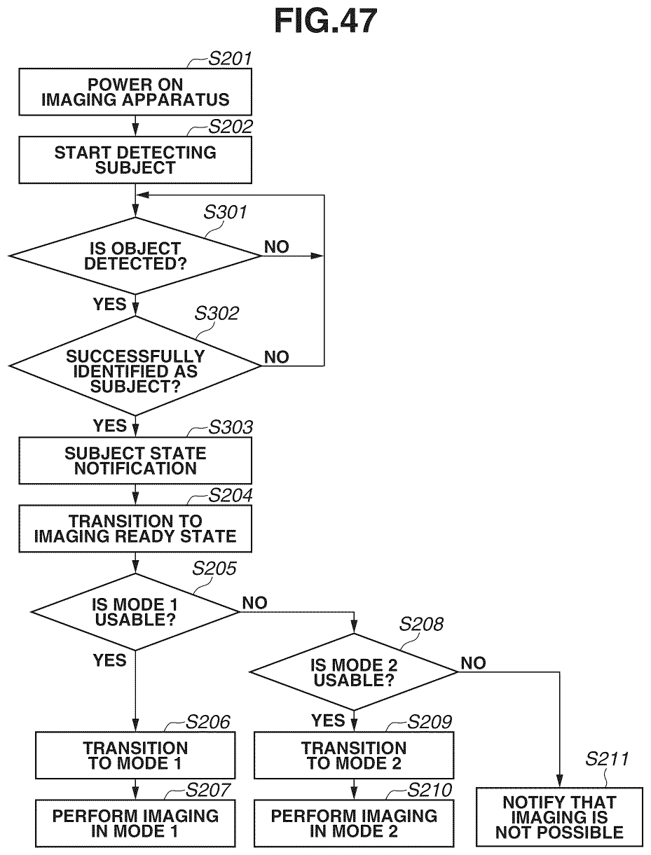

FIG. 47 is a flowchart illustrating an example of a processing procedure for a control method of the radiographic imaging apparatus 100 according to the eighteenth exemplary embodiment. In this FIG. 47, processing steps similar to those illustrated in FIG. 37 are denoted by the same step numbers, and a detailed description thereof will be omitted.

Initially, in step S201 of FIG. 47, when the radiographic imaging apparatus 100 is powered on, the control substrate 4170 supplies the power from the battery 4180 to the components of the radiographic imaging apparatus 100 and activates the radiographic imaging apparatus 100.

Next, in step S202 of FIG. 47, the control substrate 4170 starts detecting the subject H using the sensor unit 4120. Specifically, in the present exemplary embodiment, the control substrate 4170 performs detection using each of the infrared sensor 4121-1, the ultrasonic sensor 4121-2, and the capacitive sensor 4121-3 included in the sensor unit 4120.

Next, in step S301, the control substrate 4170 determines whether an object is detected by any of the sensors 4121 among the sensors 4121-1 to 4121-3. If, as a result of this determination, an object is not successfully detected by any of the sensors 4121 among the sensors 4121-1 to 4121-3 (NO in step S310), the processing waits in step S301 until an object is detected by one of the sensors 4121.

If, as a result of the determination of step S301, an object is detected by one of the sensors 4121 among the sensors 4121-1 to 4121-3 (YES in step S301), the processing proceeds to step S302.

In step S302, the control substrate 4170 determines whether the subject detected by at least one of the sensors is able to be identified as the subject H. The identification conditions about the subject H are desirably determined in view of the characteristics of the sensors 4121 in advance and stored in the storage unit 4171 of the control substrate 4170. For example, from the characteristics illustrated in FIG. 46, the object may be identified as the subject H if the object is successfully detected by two or more of the sensors 4121 among the infrared sensor 4121-1, the ultrasonic sensor 4121-2, and the capacitive sensor 4121-3. This can prevent erroneous detection of cloth and the like by the ultrasonic sensor 4121-2.

If, as a result of the determination of step S302, the objected detected by at least one of the sensors is not successfully identified as the subject H (NO in step S302), the processing proceeds to step S301. Here, the control substrate 4170 may cause the notification unit 4190 to notify the user that the object is not identified as the subject H. In such a case, since the notification is considered to last until the subject H is successfully identified, the notification unit 4190 desirably uses a notification method or means that does not interfere with the user's operation, such as display on the display unit.

If, as a result of the determination of step S302, the object detected by at least one of the sensors is successfully identified as the subject H (YES in step S302), the processing proceeds to step S303.

In step S303, the control substrate 4170 causes the notification unit 4190 to notify the user that the subject H is detected as a subject state notification. For example, the notification unit 4190 notifies the user of the radiographic imaging apparatus 100 that the subject H is detected, using speaker sound, LED or other display, or communication with an external apparatus via the communication unit 4191. When notifying that the subject H is detected, the notification unit 4190 may also notify information about whether the subject H is detected via cloth and the like based on the detection states of the sensors 4121.

After the processing of step S303 in FIG. 47 is completed, the processing proceeds to step S204. In step S204, the control substrate 4170 causes the radiographic imaging apparatus 100 to transition to an imaging ready state. The processing similar to the processing of steps S205 onward described in FIG. 37 is then performed.

According to the eighteenth exemplary embodiment, whether the object detected by the sensor(s) 4121 is the subject H or an object other than the subject H is able to be identified. This can further improve the user's workability during radiographic imaging and enables quick radiographic imaging.

### Nineteenth Exemplary Embodiment

Next, a nineteenth exemplary embodiment will be described. In the following description of the nineteenth exemplary embodiment, a description of items common to the foregoing thirteenth to eighteenth exemplary embodiments is omitted, and differences from the foregoing thirteenth to eighteenth exemplary embodiments will mainly be described.

In the eighteenth exemplary embodiment, whether the detected object is the subject H or an object other than the subject H is described to be identified by using a plurality of types of sensors 4121 included in the sensor unit 4120 in combination. The nineteenth exemplary embodiment deals with a mode where a plurality of sensor units 4120 is disposed at different positions, and in which regions of the effective imaging area 4134 the subject H is disposed are determined based on detection result information from the plurality of sensor units 4120.

In the thirteenth to eighteenth exemplary embodiments described so far, the sensor unit 4120 is disposed on the connection section 4113 of the housing 4110. However, sensor units 4120 may be disposed at positions other than the connection section 4113 of the housing 4110.

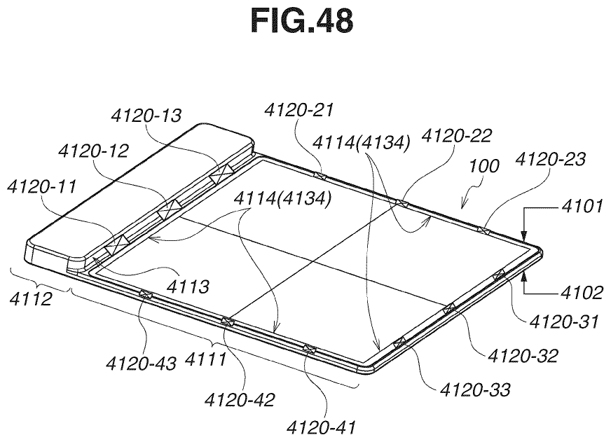

FIG. 48 is a diagram illustrating an example of a schematic configuration of the radiographic imaging apparatus 100 according to the nineteenth exemplary embodiment. In this FIG. 48, components similar to those illustrated in FIGS. 35, 36A, 36B, and 38 to 45 are denoted by the same reference numerals, and a detailed description thereof will be omitted.

As illustrated in FIG. 48, in the radiographic imaging apparatus 100 according to the nineteenth exemplary embodiment, a plurality of sensor units 4120 is disposed on the housing 4110, outside a plurality of sides of the polygonal shape (specifically, rectangular shape) that is the shape of the effective imaging area 4134.

Specifically, the radiographic imaging apparatus 100 according to the nineteenth exemplary embodiment includes a plurality of sensor units 4120-11 to 4120-13 on the connection section 4113 disposed on the housing 4110, outside a first side of the polygonal shape that is the shape of the effective imaging area 4134. The radiographic imaging apparatus 100 according to the nineteenth exemplary embodiment also includes a plurality of sensor units 4120-21 to 4120-23 on the housing 4110, outside a second side of the polygonal shape that is the shape of the effective imaging area 4134. The radiographic imaging apparatus 100 according to the nineteenth exemplary embodiment also includes a plurality of sensor units 4120-31 to 4120-33 on the housing 4110, outside a third side of the polygonal shape that is the shape of the effective imaging area 4134. The radiographic imaging apparatus 100 according to the nineteenth exemplary embodiment further includes a plurality of sensor units 4120-41 to 4120-43 on the housing 4110, outside a fourth side of the polygonal shape that is the shape of the effective imaging area 4134. To detect the position of the subject H placed on the effective imaging area 4134, the plurality of sensor units 4120-21 to 4120-23, 4120-31 to 4120-33, and 4120-41 to 4120-43 is arranged on a side with the radiation incident surface 4101 of the first thickness section (thin section) 4111. The sensor units 4120 on each side are disposed at the center position of the side and intermediate positions between the center and both ends of the side. The sensors 4121 disposed in each sensor unit 4120 may be any combination of the sensors 4121-1 to 4121-5 described in the foregoing thirteenth to seventeenth exemplary embodiment. The number and positions of sensors 4121 disposed in each sensor unit 4120 may be freely changed.

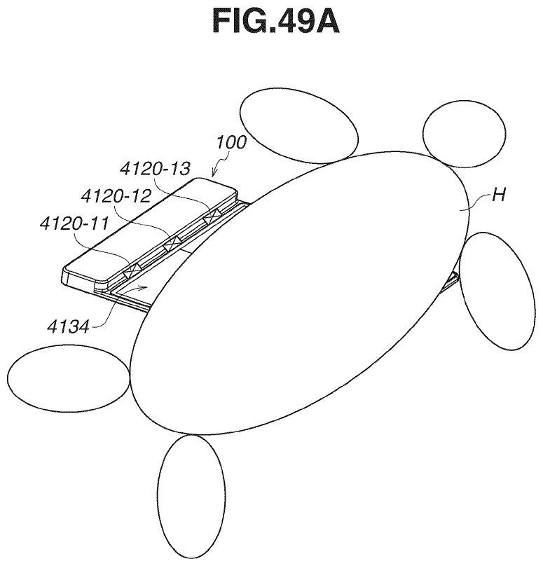

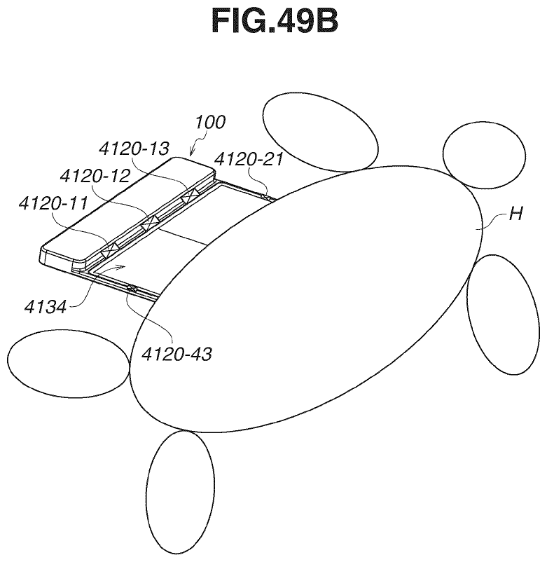

FIGS. 49A and 49B are diagrams illustrating a first example where the radiographic imaging apparatus 100 according to the nineteenth exemplary embodiment identifies the position of the subject H. In these FIGS. 49A and 49B, components similar to those illustrated in FIGS. 35, 36A, 36B, 38 to 45, and 48 are denoted by the same reference numerals, and a detailed description thereof will be omitted.

FIG. 49A illustrates an example where the subject H lies over almost the entire effective imaging area 4134. For example, chest imaging or the like of the subject H applies to this case. Here, all the sensor units 4120 illustrated in FIG. 48 detect the subject H, and the subject H is expected to be in a state capable of imaging in a desired arrangement.

Next, FIG. 49B illustrates an example where the subject H is offset toward the sensor units 4120-31 to 4120-33 illustrated in FIG. 48. In such a case, the sensor units 4120-21 and 4120-43 do not detect the subject H. If imaging is performed in the state illustrated in this FIG. 49B, there is a possibility of failure in desired imaging since the subject H is off the center of the position of the effective imaging area 4134.

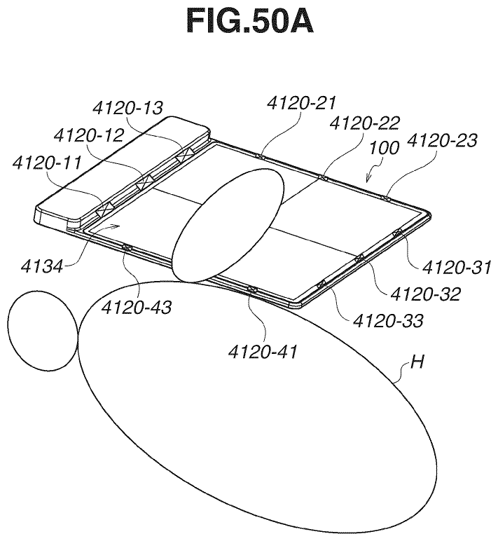

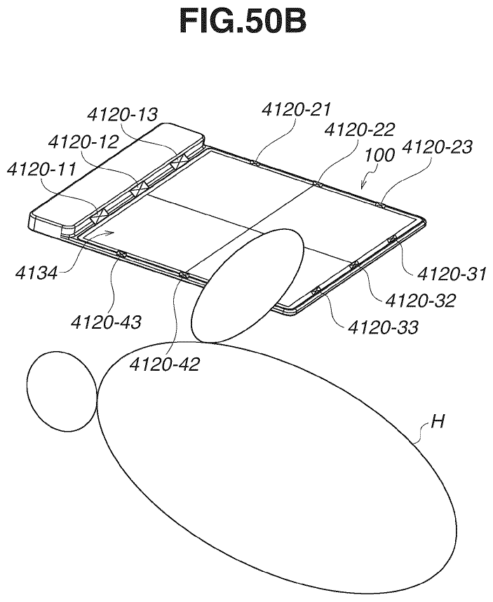

FIGS. 50A and 50B are diagrams illustrating a second example where the radiographic imaging apparatus 100 according to the nineteenth exemplary embodiment identifies the position of the subject H. In these FIGS. 50A and 50B, components similar to those illustrated in FIGS. 35, 36A, 36B, 38 to 45, and 48 are denoted by the same reference numerals, and a detailed description thereof will be omitted.

FIG. 50A illustrates an example of imaging a limb (specifically, an arm) of the subject H. In such a case, the sensor unit 4120-11 to 4120-13 and 4120-42 illustrated in FIG. 48 detect the subject H. While the subject H is detected by only some of the sensor units 4120, the subject H is expected to be in a state capable of imaging in a desired arrangement.

FIG. 50B illustrates an example of imaging a limb (specifically, an arm) of the subject H, where the subject H is offset in position. In such a case, the sensor units 4120-11, 4012-12, and 4120-41 detect the subject H. If imaging is performed in the state illustrated in this FIG. 50B, there is a possibility of failure in desired imaging since the subject H is off the center position of the effective imaging area 4134.

As described above, in the present exemplary embodiment, whether the subject H is disposed at a desired position is able to be identified based on the detection states of the plurality of sensor units 4120 disposed at different positions.

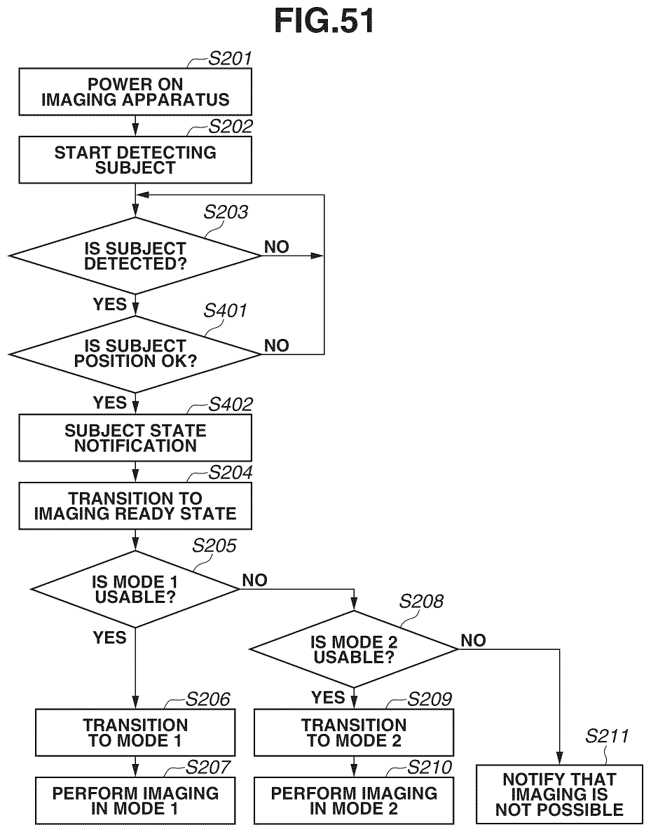

FIG. 51 is a flowchart illustrating an example of a processing procedure for a control method of the radiographic imaging apparatus 100 according to the nineteenth exemplary embodiment. In this FIG. 51, processing steps similar to those illustrated in FIG. 37 are denoted by the same step numbers, and a detailed description thereof will be omitted.

Initially, in step S201 of FIG. 51, when the radiographic imaging apparatus 100 is powered on, the control substrate 4170 supplies the power from the battery 4180 to the components of the radiographic imaging apparatus 100 and activates the radiographic imaging apparatus 100.

Next, in step S202 of FIG. 51, the control substrate 4170 starts detecting the subject H using the sensor units 4120. Specifically, in the present exemplary embodiment, the control substrate 4170 performs detection of the subject H using each of the plurality of sensors 4120-11 to 4120-13, 4120-21 to 4120-23, 4120-31 to 4120-33, and 4120-41 to 4120-43.

Next, in step S203 of FIG. 51, the control substrate 4170 determines whether the subject H is detected by any of the sensor units 4120 in the foregoing plurality of sensor units 4120-11 to 4120-43. If, as a result of this determination, the subject H is not successfully detected by any of the sensor units 4120 in the plurality of sensor units 4120-11 to 4120-43 (NO in step S203), the processing waits in step S203 until the subject H is detected by one of the sensor units 4120.

If, as a result of the determination of step S203 in FIG. 51, the subject H is detected by one of the sensor units 4120 in the plurality of sensor units 4120-11 to 4120-43 (YES in step S203), the processing proceeds to step S401.

In step S401, the control substrate 4170 determines whether the subject H is disposed at a desired position on the effective imaging area 4134 based on the detection result information from each of the sensor units 4120 (based on the detection states of the sensor units 4120 detecting the subject H). Here, it is desirable to determine the identification conditions for the position of the subject H in advance in consideration of the conditions of the subject H, the site to be imaged, etc., and store the positions and the like of the detection-needed sensor units 4120 in the storage unit 4171 of the control substrate 4170.

If, as a result of the determination of step S401, the subject H is not disposed at a desired position on the effective imaging area 4134 (NO in step S401), the processing returns to step S203. Here, the control substrate 4170 may cause the notification unit 4190 to notify the user that the subject H is not identified to be disposed at a desired position. In such a case, since the notification may last until the subject H is successfully identified to be disposed at a desired position, the notification unit 4190 desirably uses a notification method or means that does not interfere with the user's operation, such as display on the display unit.

If, as a result of the determination of step S401, the subject H is disposed at a desired position on the effective imaging area 4134 (YES in step S401), the processing proceeds to step S402.

In step S402, the control substrate 4170 causes the notification unit 4190 to notify the user that the subject H is disposed at a desired position as a subject state notification. For example, the notification unit 4190 notifies the user of the radiographic imaging apparatus 100 that the subject H is disposed at a desired position, using speaker sound, LED or other display, or communication with an external apparatus via the communication unit 4191. When notifying that the subject H is disposed at a desired position, the notification unit 4190 may also notify information about whether the subject H is detected via cloth and the like, based on the detection states of the sensors 4121 included in the sensor units 4120.

When the processing of step S402 in FIG. 51 is completed, the processing proceeds to step S204. In step S204, the control substrate 4170 causes the radiographic imaging apparatus 100 to transition to an imaging ready state. The processing of steps S205 onward described with reference to FIG. 37 is then performed.

According to the nineteenth exemplary embodiment, the subject H is able to be identified to be disposed at a desired position on the effective imaging area 4134. This can further improve the user's workability during radiographic imaging and enables quick radiographic imaging.

### Twelfth Exemplary Embodiment

Next, a twelfth exemplary embodiment will be described. In the following description of the twelfth exemplary embodiment, a description of items common to the foregoing thirteenth to nineteenth exemplary embodiments is omitted, and differences from the foregoing thirteenth to nineteenth exemplary embodiments will mainly be described.

A radiographic imaging system 10 according to the twelfth exemplary embodiment has a schematic configuration similar to that of the radiographic imaging system 10 according to the thirteenth exemplary embodiment illustrated in FIG. 35.

In the nineteenth exemplary embodiment, the detection result information from the plurality of sensor units 4120 is described to be used to identify in which region of the effective imaging area 4134 the subject H is disposed. The twelfth exemplary embodiment deals with a mode where the detection result information from sensor units 4120 is used to identify which position (region) within the effective imaging area 4134 to monitor the irradiation with radiation 201. A radiographic imaging apparatus 100 according to the twelfth exemplary embodiment is an apparatus having an auto exposure control (AEC) function. The radiographic imaging apparatus 100 according to the twelfth exemplary embodiment uses the detection result information from the sensor units 4120 in determining the position to monitor the dose (cumulative dose) of exposure to the radiation 201.

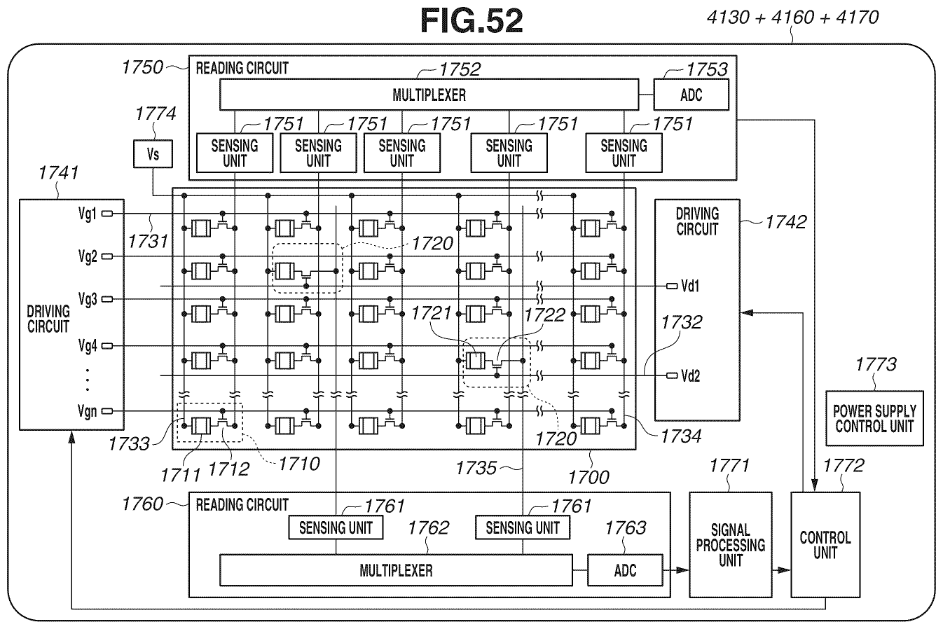

FIG. 52 is a diagram illustrating an example of a part of a schematic configuration of the radiographic imaging apparatus 100 according to the twelfth exemplary embodiment. In this FIG. 52, components similar to those illustrated in FIGS. 36A, 36B, 40, 41A, and 41B to 45 are denoted by the same reference numerals, and a detailed description thereof will be omitted. Specifically, FIG. 52 illustrates only the components included in the radiation detection panel 4130, the flexible circuit board 4160, and the control substrate 4170 in the radiographic imaging apparatus 100 according to the twelfth exemplary embodiment.

The radiation detection panel 4130 illustrated in FIG. 36A and the like includes a radiation detector 1700 and driving circuits 1741 and 1742 illustrated in FIG. 52, for example. The flexible circuit board 4160 illustrated in FIG. 36A and the like also includes reading circuits 1750 and 1760 illustrated in FIG. 52, for example. The control substrate 4170 illustrated in FIG. 36A and the like also includes a signal processing unit 1771, a control unit 1772, a power supply control unit 1773, and an element power supply circuit 1774 illustrated in FIG. 52.

The radiation detector 1700 has a function of detecting exposure to the radiation 201. The radiation detector 1700 includes a plurality of pixels arranged in a plurality of rows and a plurality of columns. In the following description, the area where the plurality of pixels is arranged in the radiation detector 1700 will be referred to as an imaging area.

The plurality of pixels disposed in the radiation detector 1700 includes a plurality of imaging pixels 1710 that converts the radiation 201 into electrical signals of a radiographic image and a plurality of sensing pixels 1720 for monitoring the irradiation with the radiation 201.

Each imaging pixel 1710 includes a first conversion element 1711 that converts the radiation 201 into an electrical signal, and a first switch element 1712 that is disposed between a column signal line 1734 and the first conversion element 1711.

Each sensing pixel 1720 includes a second conversion element 1721 that converts the radiation 201 into an electrical signal, and a second switch element 1722 that is disposed between a sensing signal line 1735 and the second conversion element 1721. The sensing pixel 1720 is arranged in the same column as some of the plurality of imaging pixels 1710.

The first conversion elements 1711 and the second conversion elements 1721 include a scintillator that converts the radiation 201 into light and photoelectric conversion elements that convert the light occurring in the scintillator into electrical signals. The scintillator is typically formed in a sheet shape to cover the imaging area, and shared by the plurality of pixels. Alternatively, the first conversion elements 1711 and the second conversion elements 1721 may be composed of conversion elements that directly convert the radiation 201 into right.

The first switch elements 1712 and the second switch elements 1722 include, for example, thin-film transistors (TFTs) having an active region formed of a semiconductor such as amorphous silicon or polycrystalline silicon (desirably, polycrystalline silicon).

The radiographic imaging apparatus 100 includes a plurality of column signal lines 1734 and a plurality of drive lines 1731. Each column signal line 1734 corresponds to one of the plurality of columns in the imaging area. Each drive line 1731 corresponds to one of the plurality of rows in the imaging area. Each drive line 1731 is driven by the driving circuit 1741.

A first electrode of the first conversion element 1711 is connected to a first main electrode of the first switch element 1712, and a second electrode of the first conversion element 1711 is connected to a bias line 1733. Here, one bias line 1733 extends in the column direction and connected in common to the second electrodes of a plurality of first conversion elements 1711 arranged in the column direction.

The bias lines 1733 receive a bias voltage Vs from the element power supply circuit 1774. The bias voltage Vs is supplied from the element power supply circuit 1774. The power supply control unit 1773 controls power supplies such as the battery 4180. The power supply control unit 1773 also controls the element power supply circuit 1774.

The second main electrodes of the first switch elements 1712 of a plurality of imaging pixels 1710 constituting a column are connected to one column signal line 1734. The control electrodes of the first switch elements 1712 of a plurality of imaging pixels 1710 constituting a row are connected to one drive line 1731. A plurality of column signal lines 1734 is connected to the reading circuit 1750. Here, the reading circuit 1750 includes a plurality of sensing units 1751, a multiplexer 1752, and an analog-to-digital converter (hereinafter, referred to as an “AD converter”) 1753.

The plurality of column signal lines 1734 is connected to respective corresponding ones of the plurality of sensing units 1751 of the reading circuit 1750. Here, one column signal line 1734 corresponds to one sensing unit 1751. The sensing units 1751 include differential amplifiers, for example. The multiplexer 1752 selects the plurality of sensing units 1751 in a predetermined order, and supplies the signal from the selected sensing unit 1751 to the AD converter 1753. The AD converter 1753 converts the supplied signal into a digital signal and outputs the digital signal.

A first electrode of the second conversion element 1721 is connected to a first main electrode of the second switch element 1722, and a second electrode of the second conversion element 1721 is connected to a bias line 1733. A second main electrode of the second switch element 1722 is connected to a sensing signal line 1735. A control electrode of the second switch element 1722 is electrically connected to a drive line 1732.

The radiographic imaging apparatus 100 includes a plurality of sensing signal lines 1735. One sensing signal line 1735 is connected with one or more sensing pixels 1720. Drive lines 1732 are driven by the driving circuit 1742. One drive line 1732 is connected with one or more sensing pixels 1720. The sensing signal lines 1735 are connected to the reading circuit 1760. Here, the reading circuit 1760 includes a plurality of sensing units 1761, a multiplexer 1762, and an AD converter 1763.

The plurality of sensing signal lines 1735 is connected to respective corresponding ones of the plurality of sensing units 1761 of the reading circuit 1760. Here, one sensing signal line 1735 corresponds to one sensing unit 1761. The sensing units 1761 include differential amplifiers, for example. The multiplexer 1762 selects the plurality of sensing units 1761 in a predetermined order, and supplies the signal from the selected sensing unit 1761 to the AD converter 1763. The AD converter 1763 converts the supplied signal into a digital signal and outputs the digital signal. The output of the reading circuit 1760 (AD converter 1763) is supplied to the signal processing unit 1771 and processed by the signal processing unit 1771. The signal processing unit 1771 outputs information indicating the irradiation of the radiographic imaging apparatus 100 with the radiation 201 based on the output of the reading circuit 1760 (AD converter 1763). Specifically, for example, the signal processing unit 1771 performs operations such as sensing the irradiation of the radiographic imaging apparatus 100 with the radiation 201 and calculating the dose (cumulative dose) of exposure to the radiation 201. When an appropriate dose (cumulative dose) of the radiation 201 is reached based on the information obtained by the signal processing unit 1771, the control unit 1772 controls the amount of irradiation of the subject H with the radiation by issuing an exposure stop notification to the radiation generation apparatus 200.

The sensing pixels 1720 may have a structure similar to that of the imaging pixels 1710. The control unit 1772 controls the driving circuit 1741, the driving circuit 1742, the reading circuit 1750, the reading circuit 1760, and the like based on information from the signal processing unit 1771, etc.

To appropriately detect the dose (cumulative dose) of exposure to the radiation 201, sensing pixels 1720 at the location where the subject H lies need to be used. In that case, the control substrate 4170 identifies in which region of the effective imaging area 4134 the subject H is disposed, and determines the sensing pixels 1720 to be used based on the identification information.

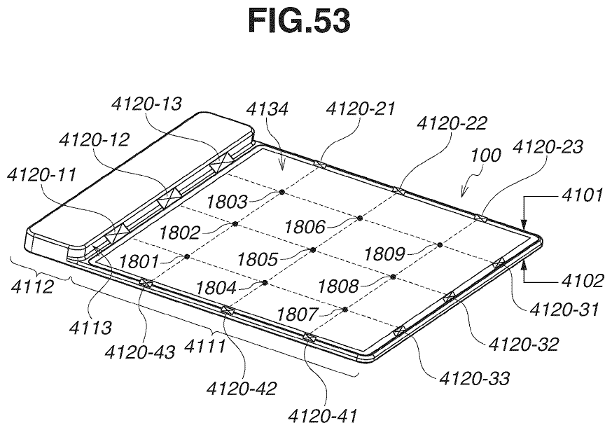

FIG. 53 is a diagram illustrating a first example of a schematic configuration of the radiographic imaging apparatus 100 according to the twentieth exemplary embodiment. In this FIG. 53, components similar to those illustrated in FIG. 48 are denoted by the same reference numerals, and a detailed description thereof will be omitted.

On the radiographic imaging apparatus 100 illustrated in FIG. 53, the intersections of line segments connecting the sensor units 4120 disposed at opposite positions among the sensor unit 4120-11 to 4120-43 are set as subject detection points 1801 to 1809. The control substrate 4170 then selects and uses the sensing pixels 1720 disposed at the subject detection points 1801 to 1809 depending on the detection states of the sensor units 4120.

For example, as illustrated in FIG. 49A, if the subject H lies over almost the entire effective imaging area 4134, the dose of the radiation 201 is able to be appropriately detected at the subject detection points 1801 to 1809. It will be understood that all the sensing pixels 1720 disposed at the subject detection points 1801 to 1809 may be selected and used, or any one or ones of the sensing pixels 1720 may be selected and used.

For example, in the case illustrated in FIG. 49B, the subject H is offset toward the sensor units 4120-31 to 4120-33, and the sensor units 4120-21 and 4120-43 do not detect the subject H. In such a case, the sensing pixels 1720 disposed at the subject detection points 1801 to 1803 are not used, and the sensing pixels 1702 disposed at the subject detection points 1804 to 1809 are used.

For example, in the case illustrated in FIG. 50A, the subject H is detected by the sensor units 4120-11 to 4120-13 and 4120-42. The sensing pixel 1720 disposed at the subject detection point 1804 is thus used.

For example, in the case illustrated in FIG. 50B, the subject H is detected by the sensor units 4120-11, 4120-12, and 4120-41. The sensing pixel 1720 disposed at the subject detection point 1807 is thus used.

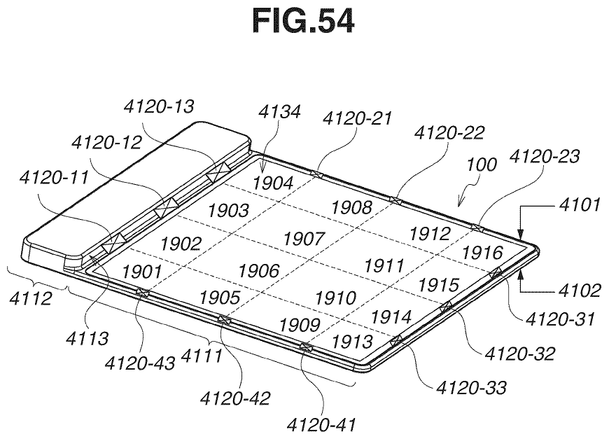

FIG. 54 is a diagram illustrating a second example of the schematic configuration of the radiographic imaging apparatus 100 according to the twentieth exemplary embodiment. In this FIG. 54, components similar to those illustrated in FIGS. 48 and 53 are denoted by the same reference numerals, and a detailed description thereof will be omitted.

In the radiographic imaging apparatus 100 illustrated in FIG. 54, the effective imaging area 4134 is divided by line segments connecting the sensor units 4120 disposed at opposite positions among the sensor units 4120-11 to 4120-43, and the divided areas are set as subject detection areas 1901 to 1916. The control substrate 4170 then selects and uses the sensing pixels 1720 disposed in the subject detection areas 1901 to 1916 depending on the detection states of the sensor units 4120, in the similar manner as described with reference to FIG. 53.

According to the twentieth exemplary embodiment, the sensing pixels 1720 to be used in monitoring the irradiation with the radiation 201 are set based on the detection result information from the sensor units 4120, and the user's workability during radiographic imaging is thus further improved. This enables quick radiographic imaging.

The foregoing thirteenth to twentieth exemplary embodiments of the present invention are merely examples of specific implementations for carrying out the present invention, and the technical scope of the present invention should not be interpreted as being limited thereto. In other words, the present invention can be practiced in various forms without departing from the technical concept or essential features thereof.

The thirteenth to twentieth exemplary embodiments of the present invention include the following configurations.

### [Configuration 50]

A radiographic imaging apparatus comprising:
a radiation detection panel configured to include an effective imaging area where radiation transmitted through a subject is detected;
a housing configured to accommodate the radiation detection panel, the housing including the effective imaging area having a polygonal shape as seen from a side where the radiation is incident; and
a sensor unit configured to include one or more types of sensors for detecting the subject, the sensor unit being disposed on the housing at a position outside at least one side of the polygonal shape of the effective imaging area.

### [Configuration 51]

The radiographic imaging apparatus according to Configuration 50, wherein the sensor unit is disposed on a position close to a side of the housing where the radiation is incident.

### [Configuration 52]

The radiographic imaging apparatus according to Configuration 50 or 51, wherein the housing includes
a first thickness section including the effective imaging area and having a first thickness,
a second thickness section not including the effective imaging area and having a second thickness different from the first thickness, and
a connection section connecting the first thickness section and the second thickness section.

### [Configuration 53]

The radiographic imaging apparatus according to Configuration 52, wherein the second thickness section is thicker than the first thickness section toward the side where the radiation is incident.

### [Configuration 54]

The radiographic imaging apparatus according to Configuration 52 or 53,
wherein the connection section connects the first thickness section and the second thickness section along a perpendicular or an oblique line, and
wherein the sensor unit is disposed on the connection section.

### [Configuration 55]

The radiographic imaging apparatus according to any one of Configurations 50 to 54, further including a control unit configured to detect the subject based on detection result information from the sensor unit, and if the subject is detected, cause the radiographic imaging apparatus to transition to an imaging ready state.

### [Configuration 56]

The radiographic imaging apparatus according to Configuration 55, further including a storage unit configured to store information indicating use order of a plurality of imaging modes,
wherein the control unit is configured to, when causing the radiographic imaging apparatus to transition to the imaging ready state, cause the radiographic imaging apparatus to transition to an imaging mode of highest order among the plurality of imaging modes based on the information indicating the use order.

### [Configuration 57]

The radiographic imaging apparatus according to Configuration 55 or 56, wherein the control unit is configured to identify whether a detected object is the subject or an object other than the subject, based on detection result information from the sensor unit, and if the detected object is the subject, cause the radiographic imaging apparatus to transition to the imaging ready state.

### [Configuration 58]

The radiographic imaging apparatus according to any one of Configurations 55 to 57,
wherein a plurality of sensor units is disposed at different positions, and
wherein the control unit is configured to detect a position of the subject on the effective imaging area, based on detection result information from the plurality of sensor units, and cause the radiographic imaging apparatus to transition to the imaging ready state based on the detected position of the subject.

### [Configuration 59]

The radiographic imaging apparatus according to Configuration 58, wherein the plurality of sensor units is disposed, on the housing, outside a plurality of sides of the polygonal shape of the effective imaging area.

### [Configuration 60]

The radiographic imaging apparatus according to any one of Configurations 55 to 59,
wherein the radiation detection panel includes a plurality of imaging pixels and a plurality of sensing pixels within the effective imaging area, the plurality of imaging pixels being configured to convert the radiation into electrical signals of a radiographic image, the plurality of sensing pixels being configured to monitor irradiation with the radiation.

### [Configuration 61]

The radiographic imaging apparatus according to any one of Configurations 55 to 60, further including a notification unit configured to issue notification of a detection state of the subject by the control unit.

### [Configuration 62]

The radiographic imaging apparatus according to Configuration 61, wherein the notification unit is configured to, if a variation exceeding a predetermined level occurs in the subject, issue the notification.

### [Configuration 63]

The radiographic imaging apparatus according to Configuration 61 or 62, wherein the notification unit is configured to issue the notification using sound, display, or communication via a wired communication unit or a wireless communication unit.

### [Configuration 64]

The radiographic imaging apparatus according to any one of Configurations 50 to 63, wherein the sensor is an infrared sensor.

### [Configuration 65]

The radiographic imaging apparatus according to any one of Configurations 50 to 63, wherein the sensor is an ultrasonic sensor.

### [Configuration 66]

The radiographic imaging apparatus according to any one of Configurations 50 to 63, wherein the sensor is a capacitive sensor.

### [Configuration 67]

The radiographic imaging apparatus according to any one of Configurations 50 to 63, wherein the sensor is a magnetic sensor.

### [Configuration 68]

The radiographic imaging apparatus according to any one of Configurations 50 to 63, wherein the sensor is a proximity wireless sensor.

### [Configuration 69]

A radiographic imaging system including:
the radiographic imaging apparatus according to any one of Configurations 50 to 68; and
a radiation generation apparatus configured to generate the radiation toward the subject.

According to the features set forth in Configurations 50 to 69, the user's workability during radiographic imaging is improved, and quick radiographic imaging is enabled.

### Twenty-First Exemplary Embodiment

Next, a twenty-first exemplary embodiment will be described.

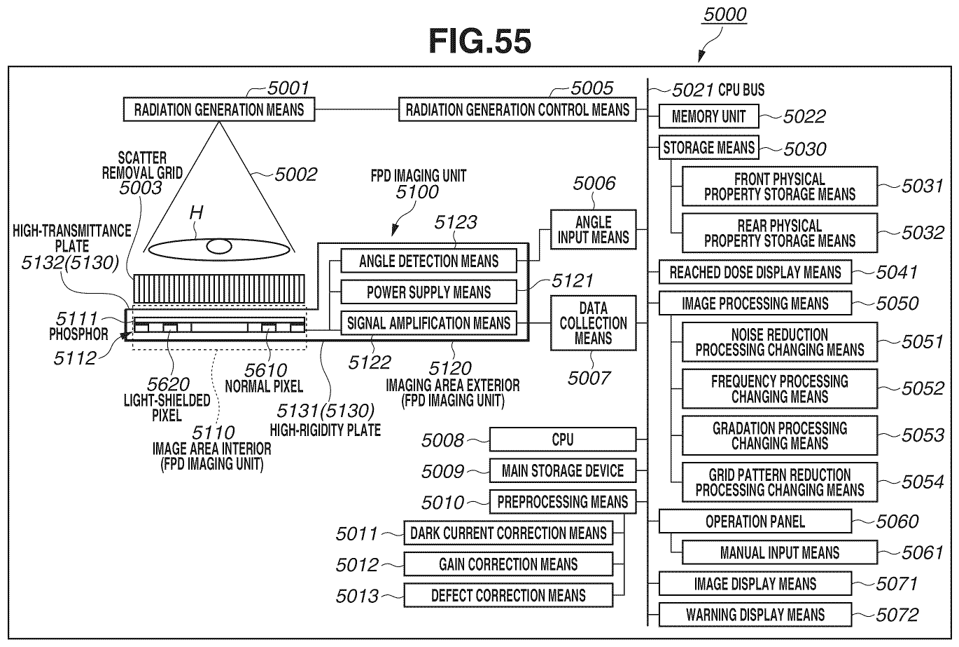

FIG. 55 is a diagram illustrating an example of a schematic configuration of a radiographic imaging apparatus 5000 according to the twenty-first exemplary embodiment. The radiographic imaging apparatus 5000 illustrated in FIG. 55 may be used for medical purposes in particular.

The radiographic imaging apparatus 5000 illustrated in FIG. 55 includes a radiation generation means 5001, a scatter removal grid 5003, an FPD imaging unit 5100, a radiation generation control means 5005, an angle input means 5006, a data collection means 5007, a CPU 5008, and a main storage device 5009. The radiographic imaging apparatus 5000 also includes a preprocessing means 5010, a CPU bus 5021, a memory unit 5022, a storage means 5030, a reached dose display means 5041, an image processing means 5050, an operation panel 5060, an image display means 5071, and a warning display means 5072.

The radiation generation means 5001 emits radiation 5002 toward a subject H and the FPD imaging unit 5100 based on control of the radiation generation control means 5005.

The FPD imaging unit 5100 is a component unit that detects incident radiation 5002 and captures a radiographic image. A housing 5130 of the FPD imaging unit 5100 and its interior are divided into an imaging area interior 5110 that is within the range of an imaging area to be irradiated with the radiation 5002 and an imaging area exterior 5120 that is outside the range of the imaging area. The imaging area interior 5110 includes a phosphor 5111 that converts the incident radiation 5002 into light, and a pixel array 5112 where a plurality of pixels including photoelectric conversion elements for converting the light occurring in the phosphor 5111 into electrical signals of a radiographic image is arranged. The pixel array 5112 illustrated in FIG. 55 includes a plurality of normal pixels 5610 and a plurality of light-shielded pixels 5620. The imaging area exterior 5120 includes a printed board (not illustrated) equipped with electronic parts (electronic parts attached to an insulating board), a power supply means 5121, a signal amplification means 5122, and an angle detection means 5123. In the present exemplary embodiment, examples of the electronic parts mounted on the printed board (not illustrated) include electronic parts that perform signal communication with the pixel array 5112 and electronic parts that supply power to the pixel array 5112. Examples of the electronic parts that perform signal communication with the pixel array 5112 include electronic parts that transmit driving control signals to the pixel array 5112 and electronic parts that receives the electrical signals of a radiographic image from the pixel array 5112. The housing 5130 of the FPD imaging unit 5100 accommodates the phosphor 5111, the pixel array 5112, the printed board (not illustrated), the power supply means 5121, the signal amplification means 5122, the angle detection means 5123, etc.

The preprocessing means 5010 includes a dark current correction means 5011, a gain correction means 5012, and a defect correction means 5013. The storage means 5030 includes a front physical property storage means 5031 for situations where the radiation 5002 is incident on the front surface of the housing 5130 of the FPD imaging unit 5100, and a rear physical property storage means 5032 for situations where the radiation 5002 is incident on the rear surface of the housing 5130. The image processing means 5050 includes a noise reduction processing changing means 5051, a frequency processing changing means 5052, a gradation processing changing means 5053, and a grid pattern reduction processing changing means 5054. The operation panel 5060 includes a manual input means 5061.

When an imaging order arrives, the user who is a medical practitioner sets imaging conditions via the operation panel 5060. The imaging order includes information about the imaging site, physique, age, purpose of imaging, etc. The imaging conditions to be set include the tube voltage and tube current of the radiation generation means 5001, the irradiation time of radiation 5002, the type of scatter removal grid 5003, and the body position of the patient who is the subject H. The imaging conditions are set from an information apparatus including the CPU 5008 and the main storage device 5009 into the radiation generation means 5001 and the FPD imaging unit 5100 equipped with a two-dimensional flat radiation detection means including the phosphor 5111 and the pixel array 5112 via the CPU bus 5021.

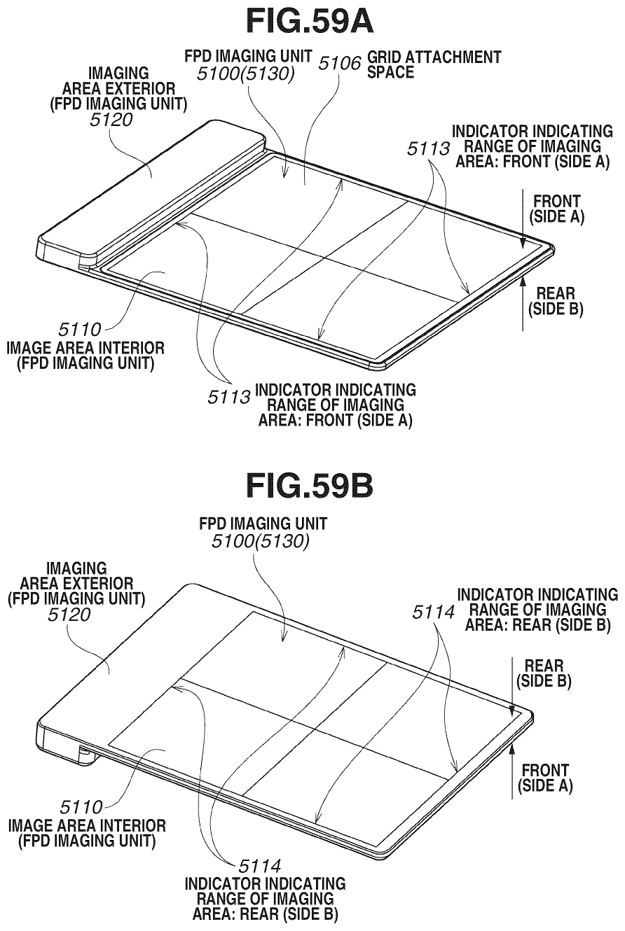

In the present exemplary embodiment, based on requests included in the foregoing imaging order and imaging conditions, a recommended imaging direction (front or rear of the FPD imaging unit 5100) is displayed on a screen of the image display means 5071 or a screen of the operation panel 5060. Based on the information about the appropriate incident direction of the radiation 5002 displayed on the screen, the user positions the patient (subject) and the FPD imaging unit 5100. The housing 5130 of the FPD imaging unit 5100 displays indicators indicating the range of the imaging area (indicators 5113 and 5114 of FIGS. 59A and 59B to be described below) in two directions (may be more than two directions), on the front surface and the rear surface. In the example illustrated in FIG. 55, the housing 5130 of the FPD imaging unit 5100 includes a high-rigidity plate 5131 and a high-transmittance plate 5132.

The user positions the patient (examinee) who is the subject H and the FPD imaging unit 5100. Moreover, the user narrows the irradiation range of the radiation 5002 from the radiation generation means 5001 so that the irradiation range of the radiation 5002 does not significantly exceed the range of the imaging area displayed in two directions on the front and rear surfaces of the housing 5130 to prevent irradiation with unnecessary exposure doses.

When positioning the FPD imaging unit 5100, the user can figure out which side of the housing 5130 of the FPD imaging unit 5100, the front surface or the rear surface, is facing the radiation generation means 5001. The user therefore desirably inputs the incident direction of the radiation 5002 from the manual input means 5061 before imaging.

As described above, the radiation generation means 5001 emits the radiation 5002 toward the subject H such as a human body. The FPD imaging unit 5100 is a FPD (Flat Panel Detector) that includes the two-dimensional flat radiation detection means including the phosphor 5111 and the pixel array 5112, and generates radiographic image data and offset signals. In the present exemplary embodiment, imaging is able to be performed in two directions: with the radiation 5002 incident on the imaging area interior 5110 from the side with the phosphor 5111 and with the radiation 5002 incident from the side with the pixel array 5112. The pixel array 5112 of the foregoing two-dimensional flat radiation detection means includes a large number of pixels arranged on a large planar wafer, with the normal pixels 5610 and the light-shielded pixels 5620 included in the effective pixel area.

The imaging area exterior 5120 of the FPD imaging unit 5100 includes a lot of electrical parts such as the printed board mentioned above (not illustrated). The imaging area interior 5110 does not include many of the electrical parts, and is thus configured as a thin section. Regarding the material of the housing 5130 of the FPD imaging unit 5100, materials with high transmittance for the radiation 5002 generally tend to have low rigidity. Either the front surface or the rear surface of the housing 5130 of the FPD imaging unit 5100 is therefore desirably formed of a material with high transmittance for the radiation 5002 (high-radiation-transmittance material), and the other a material with high rigidity (high-rigidity material). The housing 5130 of the FPD imaging unit 5100 illustrated in FIG. 55 is configured such that the front portion close to the phosphor 5111 is composed of the high-transmittance plate 5132 formed of high-rigidity material, and the rear portion close to the pixel array 5112 is composed of the high-rigidity plate 5131 formed of high-transmittance material. The purpose is to allow a large amount of radiation 5002 to pass through the phosphor 5111 accommodated in the housing 5130 of the FPD imaging unit 5100 and to securely protect the pixel array 5112, the phosphor 5111, and the like from external force.

The radiation 5002 incident on the imaging area interior 5110 of the FPD imaging unit 5100 is converted into light (visible light) by the phosphor 5111. In FIG. 55, the phosphor 5111 is disposed on only one side (upper side) of the pixel array 5112. However, in the present exemplary embodiment, the phosphor 5111 may be disposed on both sides (upper and lower sides). If the phosphor 5111 is disposed on both sides (upper and lower sides) as seen from the pixel array 5112, the phosphor 5111 that converts more radiation 5002 into visible light can be considered to be illustrated in FIG. 55.

The photoelectric conversion elements in the normal pixels 5610 photoelectrically convert the visible light emitted from the phosphor 5111 into electrical signals of a radiographic image. Meanwhile, the light-shielded pixels 5620 are shielded from light by a metal or other light-shielding mask between the phosphor 5111 and their photoelectric conversion elements and even up to a part of adjoining pixels, so that photoelectric conversion will not occur when the radiation 5002 or visible light is incident thereon.

Immediately after radiographic imaging, the electrical signals of the radiographic image obtained by the photoelectric conversion elements are driven and read by a gate driving circuit and a reading circuit, amplified by the signal amplification means 5122, and then converted from analog signals into digital signals (radiographic image signals). The radiographic image signals are then transmitted from the FPD imaging unit 5100 to the data collection means 5007. The radiographic image signals (which form a radiographic image when rearranged) obtained by the data collection means 5007 are subjected to preprocessing in the preprocessing means 5010, and then display image processing and the like in the image processing means 5050. The image-processed radiographic image is finally displayed on the image display means 5071 as a diagnostic image. The radiographic image is used not only as a diagnostic image but in detecting the incident direction of the radiation 5002 as well. For example, the angle detection means 5123 detects the incident angle of the radiation 5002 with respect to the FPD imaging unit 5100 by statistically analyzing differences in the pixel outputs (pixel values) of the normal pixels 5610 and the light-shielded pixels 5620, and as a result, detects the incident direction of the radiation 5002. For example, assuming the range of the incident angle of the radiation 5002 with respect to the FPD imaging unit 5100 as 0° to 360°, the incident direction of the radiation 5002 is detected as being the front if the incident angle is greater than or equal to 0° and less than 180° (other numerical values may be employed). Moreover, if, for example, the incident angle is greater than or equal to 180° and less than 360° (other numerical values may be employed), the incident direction of the radiation 5002 is detected as being the rear.

The angle detection means 5123 also detects the incident angle of the radiation 5002 input from the angle input means 5006, which is one of automatic input means, or the manual input means 5061, and as a result, detect the incident direction of the radiation 5002. Specifically, the angle detection means 5123 detects whether the incident angle of the radiation 5002 with respect to the imaging area interior 5110 is a first incident direction from a side with the phosphor 5111 (front side) or a second incident direction from a side with the pixel array 5112 (rear side). Here, the first incident direction and the second incident direction are opposite directions.

The radiographic image transmitted to the preprocessing means 5010 is passed through the dark current correction means 5011, the gain correction means 5012, and the defect correction means 5013 of the preprocessing means 5010, and subjected to QA processing in the image processing means 5050. The radiographic imaging apparatus 5000 according to the present exemplary embodiment desirably stores physical property values specific to the model of radiographic imaging apparatus in the front physical property storage means 5031 and the rear physical property storage means 5032 of the storage means 5030 before shipment. As employed herein, physical property values refer to image quality characteristic values of radiographic images. More specifically, the front physical property storage means 5031 stores the image quality characteristic values of radiographic images obtained based on the radiation incident from the side with the foregoing phosphor 5111 (front side) in the first incident direction. The rear physical property storage means 5032 stores the image quality characteristic values of radiographic images obtained based on the radiation incident from the side with the foregoing pixel array 5112 (rear side) in the second incident direction. For example, as the physical property values (image quality characteristic values), the physical property storage means 5031 and 5032 store at least one value among pixel values dependent on radiation dose, noise values dependent on radiation dose, and sharpness values dependent of the frequency of a radiographic image.

The image processing means 5050 performs different image processing between a first radiographic image based on the radiation 5002 incident on the imaging area interior 5110 from the side with the phosphor 5111 and a second radiographic image based on the radiation 5002 incident from the side with the pixel array 5112. The image processing means 5050 performs the image processing based on the detection result of the angle detection means 5123 (first incident direction or second incident direction). Here, the image processing means 5050 selects the physical property values (image quality characteristic values) from the front physical property storage means 5031 or the rear physical property storage means 5032 based on the detection result of the angle detection means 5123, and performs image processing based on the selected physical property values (image quality characteristic values).

As the QA processing, the image processing means 5050 performs different image processing between the foregoing first radiographic image and the second radiographic image by changing image processing parameters. The noise reduction processing changing means 5051 of the image processing means 5050 is a first changing means for changing noise reduction processing parameters for a radiographic image. The frequency processing changing means 5052 of the image processing means 5050 is a second changing means for changing frequency processing parameters for a radiographic image. The gradation processing changing means 5053 of the image processing means 5050 is a third changing means for changing gradation processing parameters for a radiographic image. The grid pattern reduction processing changing means 5054 of the image processing means 5050 is a fourth changing means for changing grid pattern reduction processing parameters for a radiographic image. In the present exemplary embodiment, it is sufficient for the image processing means 5050 to include at least one of the noise reduction processing changing means 5051, the frequency processing changing means 5052, the gradation processing changing means 5053, and the grid pattern reduction processing changing means 5054.

The radiographic imaging apparatus 5000 also includes the reached dose display means 5041. For example, the reached dose display means 5041 displays an EI value (Exposure Index value) as the reached dose. A table for converting pixel values into EI values in calculating the EI value from the pixel values of the respective pixels in the pixel array 5112 is based on the physical property values (image quality characteristic values). In the present exemplary embodiment, the pixel values are converted into different EI values depending on whether the incident direction of the radiation 5002 is the front (phosphor side) or the rear (photoelectric element side) of the housing 5130. For that purpose, the reached dose display means 5041 selects appropriate physical property values (image quality characteristic values) from the front physical property storage means 5031 and the rear physical property storage means 5032 depending on the incident direction of the radiation 5002, and calculates and displays the reached dose. Note that the reached dose display means 5041 may be implemented as an FPGA inside the FPD imaging unit 5100.

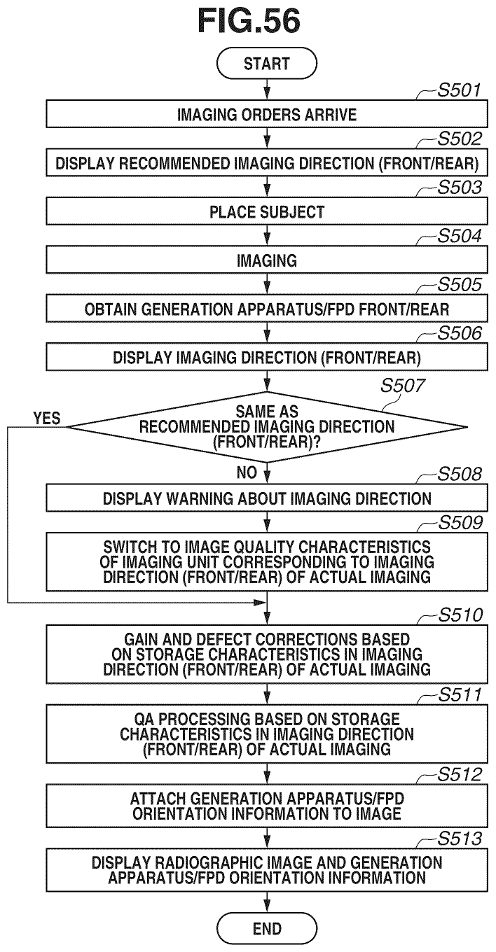

FIG. 56 is a flowchart illustrating an example of a processing procedure from a start to an end of radiographic imaging of the subject H using the radiographic imaging apparatus 5000 illustrated in FIG. 55.

Initially, in step S501 of FIG. 56, imaging orders from a medical practitioner such as a doctor arrive at the imaging department in advance of the imaging of the subject H. These imaging orders include the imaging site, physique, age, and purpose of imaging.

Next, in step S502, the radiographic imaging apparatus 5000 displays whether the recommended imaging direction is the front (phosphor side) or the rear (pixel array side) on the operation panel 5060 or the image display means 5071 based on the foregoing imaging orders (and physical property values). The operation panel 5060 or the image display means 5071 performing the processing of this step S502 corresponds to a direction display means that displays the recommended imaging direction (recommended incident direction of the radiation 5002). For example, when the imaging age in the imaging orders is that of a pediatric subject, the front (side A/blue side) is displayed for reduced exposure dose if the incident direction of radiation for high sensitivity, or high DQE (Detective Quantum Efficiency), is the front (phosphor side). For example, when the main purpose of imaging is to detect fractures, the rear (side B/green side) is displayed if the incident direction of radiation for high sharpness, or high MTF (Modular Transfer Function), is the rear (pixel array side). If the imaging orders include follow-up observation or temporal change, the same side of the housing 5130 as in the previous imaging may be displayed as the recommended side.

Next, in step S503, the medical practitioner (user) places the subject H.

The subject H is placed between the FPD imaging unit 5100 and the radiation generation means 5001 and as close to the FPD imaging unit 5100 as possible. The FPD imaging unit 5100 of the present exemplary embodiment is capable of radiographic imaging with the radiation 5002 incident from either the front surface or the rear surface of the housing 5130. Here, the subject H is placed in the direction recommended in step S502. If the subject H is large in thickness, the placement of the subject H in step S503 includes installing the scatter removal grid 5003 and the like.

Next, in step S504, the radiographic imaging apparatus 5000 causes the radiation generation means 5001 to generate radiation 5002, and causes the FPD imaging unit 5100 to capture a radiographic image of the subject H.

Next, in step S505, the radiographic imaging apparatus 5000 (angle detection means 5123) detects from which direction the radiation 5002 is incident, the front or rear of the housing 5130 of the FPD imaging unit 5100, during the imaging in step S504. For example, the angle detection means 5123 detects the incident direction of the radiation 5002 based on information input from the manual input means 5061 or from the automatic input means that uses the light-shielded pixels 5620, an acceleration measurement element including a piezoelectric element, or markers disposed in the imaging area interior 5110.

Next, in step S506, the radiographic imaging apparatus 5000 displays the imaging direction (front or rear) that is the incident direction of the radiation 5002 on the image display means 5071 or the operation panel 5060.

Next, in step S507, the radiographic imaging apparatus 5000 determines whether the actual imaging direction (front or rear) displayed in step S506 coincides with the recommended imaging direction (front or rear) displayed in step S502.

If, as a result of the determination of step S507, the actual imaging direction (front or rear) displayed in step S506 does not coincide with the recommended imaging direction (front or rear) displayed in step S502 (NO in step S507), the processing proceeds to step S508.

In step S508, the radiographic imaging apparatus 5000 displays a warning on the warning display means 5072 that the actual imaging direction is not the recommended imaging direction. Possible reasons why the imaging directions do not coincide with each other include that the medical practitioner makes a mistake because the front and rear of the FPD imaging unit 5100 are difficult to see due to infection control measures, and that immediacy has priority over image quality due to factors such as restrictions in the posture of the subject H and timing. In the radiographic imaging apparatus 5000 according to the present exemplary embodiment, the need for reimaging is reduced by the processing of the image processing means 5050 even if the medical practitioner mistakes the front and rear of the FPD imaging unit 5100.

Next, in step S509, the radiographic imaging apparatus 5000 switches between the physical property values (image quality characteristic values) of the front physical property storage means 5031 and those of the rear physical property storage means 5032 based on the imaging direction (front or rear) of the actual imaging. Here, the physical property values (image quality characteristic values) may include the reached dose of the radiation based on the pixel values.

Next, in step S510, the radiographic imaging apparatus 5000 performs gain correction and the like on the captured radiographic image using the preprocessing means 5010 based on storage characteristics in the imaging direction (front or rear) of the actual imaging.

Next, in step S511, the radiographic imaging apparatus 5000 performs noise reduction processing, frequency processing, gradation processing, and the like using the image processing means 5050 based on the physical property values (image quality characteristic values) set in step S509. For example, the physical property values (image quality characteristic values) set in step S509 include pre-shipment machine learning values for noise reduction processing using deep learning.

Next, in step S512, the radiographic imaging apparatus 5000 adds generation apparatus/FPD orientation information indicating the imaging direction (front or rear) to the header of the radiographic image obtained by the imaging, as well as the model of imaging apparatus and a serial number. The dose index value (EI value) is also appropriately output using the physical characteristic values (image quality characteristic values) corresponding to the incident direction of the radiation 5002, and attached to the radiographic image.

Next, in step S513, the radiographic imaging apparatus 5000 displays the radiographic image and generation apparatus/FPD orientation information obtained by the imaging on the image display means 5071 as needed. The medical practitioner checks the radiographic image and the like displayed on the image display means 5071, and if there are no issues, the imaging is completed. The processing of the flowchart illustrated in FIG. 56 ends thus.

FIGS. 57A-1, 57A-2, 57B-1, and 57B-2 are diagrams for describing the principle behind differences in the image quality characteristics when radiographic images are captured with the radiation 5002 incident from the front and the rear of the housing 5130 of the FPD imaging unit 5100 illustrated in FIG. 55. In these FIGS. 57A-1, 57A-2, 57B-1, and 57B-2, components similar to those illustrated in FIG. 55 are denoted by the same reference numerals, and a detailed description thereof will be omitted.

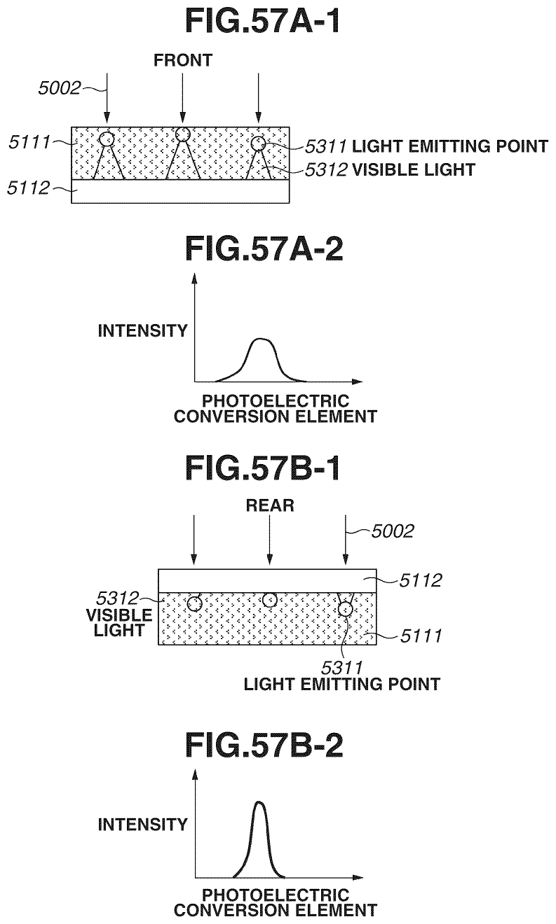

For the sake of convenience, in the present exemplary embodiment, the incident direction of the radiation 5002 defines the side with the phosphor 5111 as illustrated in FIG. 57A-1 as the front, and the incident direction of the radiation 5002 defines the side with the pixel array 5112 as illustrated in FIG. 57B-1 as the rear. The front and rear may be replaced with terms easily understandable to medical practitioners, such as side A and side B, direction 1 and direction 2, and a blue side and a green side.

In the case illustrated in FIG. 57A-1 where the incident direction of the radiation 5002 is the front of the FPD imaging unit 5100, the radiation 5002 incident on the FPD imaging unit 5100 is converted into visible light 5312 in the phosphor 5111. As a physical phenomenon, light emitting points 5311 tend to emit light near the incident side. When the incident direction of the radiation 5002 is the front of the FPD imaging unit 5100, the visible light 5321 therefore reaches the pixel array 5112 over some distance. As a result, the visible light 5321 spreads out before reaching the pixel array 5112, and the radiographic image has a low sharpness (MTF) as illustrated in FIG. 57A-2.

On the other hand, if the incident direction of the radiation 5002 illustrated in FIG. 57B-1 is the rear of the FPD imaging unit 5100, the light emitting points 5311 are disposed near the pixel array 5112. This can relatively reduce the spreading of the visible light 5312, and the sharpness (MTF) of the radiographic image becomes relatively high as illustrated in FIG. 57B-2. Moreover, since the radiation 5002 passes through the pixel array 5112 before reaching the phosphor 5111, the sensitivity (DQE) becomes slightly lower.

As illustrated in these FIGS. 57A-1, 57A-2, 57B-1, and 57B-2, even when the same FPD imaging unit 5100 is used, the physical property values (image quality characteristic values) of the radiographic image vary depending on whether the incident direction of the radiation 5002 is the front or rear. The image processing means 5050 therefore changes the image processing therebetween. The image processing desirably includes not only gradation processing and the like for matching visual appearance but also changing the grid pattern reduction processing specific to the radiographic imaging apparatus 5000. The reason is that if the sharpness of the grid pattern in the radiographic image varies depending on whether the radiation 5002 is incident on the front or the rear, the grid pattern may remain due to insufficient image processing. For example, the EI value calculated from the pixel values of the FPD imaging unit 5100 is required to be output and displayed as a dose index value. In calculating the dose index value, the physical property values (image quality characteristic values) are also changed based on the radiation incident direction since the physical property values (image quality characteristic values) of the radiographic image differ between front incidence and rear incidence. Note that while, in FIGS. 57A-1 and 57B-1, the phosphor 5111 is disposed on only one side of the pixel array 5112, the phosphor 5111 may be disposed on both sides. If the phosphor 5111 is disposed on both sides of the pixel array 5112, FIGS. 57A-1 and 57B-1 may be interpreted as illustrating the phosphor 5111 on the side where more radiation 5002 is converted into visible light 5312.

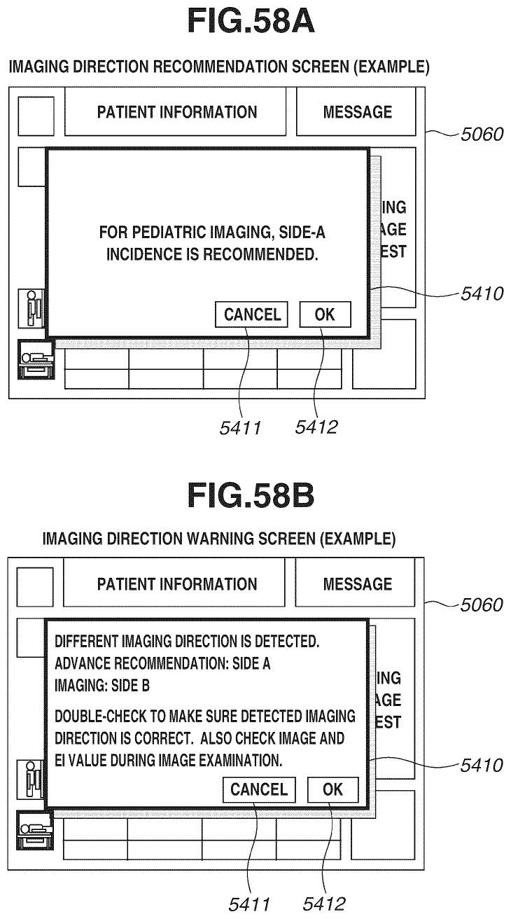

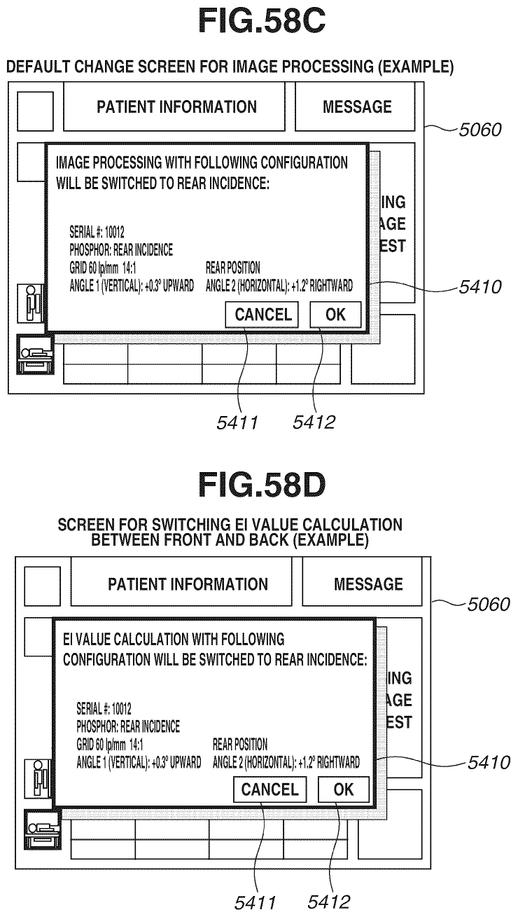

FIGS. 58A to 58D are diagrams illustrating examples of an operation screen displayed on the operation panel 5060 illustrated in FIG. 55. This operation screen includes a display area 5410, and a cancel button 5411 and an OK button 5412 in the display area 5410.

FIG. 58A illustrates an example of a screen for recommending the imaging direction before imaging, based on imaging orders including the imaging site, physique, age, and purpose of imaging. For pediatric imaging, the recommendation for the imaging direction that provides high sensitivity is displayed in advance. For limb imaging, the recommendation for the imaging direction that provides high resolution is displayed in advance.

FIG. 58B illustrates an example of an imaging direction warning screen for situations where the imaging direction recommended in advance and the input/detected imaging direction are different. Along with the display of the imaging directions, the screen also prompts a check since the input/detected imaging direction may be an erroneously input or erroneously detected one. The screen also desirably prompts a check since the displayed image, dose index value, or EI value may be based on wrong physical property values (image quality characteristic values).

FIG. 58C illustrates an example of a default change screen for the image processing. This screen is intended to prompt a change when the imaging direction recommended in advance and the input/detected imaging direction are different, since the image processing may be based on wrong physical property values (image quality characteristic values).

FIG. 58D illustrates an example of a screen for switching EI value calculation between the front and rear. This screen is intended to prompt a change when the imaging direction recommended in advance and the input/detected imaging direction are different, since the dose index value such as the EI value may be based on wrong physical property values (image quality characteristic values).

While FIGS. 58A to 58D illustrate the operation screens displayed on the operation panel 5060, the screens may be those of the image display means 5071 or those of the dedicated warning display means 5072. While FIGS. 58A to 58D illustrate examples of the screens before image examination immediately after imaging, the examples may be those of screens during secondary image examination or diagnosis.

FIGS. 59A and 59B are diagrams illustrating an example of the appearance of the FPD imaging unit 5100 illustrated in FIG. 55.

The FPD imaging unit 5100 is divided into two regions: the imaging area interior 5110 where the phosphor 5111, the pixel array 5112, and the like are disposed, and the imaging area exterior 5120 where the printed board and the like are disposed. Specifically, FIG. 59A is a view of the FPD imaging unit 5100 from the front (side A). FIG. 59B is a view of the FPD imaging unit 5100 from the rear (side B).

The thickness of the imaging area interior 5110 is reduced since the printed board, the power supply means 5121 such as a battery, the signal amplification means 5122 such as an amplifier IC, the angle detection means 5123, and the like are not included. Compared to the imaging area interior 5110, the imaging area exterior 5120 is a thick section since the printed board, the power supply means 5121, the signal amplification means 5122, the angle detection means 5123, and the like are included. In other words, the imaging area interior 5110 and the imaging area exterior 5120 of the housing 5130 of the FPD imaging unit 5100 have different thicknesses, with the imaging area interior 5110 thinner than the imaging area exterior 5120. A grid mounting space 5160 is desirably provided by utilizing the space where the imaging area interior 5110 and the imaging area exterior 5120 differ in thickness.

The housing 5130 of the FPD imaging unit 5100 illustrated in FIGS. 59A and 59B displays indicators 5113 and 5114 indicating the range of the imaging area on a first surface that is the front surface disposed on the side with the phosphor 5111 illustrated in FIGS. 57A-1, 57A-2, 57B-1, and 57B-2 and a second surface that is the rear surface disposed on the side with the pixel array 5112 illustrated in FIGS. 57A-1, 57A-2, 57B-1, and 57B-2. The medical practitioner can thus figure out by looking at the indicators 5113 and 5114 displayed on the front surface and the rear surface of the housing 5130 that the FPD imaging unit 5100 is capable of radiographic imaging at both the front and rear surfaces.

In FIGS. 59A and 59B, the imaging area exterior 5120 that is the thick section and the imaging area interior 5110 that is the thin section are illustrated to constitute the same plane to facilitate placement on a flat surface. However, the present exemplary embodiment is not limited thereto. The present exemplary embodiment is also applicable to a perspective view where grid mounting spaces 5160 are provided on both the front and rear of the FPD imaging unit 5100. The configuration illustrated in FIGS. 59A and 59B is desirable if usability and error prevention measures similar to those of conventional radiographic imaging apparatuses are desirably provided in an easy-to-understand manner. On the other hand, if the radiographic imaging apparatus is mainly used on rounds carts, such as beds, and less likely to be placed on a hard flat surface, and the front and rear surfaces of the FPD imaging unit 5100 are used for imaging with similar frequencies, the configuration where the grid mounting spaces 5160 are provided on the front and rear without the same plane is appropriate.

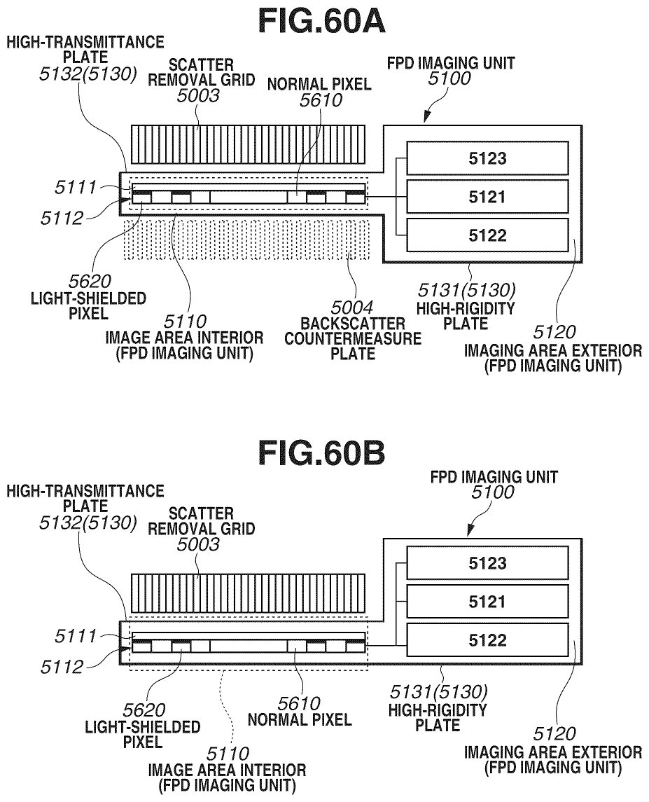

FIGS. 60A and 60B are diagrams illustrating cross-sectional examples of the FPD imaging unit 5100 illustrated in FIG. 55. Specifically, FIG. 60A illustrates a cross-sectional example where the grid mounting space 5160 illustrated in FIGS. 59A and 59B is provided on both the front and rear of the FPD imaging unit 5100. FIG. 60B illustrates a cross-sectional example where the grid mounting space 5160 is provided on only one side. In these FIG. 60A and FIG. 60B, components similar to those illustrated in FIG. 55 are denoted by the same reference numerals, and a detailed description thereof will be omitted.

In the cross-sectional example illustrated in FIG. 60A, the grid mounting spaces 5160 are present on both the front and rear of the FPD imaging unit 5100, whereby the scatter removal grid 5003 and a backscatter countermeasure plate 5004 are arranged, respectively. The mounting arrangement may thus be changed depending on whether the incident direction of the radiation 5002 is the front or rear of the FPD imaging unit 5100. Moreover, when the FPD imaging unit 5100 is mounted on a bed frame, an upright frame, or the like, backscattered radiation may cause artifacts in the image and overlapping of scattered radiation may result in blurring in the radiographic image if substances with large atomic numbers, such as metals, are unevenly present behind. The presence of the grid mounting spaces 5160 on both the front and rear of the FPD imaging unit 5100 enables the scatter removal grid 5003 to be disposed on the incident direction side of the radiation 5002. Moreover, an air gap, the backscatter countermeasure plate 5004, or the scatter removal grid 5003 to substitute for the backscatter countermeasure plate 5004 may be disposed on the side opposite to the incident direction of the radiation 5002.

Medical cassettes for the FPD imaging unit 5100 have a standard thickness specified by JIS (Z4905), ISO (4090), or the like. According to the standard dimensions of general radiographic cassettes, the thickness is specified to be 15 mm (+1 mm, −2 mm). Too thick a cassette is unable to be inserted into an upright frame or horizontal frame designed to standard dimensions. By contrast, the thickness of thinner cassettes is increased to a predetermined thickness by applying covers onto the cassettes. In the present exemplary embodiment, the imaging area interior 5110 and the imaging area exterior 5120 of the FPD imaging unit 5100 have different thicknesses, and the thickness of the imaging area interior 5110 is desirably 10 mm or less. The thickness of the scatter removal grid 5003 consists of the thickness of a lead foil portion and that of a covering material, often with a total of 3 mm or less. Here, the coating material has a thickness of approximately 0.5 mm. The thickness of the lead foil portion varies with the grid ratio, and is approximately 0.8 mm for 4:1, 1.2 mm for 6:1, and 2.0 mm for 10:1. Considering the maximum value of 16 mm in the standard dimensions of general radiographic cassettes and subtracting the total thickness of 6 mm when scatter removal grids 5003 with a maximum thickness of 3 mm are disposed on both sides, the thickness of the imaging area interior 5110 is desirably 10 mm or less. Configuring the imaging area interior 5110 with a thickness of 10 mm or less does not only achieve a reduction in thickness, but also produces a new effect that cannot be achieved solely by the combination of being able to be inserted into horizontal and upright frames designed to standard dimensions along with the grids.

Referring to FIGS. 60A and 60B, the need to use both high-rigidity material and high-transmittance material to meet the material requirements for the housing 5130 of the FPD imaging unit 5100 will be described. For example, if the phosphor 5111 such as CsI undergoes plastic deformation due to external force, the CsI columns are distorted to affect the image. The film or glass where the photoelectric conversion elements of the pixel array 5112 is arranged in an array may also be cracked or fissured under external force, which affects the radiographic image and the durability. In the present exemplary embodiment, the housing 5130 portion of the imaging area interior 5110 is desirably formed of high-rigidity material that does not easily transmit external force. On the other hand, the radiographic imaging apparatus 5000 is desirably capable of imaging with as low a dose of radiation 5002 as possible. In general, high-rigidity materials tend to have low radiation transmittance, and the surface portion of the housing 5130 where the radiation 5002 is incident is thus desirably formed of high-transmittance material. Although the price may be higher, CFRP (Carbon Fiber Reinforced Plastics) and the like can be said to be materials with both properties of high radiation transmittance and high rigidity. The front and rear surfaces of the housing 5130 of the FPD imaging unit 5100 are desirably formed of different materials, with the high-transmittance plate 5132 formed of high-radiation-transmittance material on the side with the phosphor 5111 and the high-rigidity plate 5131 formed of high-rigidity material on the side with the pixel array 5112.

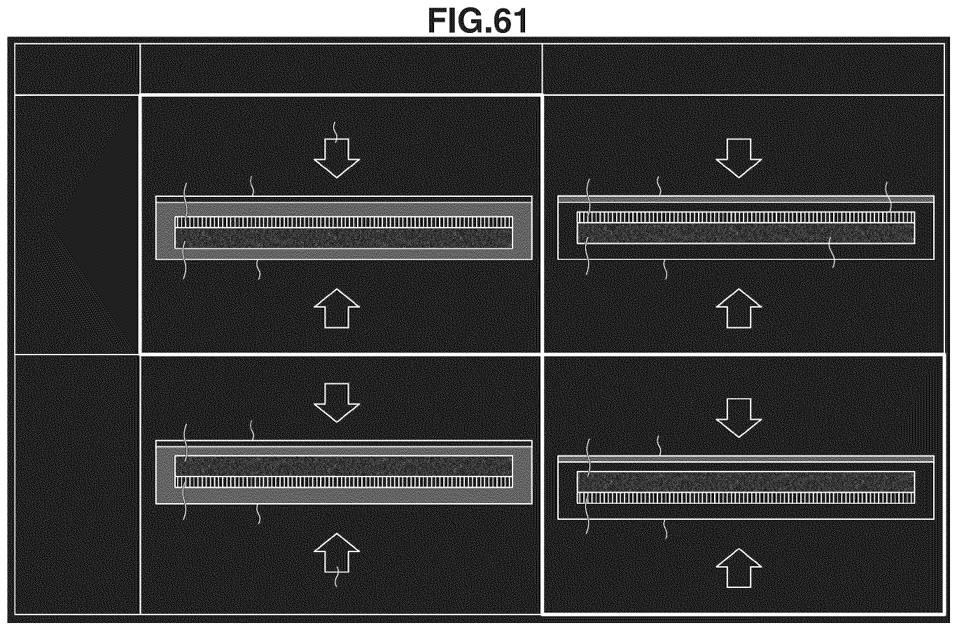

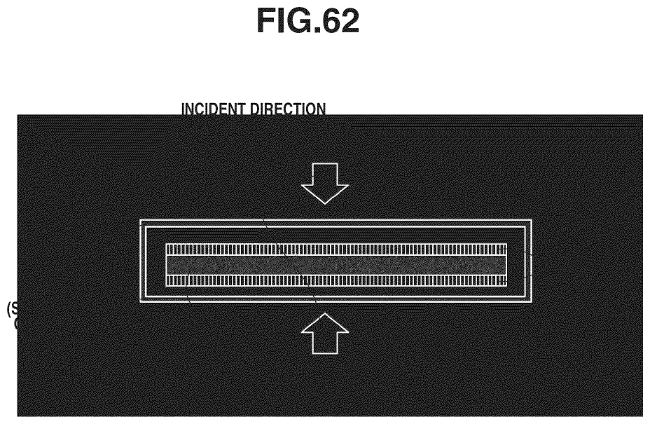

FIGS. 61 and 62 are diagrams illustrating configuration examples of the housing 5130 of the FPD imaging unit 5100 illustrated in FIG. 55. In these FIGS. 61 and 62, components similar to those illustrated in FIG. 55 are denoted by the same reference numerals, and a detailed description thereof will be omitted. FIG. 61 illustrates a matrix with the internal configuration of the FPD imaging unit 5100 on the vertical axis and the materials constituting the housing 5130 of the FPD imaging unit 5100 on the horizontal axis.

In FIGS. 61(a) and 61(c), the housing 5130 is constituted by high-transmittance material above and high-rigidity material below. In FIGS. 61(b) and 61(d), the housing 5130 is constituted by high-rigidity material above and high-transmittance material below. For example, the use of high-rigidity material for the side walls of the housing 5130 as illustrated in FIGS. 61(a) and 61(c) reduces thickness. By contrast, the use of high-transmittance material for the side walls of the housing 5130 has an advantage of weight reduction. However, since the radiation 5002 incident from the side walls of the housing 5130 should appropriately be reduced if possible, the side walls are more appropriately formed of high-rigidity material as illustrated in FIGS. 61(a) and 61(c). If high-transmittance material is disposed below as illustrated in FIGS. 61(b) and 61(d), an air gap (clearance) or a cushioning member against external force may be needed to prevent transmission of external force to the pixel array 5112 and the phosphor 5111. As compared to FIGS. 61(a) and 61(c), the thickness of the housing 5130 of the FPD imaging unit 5100 in FIGS. 61(b) and 61(d) is increased in a case where reflection in the entire rigidity is performed in accordance with the side wall structure.

Next, referring to the vertical axis of FIG. 61, FIGS. 61(a) and 61(b) illustrate configuration examples of the FPD imaging unit 5100 where the phosphor 5111 is disposed above and the pixel array 5112 is disposed below. FIGS. 61(c) and 61(d) illustrate configuration examples of the FPD imaging unit 5100 where the pixel array 5112 is disposed above and the phosphor 5111 is disposed below. It has been described with reference to FIGS. 57A-1, 57A-2, 57B-1, and 57B-2 that even when the same radiation 5002 is incident, the image quality characteristics of the radiographic image differ depending on which is disposed in the side where the radiation 5002 is incident, the phosphor 5111 or the pixel array 5112.

For example, in the case of front incidence where the phosphor 5111 is disposed in the side where the radiation 5002 is incident, the image quality characteristics of the radiographic image are high DQE and low MTF because of the mechanism described with reference to FIGS. 57A-1, 57A-2, 57B-1, and 57B-2.

The reason for the low MTF is that the occurrence of light emitting points 5311 on the phosphor incident side is probabilistically dominant. This results in a distance equivalent to the thickness of the phosphor (approximately 300 to 700 μm) before the visible light 5312 reaches the photoelectric conversion elements, and the light diffuses even when columnar phosphors are used.

On the other hand, in the case of rear incidence where the pixel array 5112 is disposed in the side where the radiation 5002 is incident, the image quality characteristics of the radiographic image are low DQE and high MTF because of the mechanism described with reference to FIGS. 57A-1, 57A-2, 57B-1, and 57B-2. The reason for the low DQE is that the radiation 5002 is transmitted through the pixel array 5112 before incident on the phosphor 5111, and the reached radiation 5002 decreases by approximately 1% to 3%. The reason for the high MTF is that the occurrence of light emitting points 5311 on the phosphor incident side is probabilistically dominant, and the amount of diffusion of the visible light 5312 is small since the distance between the light emitting points 5311 and the pixel array 5112 is small.

Next, suitable examples of the housing 5130 of the FPD imaging unit 5100 for medical use will be described with reference to FIG. 61. Even in medical applications, there are imaging that needs high sensitivity and imaging that needs high sharpness. For example, in pediatric and other imaging where a reduction in the exposure dose is demanded, high sensitivity is desired. In adult imaging where discerning finer structures is demanded, high precision is desired. In medical applications, the purpose of imaging, the imaging site, physique, age, previous imaging information, and the like are known in advance. It is therefore appropriate to display recommendations so that the front surface and the rear surface of the housing 5130 are appropriately selected. There are considered to be mainly two needs (1) and (2) as follows:
(1) The need to commercialize the product in a way that enhances the strength of the front and rear surfaces in terms of image quality; and
(2) The need to commercialize the product so that both the front and rear surfaces have the similar image quality.

In the case of the need (1), the configurations of the housing 5130 illustrated in FIGS. 61(a) and 61(d) are suitable, for example. These configurations are characterized in that high-transmittance material is disposed on the high DQE/low MTF side. By adopting the configurations of the housing 5130 in FIGS. 61(a) and 61(d), the apparatus performs imaging with high DQE, i.e., specialized for sensitivity during high DQE/low MTF imaging. Since high-rigidity material is used as well, the thin imaging area interior 5110 is relatively strong against external force.

In the case of the need (2), the phosphors 5111 are desirably disposed on both the front and rear sides as in the FPD imaging unit 5100 illustrated in FIG. 62, with the pixel array 5112 sandwiched between the phosphors 5111 on both sides. In such a case, the front surface and the rear surface of the housing 5130 are desirably formed of the same material, which is achieved by using a material that combines high transmittance for the radiation 5002 and high rigidity, such as CFRP. As illustrated in FIG. 62, an FPD imaging unit 5100 symmetrically configured provides radiographic images of the similar image quality regardless of which surface is irradiated with the radiation 5002, the front surface or the rear surface. This has the advantage that there is no need to change the imaging processing based on the incident direction of the radiation 5002, since the radiographic images on the front surface and the rear surface have the similar image quality. Examples of the high-rigidity material include iron, magnesium, cast aluminum alloys, ceramics, and metal-ceramic composite materials. Examples of the high-transmittance material include carbon. If the material satisfies both the high-rigidity plate 5131 and the high-transmittance plate 5132, the foregoing configuration is not necessarily restrictive. For example, reinforced CFRP has high rigidity due to the interweaving of carbon fibers despite carbon's low atomic number and high radiation transmittance. A material such as CFRP is suitable for use on both the front and rear surfaces. However, since the price is relatively high, it is appropriate to use a metal plate such as Mg, which has low radiation transmittance and high rigidity, for either the front or rear surface. In such a case, the configuration of the FPD imaging unit 5100 according to the present exemplary embodiment is desirably employed for implementation.

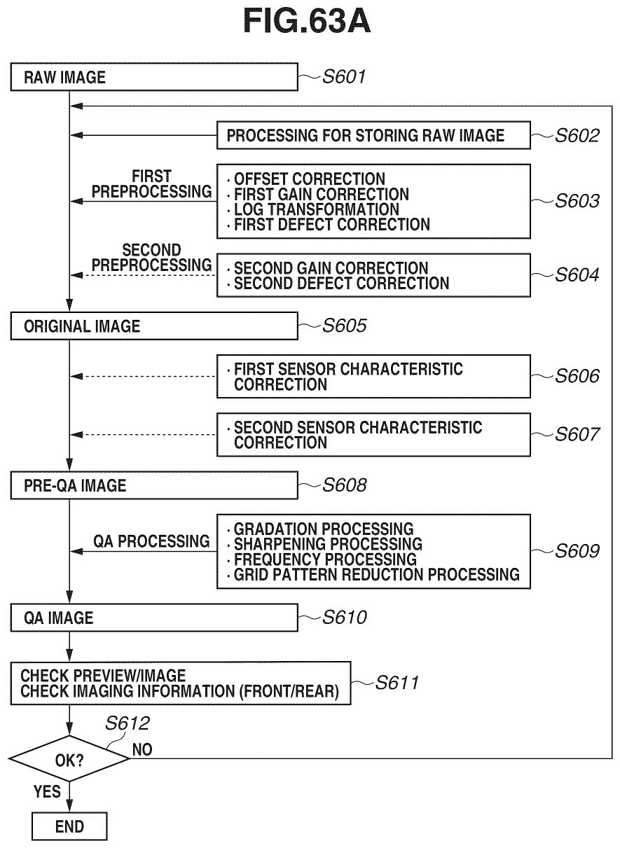

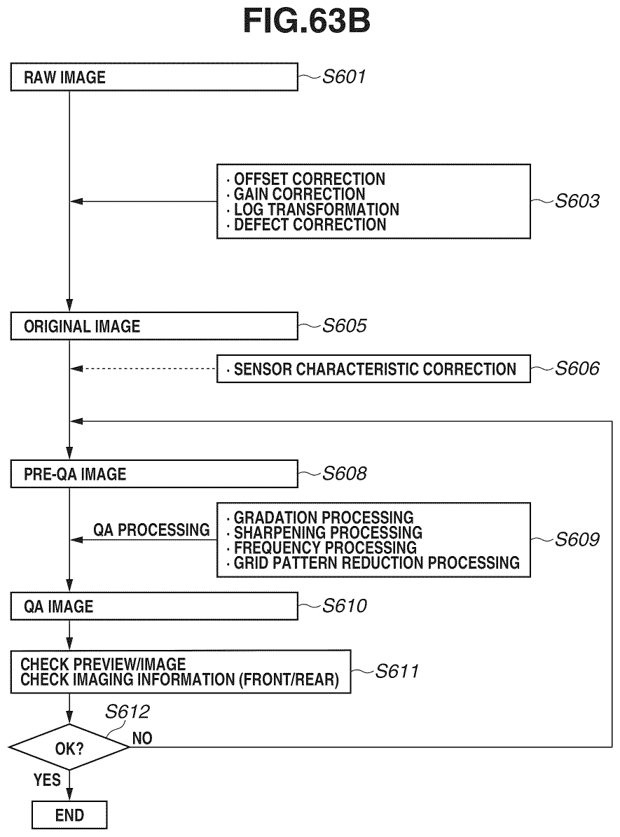

FIGS. 63A and 63B are flowcharts illustrates examples of processing procedures for a control method of the radiographic imaging apparatus 5000 according to the twenty-first exemplary embodiment and a comparative example. Specifically, FIG. 63A is a flowchart illustrating an example of the processing procedure for the control method of the radiographic imaging apparatus 5000 according to the twenty-first exemplary embodiment of the present invention. FIG. 63B is a flowchart illustrating an example of the processing procedure for the control method of the radiographic imaging apparatus according to the comparative example.

Processing common to those illustrated in FIGS. 63A and 63B, or equivalently, the processing according to the comparative example illustrated in FIG. 63B will be described.

Initially, in step S601 illustrated in FIG. 63B, the FPD imaging unit 5100 transmits a captured radiographic image to the CPU 5008 as a raw image.

Next, in step S603 illustrated in FIG. 63B, the preprocessing means 5010 performs preprocessing on the raw image. The preprocessing includes offset correction (dark image correction), gain correction (bright image correction), log transformation, and defect correction.

Next, in step S605 illustrated in FIG. 63B, the preprocessing means 5010 stores the preprocessed image as an original image.

Next, in step S606 illustrated in FIG. 63B, the radiographic imaging apparatus 5000 performs sensor characteristic correction processing specific to the type of FPD imaging unit 5100 on the original image. For example, if the MTF differs from one sensor to another, processing for making the sensors equivalent is performed. The reason is that if images with characteristics different from one sensor to another are subjected to QA processing, the appearance varies sensor by sensor and is difficult to adjust.

Next, in step S608 illustrated in FIG. 63B, the radiographic imaging apparatus 5000 sets the image subjected to the sensor characteristic correction processing as a pre-QA image. This pre-QA image is not an image easy for the medical practitioner such as a doctor to make a diagnosis. QA processing is therefore performed in the next step.

Next, in step S609 illustrated in FIG. 63B, the image processing means 5050 performs QA processing on the pre-QA image. Examples of this QA processing include gradation processing, sharpening processing, frequency processing, and grid pattern reduction processing. For example, in the case of a frontal chest image, the gradation processing involves applying an S-curve or the like to enhance the visibility of the lung fields and mediastinum and compress other density ranges. The sharpening processing is performed in examining peripheral blood vessels or observing trabecular patterns. The frequency processing emphasizes higher frequencies in observing bones, spicules, and the like, and emphasizes lower frequencies in observing tumors and the like for screening. The grid pattern reduction processing reduces a pattern caused by the grid frequency used and its aliasing frequencies.

Next, in step S610 illustrated in FIG. 63B, the image processing means 5050 sets the QA-processed image as a QA image.

Next, in step S611, the radiographic imaging apparatus 5000 displays a preview of the QA image on the image display means 5071, and has the medical practitioner perform a visual check. Here, the medical practitioner also checks the imaging information (for example, imaging direction [front or rear]).

Next, in step S612 illustrated in FIG. 63B, the radiographic imaging apparatus 5000 determines whether the check result of step S611 is OK. If, as a result of this determination, the check result of step S611 is not OK (unacceptable) (NO in step S612), the processing returns to step S608, and the radiographic imaging apparatus 5000 performs the processing of steps S608 onward.

On the other hand, if, as a result of the determination of step S612 illustrated in FIG. 63B, the check result of step S611 is OK (YES in step S612), the processing of the flowchart illustrated in FIG. 63B ends.

Next, the processing according to the twenty-first exemplary embodiment of the present invention illustrated in FIG. 63A will be described.

After the acquisition of the raw image in step S601 illustrated in FIG. 63A, then in step S602 illustrated in FIG. 63A, processing for storing the raw image is performed.

Next, in step S603 illustrated in FIG. 63A, the preprocessing means 5010 performs first preprocessing on the raw image. The first preprocessing includes offset correction (dark image correction), first gain correction (bright image correction), log transformation, and first defect correction.

Next, in step S604 illustrated in FIG. 63A, the preprocessing means 5010 performs second preprocessing on the image subjected to the first preprocessing. The second preprocessing includes second gain correction (bright image correction) and second defect correction.

Next, in step S605 illustrated in FIG. 63A, the preprocessing means 5010 stores the image subjected to the second preprocessing as an original image.

Next, in step S606 illustrated in FIG. 63A, like step S606 of FIG. 63B, the radiographic imaging apparatus 5000 performs sensor characteristic correction processing (first sensor characteristic correction processing) specific to the type of FPD imaging unit 5100 on the original image.

Next, in step S607 illustrated in FIG. 63A, the radiographic imaging apparatus 5000 performs second sensor characteristic correction processing on the original image. Details of the second sensor characteristic correction processing illustrated in this FIG. 63A will be described below.

Next, in step S608, the radiographic imaging apparatus 5000 sets the image subjected to the second sensor characteristic correction processing as a pre-QA image.

Next, in step S609 illustrated in FIG. 63A, the image processing means 5050 performs the QA processing on the pre-QA image.

Next, in step S610 illustrated in FIG. 63A, the image processing means 5050 sets the QA-processed image as a QA image.

Next, in step S611 illustrated in FIG. 63A, the radiographic imaging apparatus 5000 displays a preview of the QA image on the image display means 5071, and has the medical practitioner perform a visual check. Here, the medical practitioner also checks the imaging information (for example, imaging direction [front or rear]).

Next, in step S612 illustrated in FIG. 63A, the radiographic imaging apparatus 5000 determines whether the check result of step S611 is OK. If, as a result of this determination, the check result of step S611 is not OK (unacceptable) (NO in step S612), the processing returns to step S602, and the radiographic imaging apparatus 5000 performs the processing of steps S602 onward.

On the other hand, if, as a result of the determination of step S612 illustrated in FIG. 63A, the check result of step S611 is OK (YES in step S612), the processing of the flowchart illustrated in FIG. 63A ends.

In the processing according to the twenty-first exemplary embodiment of the present invention illustrated in FIG. 63A, the imaging information (for example, imaging direction [front or rear]) is checked during the visual check of the QA image in step S611. If the QA processing by the image processing means 5050 is reverse between the front and rear surfaces of the FPD imaging unit 5100, there still is room to generate a more appropriate radiographic image. Then, in the processing according to the twenty-first exemplary embodiment of the present invention illustrated in FIG. 63A, if, as a result of the determination of step S612, the check result of step S611 is not OK (unacceptable) (NO in step S612), the processing needs to return to step S602. The reason is that the gain correction may be performed using different gain maps between the front surface and the rear surface of the FPD imaging unit 5100, or defect map correction may not be performed with appropriate coordinates if the phosphor 5111 has flaws and the FPD imaging unit 5100 is set with the wrong side up. While it would certainly be acceptable to reverse-transform the processes of the image, such processing takes long and does not necessarily restore the original image in a reversible manner.

In the processing according to the twenty-first exemplary embodiment of the present invention illustrated in FIG. 63A, the raw image is stored in step S602. It is therefore also appropriate to return to the raw image of step S602 if the FPD imaging unit 5100 is set with wrong side up. Then, in steps S603 and S604, the first preprocessing and the second preprocessing are performed. In the case where the incident direction of the radiation 5002 is different between the front and rear surfaces of the FPD imaging unit 5100, the second preprocessing for the front and rear surfaces of the FPD imaging unit 5100 is performed based on the incident direction of the input actual radiation 5002. This second preprocessing includes gain correction processing and defect correction processing, for example.

In step S607, the second sensor characteristic correction based on the actual physical properties of the sensors on the front or rear surface is performed on the original image obtained in step S605 since the incident direction of the radiation differs between the front and rear surfaces of the FPD imaging unit 5100.

In step S609, the QA processing is performed on the pre-QA image obtained in step S608. Then, in step S611, the radiographic image is visually checked again.

In the examples illustrated in FIGS. 63A and 63B, step S611 includes the image check processing. In fact, the dose index value (EI value) is often calculated as well, using the pixel values of the image. Even when the reached dose is the same, the pixel values of the raw image may be different depending on whether the incident direction of the radiation 5002 is the front or rear of the FPD imaging unit 5100. The pixel values for the dose are desirably corrected based on the physical properties of the front or rear sensors of the actual FPD imaging unit 5100. In this regard, the flowchart according to the present exemplary embodiment may be applied to not only images but also analysis functions using pixel values, such as the dose index value (EI value). The flowchart according to the present exemplary embodiment described with reference to FIG. 63A is configured to absorb differences between the physical properties of the front and rear sensors of the FPD imaging unit 5100 at the stage before the pre-QA image. The flowchart may be configured so that the dose index value (EI value) and the like are separately corrected. If only the image is concerned, the values for adjusting the strength, frequency, and the like of the QA processing may be switched between the front and rear surfaces of the FPD imaging unit 5100 to perform adjustments at a stage after the pre-QA image.

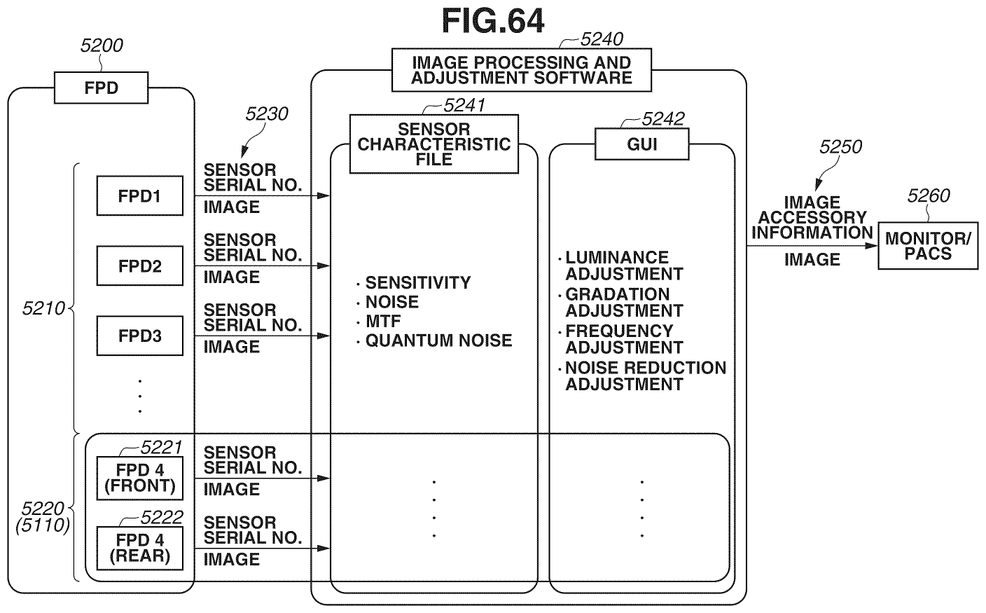

FIG. 64 is a diagram illustrating image processing examples of the image processing means 5050 according to the twenty-first exemplary embodiment and a comparative example. FIG. 64 illustrates a procedure for processing radiographic images captured by FPDs 5200 and serial numbers 5230 using image processing and adjustment software 5240 in the CPU 5008, and outputting the processed radiographic images and the like 5250 to a monitor/PACS 5260. Note that the image processing and adjustment software 5240 is implemented outside the FPDs 5200, but may be implemented inside the FPDs 5200.

There is a plurality of pieces of information input to the image processing and adjustment software 5240. In FIG. 64, the FPDs 5200 are divided into FPDs 5210 capable of imaging on only one side of the FPDs 5200 as a comparative example, and an FPD 5220 capable of imaging on both the front and rear surfaces of the FPD 5220 as the twenty-first exemplary embodiment. From the perspective of the image processing and adjustment software 5240, the FPD 5220 capable of imaging on both the front and rear surfaces of the FPD 5200 may be regarded as two sensors 5221 and 5222. In other words, the two sensors 5221 and 5222 have the same serial number but may be handled as models with separate sensor physical properties since the physical properties of the sensors differ between the front and rear surfaces.

The image processing and adjustment software 5240 stores sensor characteristic files 5241 for respective models or individuals. Specifically, the sensor characteristic files 5241 store, for example, the sensitivity, noise, MTF, quantum noise, and the like of each model or individual. The image processing means 5050 selects a sensor characteristic file 5241 suitable for the FPD 5200 having captured an image based on the transmitted serial number 5230 of the sensor and/or input or detected front/rear information, and performs image processing.

The image processing and adjustment software 5240 has a GUI 5242 from which the user can perform brightness adjustment, gradation adjustment, frequency adjustment, noise reduction adjustment, etc. The user makes the adjustments while viewing the image, and if an appropriate image is obtained, outputs the image to the monitor/PACS 5260. In FIG. 64, from the perspective of the image processing and adjustment software 5240, the front sensor 5221 and the rear sensor 5222 are processed as respective separate FPDs 5200. However, the image processing and adjustment software 5240 may be configured to perform the image processing calculations by assigning different serial numbers 5203.

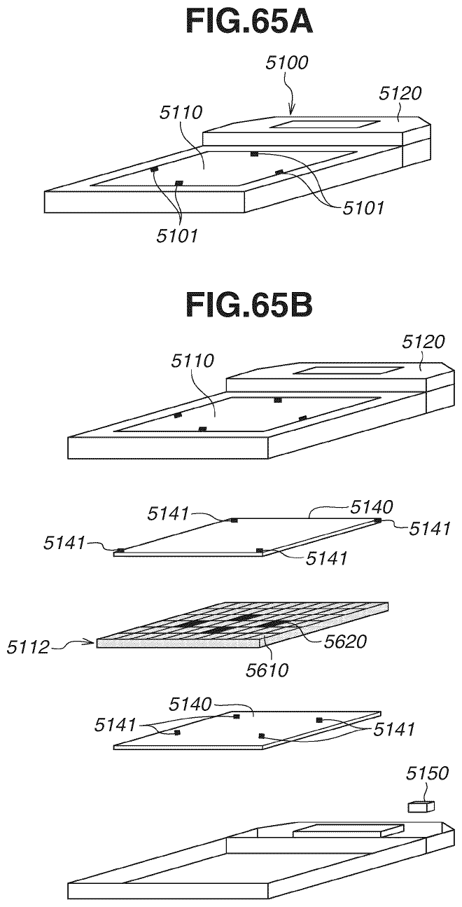

FIGS. 65A and 65B are diagrams illustrating an example of the appearance and internal configuration of the FPD imaging unit 5100 illustrated in FIG. 55. In these FIGS. 65A and 65B, components similar to those illustrated in FIG. 55 are denoted by the same reference numerals, and a detailed description thereof will be omitted. Specifically, FIGS. 65A and 65B illustrate a configuration example for automatically inputting the detected incident direction of the radiation 5002 with respect to the FPD imaging unit 5100. In FIGS. 65A and 65B, the detected incident direction of the radiation 5002 is automatically input, but the medical practitioner may manually input the detected incident direction of the radiation 5002 with respect to the FPD imaging unit 5100.

FIG. 65A is a diagram illustrating an example of the appearance of the housing of the FPD imaging unit 5100. The structure for detecting the incident direction of the radiation 5002 is desirably built in the housing of the FPD imaging unit 5100, but may be disposed outside the housing of the FPD imaging unit 5100. As an example where the structure for detecting the incident direction of the radiation 5002 is disposed outside the housing of the FPD imaging unit 5100, in FIG. 65A, front markers 5101 are disposed on the imaging area interior 5110 outside the housing. In such a case, the detected incident direction of the radiation 5002 is automatically input by analyzing the radiographic image based on the radiation 5002 with which the imaging area interior 5110 including the front markers 5101 is irradiated.

FIG. 65B is a diagram illustrating an example of the internal configuration of the FPD imaging unit 5100 illustrated in FIG. 55. Specifically, FIG. 65B illustrates a part of the internal configuration of the imaging area interior 5110 of the FPD imaging unit 5100 in an exploded manner. If cushioning materials 5140 are disposed on the inside of the housing, for example, the front and rear sides of the pixel array 5112, the front markers 5141 may be attached to the cushioning members 5140. However, such a method has a disadvantage that the positions of the front markers are visible in the radiographic image. In view of this, it is desirable that an acceleration measurement element 5150 using a piezoelectric element be included in the housing, the position of the radiation generation means 5001 be calibrated in advance, and whether the incident direction of the radiation 5002 is from the front or the rear be determined using the acceleration measurement element 5150. Alternatively, whether the incident direction of the radiation 5002 is from the front or the rear may be determined using the light-shielded pixels 5620 that are masked by a light-shielding mask at either one or both the front and rear sides of the pixel array 5112. As illustrated in FIG. 65B, the inclusion of not only the normal pixels 5610 but also the light-shielded pixels 5620 in the pixel array 5112 enables the determination of the incident direction of the radiation 5002. To accurately determine whether the incident direction of the radiation 5002 is the front or rear, the light-shielded pixels 5620 or front markers are desirably arranged at least one in 500×500 pixels across the entire pixel array 5112, so that the incident direction is detected even when the irradiation field is narrowed. Moreover, since it is rare for the radiation 5002 to not hit the center area of the pixel array 5112 during imaging, the arrangement is desirably sparse in the peripheral area of the pixel array 5112 and dense in the center area of the pixel array 5112. While the three radiation incident direction determination methods using the front markers, the acceleration measurement element 5150, and the light-shielded pixels 5620 have been described with reference to FIGS. 65A and 65B, one of the radiation incident direction determination methods may be used alone. The medical practitioner may input using the manual input means 5061.

FIGS. 66A, 66A-1, 66A-2, 66B, 66B-1, and 66B-2 are diagrams and charts illustrating the twenty-first exemplary embodiment and intended to describe the radiation incident direction determination method using the light-shielded pixels 5620 illustrated in FIGS. 65A and 65B. In these FIGS. 66A, 66A-1, 66A-2, 66B, 66B-1, and 66B-2, components similar to those illustrated in FIGS. 55, 65A, and 65B are denoted by the same reference numerals, and a detailed description thereof will be omitted.

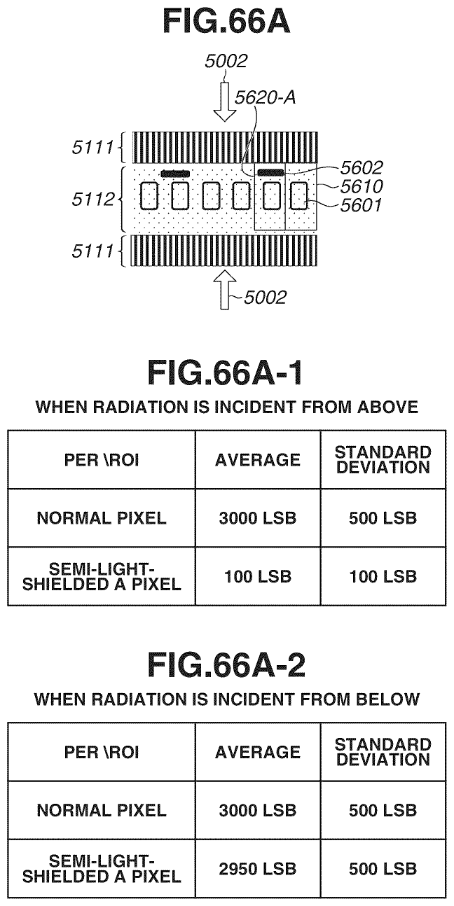

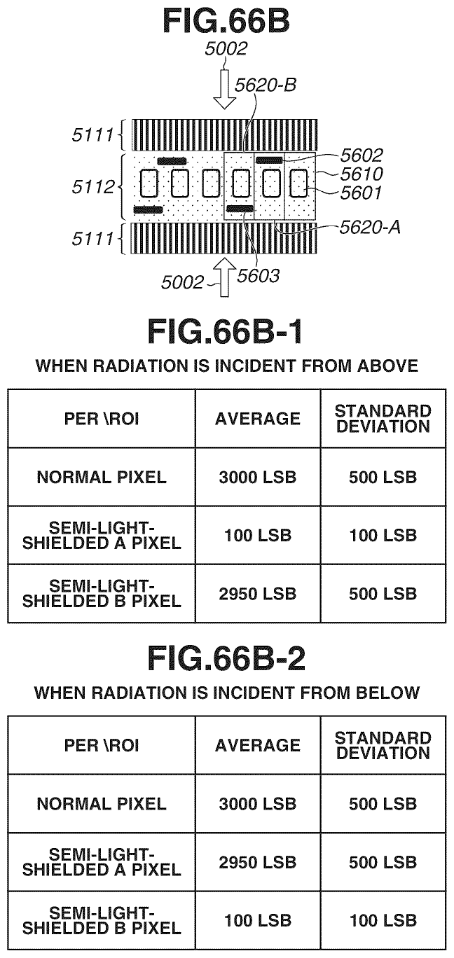

In FIG. 66A, the pixel array 5112 includes normal pixels 5610 each including a photoelectric conversion element 5601, and light-shielded pixels 5620-A each including a photoelectric conversion element 5601 and a light-shielding mask 5062 disposed above the photoelectric conversion element 5601. The light-shielded pixels 5620-A are light-shielded pixels 5620 where the photoelectric conversion elements 5601 are shielded from light incident from above.

In FIG. 66B, the pixel array 5112 includes normal pixels 5610, light-shielded pixels 5620-A, and light-shielded pixels 5620-B each including a photoelectric conversion element 5601 and a light-shielding mask 5603 disposed below the photoelectric conversion element 5601. The light-shielded pixels 5620-B are light-shielded pixels 5620 where the photoelectric conversion elements 5601 are shielded from light incident from below.

FIGS. 66A and 66B illustrate examples where the phosphors 5111 are formed both above and below the pixel array 5112. However, the phosphor 5111 may be formed on only one side. FIGS. 66A and 66B illustrate the radiation 5002 as being incident from both above and below, whereas only one side, either the upper side or the lower side, is irradiated at a time.

The pixels arranged in an array form in the pixel array 5112 include the photoelectric conversion elements 5601. The light-shielding mask 5602 has a structure such that light easily enters from either above or below, rather than a structure that does not allow light to enter the interior of the light-shielded pixels 5620-A at all. Since the photoelectric conversion layers of the photoelectric conversion elements 5601 have sensitivity to obliquely incident light as well, the light-shielding masks 5602 and 5603 are desirably larger than the photoelectric conversion elements 5601 in area, and desirably formed in an L-shape. However, in the present exemplary embodiment, the light shielding by the light-shielding masks 5602 and 5603 does not need to be complete. Since it is only necessary to be able to statistically determine the incident direction of the radiation 5002, a light-shielding rate of, e.g., 50% or so is sufficient to determine the incident direction of the radiation 5002.

The example of FIG. 66A will be described.

For example, in a case where the radiation 5002 is incident from above, the statistics of the outputs of the light-shielded pixels 5620-A that are semi-shieled by the light-shielding mask 5602 and the statistics of the outputs of the normal pixels 5610 as illustrated in FIG. 66A-1 are obtained. In a case where the radiation 5002 is incident from below, the statistics of the outputs of the light-shielded pixels 5620-A and the statistics of the outputs of the normal pixels 5610 as illustrated in FIG. 66A-2 are obtained. As illustrated in FIGS. 66A-1 and 66A-2, the statistics (average and standard deviation) of the normal pixels 5610 and the statistics (average and standard deviation) of the light-shielded pixels 5620-A differ significantly depending on the incident direction of the radiation 5002. While the statistics (average and standard) are described here, only the statistics (average) may be sufficient. The greater the number of light-shielded pixels 5620-A, the higher the statistical stability is and the more irradiation field collimators is accommodated. The statistical stability also increases in cases where the subject H has a complex structure, or the pixel values of adjoining pixels of the subject H vary slightly as with the scatter removal grid 5003. However, the fewer the more desirable, since the light-shielded pixels 5620-A become defective pixels in the image.

In FIG. 66B, the light-shielded pixels 5620-A provide upper light shielding, and the light-shielded pixels 5620-B provide lower light shielding. Specifically, FIG. 66B is a chart for describing a method for determining the incident direction of the radiation 5002 with upper and lower, two-sided light shielding. The principle is similar to that of FIG. 66A described above. In FIG. 66B, the light-shielding masks 5602 and 5603 are disposed both above and below the photoelectric conversion elements 5601, which has a disadvantage of increased semiconductor manufacturing processes. However, if the radiographic imaging apparatus 5000 are configured to be capable of imaging on both the front and rear surfaces for other functional purposes, and the light-shielding masks 5602 and 5603 are disposed both above and below the photoelectric conversion elements 5601, the following processing is performed. That is, the light-shielded pixels 5620-A and the light-shielded pixels 5620-B illustrated in FIG. 66B are subjected to respective statistical processes for the light-shielded pixels 5620-A and the light-shielded pixels 5620-B separately. This may improve robustness even when the radiographic image varies because of precision, the subject H, or the irradiation field. While FIGS. 66A-1, 66A-2, 66B-1, and 66B-2 illustrate semi-light-shielded pixels, light-shielded pixels implemented for other purposes may be used. Examples of the other purposes include using fully light-shielded pixels used for dark current correction in images and the AEC function built in the FPD imaging unit 5100, etc. Such fully light-shielded pixels are also included in the light-shielded pixels 5620 according to the present exemplary embodiment.

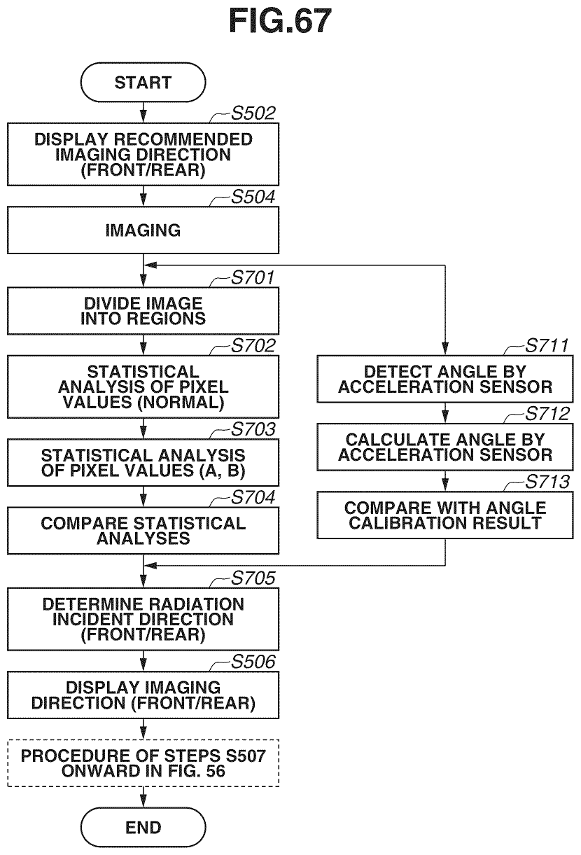

FIG. 67 is a flowchart illustrating a processing procedure for the radiation incident direction detection processing by the radiographic imaging apparatus 5000 illustrated in FIG. 55. In this FIG. 67, processing steps similar to those illustrated in FIG. 56 are denoted by the same step numbers, and a detailed description thereof will be omitted.

In the processing of the flowchart illustrated in FIG. 67, the imaging conditions are determined in advance. In step S502, the radiographic imaging apparatus 5000 therefore initially displays whether the recommended imaging direction is the front (phosphor side) or the rear (pixel array side) on the operation panel 5060 or the imagine display means 5071. The medical practitioner then places the radiographic imaging apparatus 5000 based on the display of the recommended imaging direction (front or rear).

Next, in step S504, the radiographic imaging apparatus 5000 causes the radiation generation means 5001 to generate the radiation 5002, and causes the FPD imaging unit 5100 to capture a radiographic image of the subject H.

In performing statistical processing on the light-shielded pixels 5620 and the normal pixels 5610, the radiographic image captured in step S504 has variations in the pixel values of even the normal pixels 5610 depending on the location within the image due to factors such as the distribution of generation by the radiation generation means 5001 and the structure of the subject H. In step S701, the radiographic imaging apparatus 5000 therefore divides the radiographic image captured in step S504 into regions and performs calculations on the assumption that pixel values are equivalent within the same image region or at nearby locations.

Next, in step S702, the radiographic imaging apparatus 5000 performs statistical analysis of the pixel values of the normal pixels 5610.

Next, in step S703, the radiographic imaging apparatus 5000 performs statistical analysis of the pixel values of the light-shielded pixels 5620-A and 5620-B.

The statistical calculations of the foregoing pixel values are performed within the same regions of the radiographic image, using averages and standard deviations.

Next, in step S704, the radiographic imaging apparatus 5000 compares the statistical analyses of both the normal pixels 5610 and the light-shielded pixels 5620. Since there is an obvious statistical difference between the front and rear surfaces, statistical significance testing is not needed. In the next step S705, the radiographic imaging apparatus 5000 determines the radiation incident direction (front or rear).

Next, in step S506, the radiographic imaging apparatus 5000 displays the imaging direction (front or rear) that is the incident direction of the radiation 5002 on the image display means 5071 or the operation panel 5060. The processing of steps S507 and onward in FIG. 56 is then performed.

The present exemplary embodiment is not limited to the radiation incident direction determination using the light-shielded pixels 5620. For example, the radiation incident direction determination may be performed based on the acceleration measurement element 5150 using the piezoelectric element. Receiving acceleration, the acceleration measurement element 5150 generates an electric charge depending on the direction. In step S711 of FIG. 67, the acceleration measurement element 5150 obtains a relative angle as appropriate by performing measurement each time and calculating the integral value of the generated charges.

Next, in step S712, the radiographic imaging apparatus 5000 calculates a relative angle from an initial value based on the obtained integral value.

Next, in step S713, the radiographic imaging apparatus 5000 makes a comparison with the result of angle calibration to the radiation generation means 5001 after power-on before imaging. The relative angle between the radiation generation means 5001 and the radiographic imaging apparatus 5000 at the time of the radiographic imaging is thereby figured out. In the present exemplary embodiment, it is sufficient to figure out whether the imaging is performed on the front or rear surface, and an accuracy in units of 1° is not required. The drawback of the acceleration measurement element 5150 is that the angle is only a relative one and difficult to calculate when the radiographic imaging apparatus 5000 is moved with its power off. The angle is also difficult to calculate by the radiographic imaging apparatus 5000 alone when the radiation generation means 5001 is moved. Measuring an angle relative to geomagnetism, like a gyro sensor, is also appropriate. However, in hospitals where there may be MRI devices nearby, calibration before angle measurement is a prerequisite for ensuring accuracy.

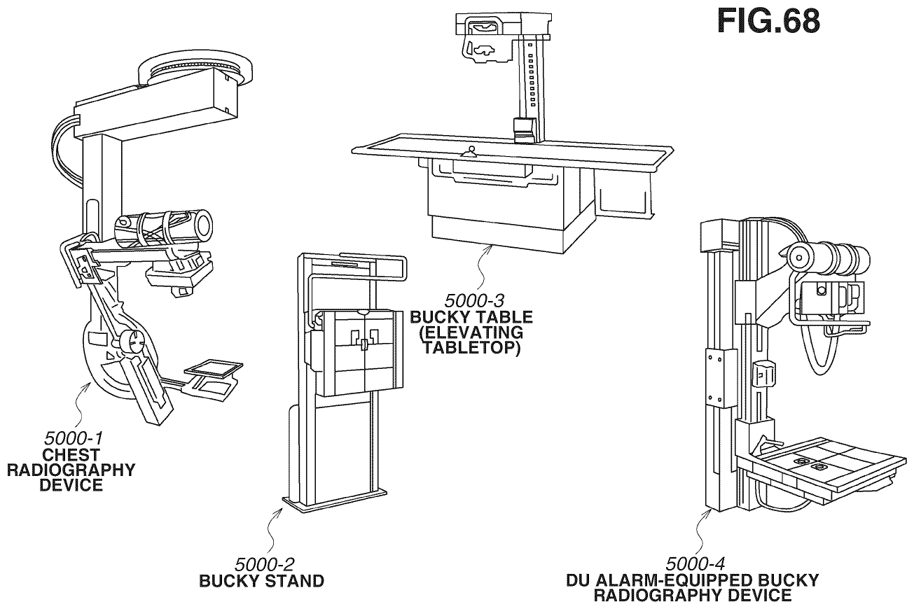

FIG. 68 is a diagram illustrating specific examples of imaging systems to which the radiographic imaging apparatus 5000 according to the twenty-first exemplary embodiment is applicable. The radiographic imaging apparatus 5000 according to the present exemplary embodiment may be mounted on a chest radiography device 5000-1, a Bucky stand 500-2, an elevatable Bucky table 5000-3, and a DU alarm-equipped Bucky radiography device 5000-4 illustrated in FIG. 68, for example.

The radiographic imaging apparatus 5000 according to the present exemplary embodiment includes, in its imaging area interior 5110 within the range of the imaging area to be irradiated with the radiation 5002, the phosphor 5111 that converts the radiation 5002 into light and the pixel array 5112 where the plurality of pixels including the photoelectric conversion elements 5601 is arranged. The radiographic imaging apparatus 5000 also includes, in its imaging area exterior 5120 outside the range of the imaging area to be irradiated with the radiation 5002, the printed board including electronic parts that communicate with the pixel array 5112. The radiographic imaging apparatus 5000 according to the present exemplary embodiment further includes the housing 5130 that accommodates the phosphor 5111, the pixel array 5112, and the printed board. The indicators 5113 and 5114 indicating the range of the imaging area to be irradiated with the radiation 5002 during imaging are displayed on the first surface of the housing 5130 disposed on the side with the phosphor 5111 and the second surface disposed on the side with the pixel array 5112.

According to such a configuration, the printed board is disposed in the imaging area exterior 5120. Even if the first and second surfaces of the housing 5130 are incorrectly situated, the printed board is thus prevented from being visible in the captured radiographic image. Moreover, since the indicators 5113 and 5114 indicating the range of imaging area to be irradiated with the radiation 5002 during imaging are displayed on the first and second surfaces of the housing 5130, the medical practitioner can figure out that radiographic imaging is able to be performed on both the first and second surfaces of the housing 5130. This can reduce the frequency of reimaging of the subject H when the incident direction of the radiation 5002 with respect to the imaging area of the radiographic imaging apparatus is changed.

Furthermore, the image processing means 5050 according to the present exemplary embodiment performs different image processing between the radiographic image obtained based on the radiation incident on the imaging area from the first surface of the housing 5130 and the radiographic image obtained based on the radiation incident from the second surface of the housing 5130.

According to such a configuration, even if the incident direction of the radiation 5002 with respect to the imaging area of the radiographic imaging apparatus is changed, deterioration in the image quality of the radiographic image is suppressed, and the frequency of reimaging of the subject H is reduced.

### Other Exemplary Embodiments

The present invention may also be implemented by processing for supplying a program for implementing one or more functions of the foregoing exemplary embodiments to a system or an apparatus via a network or a storage medium, and reading and executing the program by one or more processors in a computer of the system or apparatus. A circuit that implements one or more functions (such as an ASIC) may also be used for implementation.

This program and a computer-readable storage medium storing the program are included in the present invention.

The foregoing twenty-first exemplary embodiment of the present invention is merely an example of specific implementation for carrying out the present invention, and the technical scope of the present invention should not be interpreted as being limited to this. In other words, the present invention may be practiced in various forms without departing from the technical concept or essential features thereof.

The twenty-first exemplary embodiment of the present invention includes the following configurations.

### [Configuration 70]

A radiographic imaging apparatus configured to detect incident radiation and capture a radiographic image, the radiographic imaging apparatus comprising:
a phosphor configured to convert the radiation into light, the phosphor being disposed within a range of an imaging area to be irradiated with the radiation;
a pixel array configured to have a plurality of pixels including a photoelectric conversion element for converting the light into an electrical signal of the radiographic image, the pixel array being disposed within the range of the imaging area;
a printed board configured to include an electronic part for communicating with the pixel array, the printed board being disposed outside the range of the imaging area; and
a housing configured to accommodate the phosphor, the pixel array, and the printed board,
wherein an index indicating the range of the imaging area is displayed on a first surface and a second surface of the housing, the first surface being disposed at a position closer to the phosphor, the second surface being disposed at a position closer to the pixel array.

### [Configuration 71]

The radiographic imaging apparatus according to Configuration 70, further including image processing means configured to perform different image processing between the radiographic image obtained based on the radiation that has been incident on the imaging area from the first surface, and the radiographic image obtained based on the radiation that has been incident on the imaging area from the second surface.

### [Configuration 72]

The radiographic imaging apparatus according to Configuration 71, further including detection means configured to detect whether an incident direction of the radiation with respect to the imaging area is a first incident direction from the first surface or a second incident direction from the second surface,
wherein the image processing means performs the image processing based on a detection result of the detection means.

### [Configuration 73]

The radiographic imaging apparatus according to Configuration 72, wherein the first incident direction and the second incident direction are opposite directions.

### [Configuration 74]

The radiographic imaging apparatus according to Configuration 72 or 73, wherein the detection means is configured to detect whether the incident direction is the first incident direction or the second incident direction, based on an incident angle of the radiation input from automatic input means or manual input means.

### [Configuration 75]

The radiographic imaging apparatus according to Configuration 74, wherein the automatic input means includes at least one or more of:
first input means using a light-shielded pixel among the plurality of pixels arranged in the pixel array, the light-shielded pixel including a light-shielding mask for shielding the photoelectric conversion element from the incident light;
second input means using an acceleration measurement element including a piezoelectric element; and
third input means using a marker disposed within the range of the imaging area.

### [Configuration 76]

The radiographic imaging apparatus according to any one of Configurations 72 to 75, further including direction display means configured to display a recommended incident direction between the first incident direction and the second incident direction, based on an imaging order obtained before the imaging.

### [Configuration 77]

The radiographic imaging apparatus according to Configuration 76, further including warning display means configured to, if the recommended incident direction and the incident direction of the radiation during the imaging are different, display a warning.

### [Configuration 78]

The radiographic imaging apparatus according to any one of Configurations 72 to 77, further including storage means configured to store an image quality characteristic value of the radiographic images obtained based on the radiation incident in the first incident direction and the second incident direction,
wherein the image processing means selects the image quality characteristic value for the first incident direction or the image quality characteristic value for the second incident direction, based on the detection result of the detection means, and performs the image processing based on the selected image quality characteristic value.

### [Configuration 79]

The radiographic imaging apparatus according to Configuration 78, wherein the image quality characteristic value is at least one of a pixel value dependent on a dose of the radiation, a noise value dependent on the dose of the radiation, and a sharpness value dependent on a frequency of the radiographic image.

### [Configuration 80]

The radiographic imaging apparatus according to any one of Configurations 72 to 79, wherein the image processing means performs the different image processing by changing a parameter of the imaging processing.

### [Configuration 81]

The radiographic imaging apparatus according to Configuration 80, wherein the image processing means includes, as means configured to change the parameter of the imaging processing, at least one of:
first changing means for changing a noise reduction processing parameter for the radiographic image;
second changing means for changing a frequency processing parameter for the radiographic image;
third changing means for changing a gradation processing parameter for the radiographic image; and
fourth changing means for changing a grid pattern reduction processing parameter for the radiographic image.

### [Configuration 82]

The radiographic imaging apparatus according to any one of Configurations 70 to 81, wherein the housing is formed of a high radiation transmittance material in a portion close to the phosphor, and formed of a high rigidity material in a portion close to the pixel array.

### [Configuration 83]

The radiographic imaging apparatus according to any one of Configurations 70 to 82,
wherein the housing has different thicknesses within the range of the imaging area and outside the range of the imaging area, and
wherein the thickness within the range of the imaging area is 10 mm or less.

According to the foregoing Configurations 70 to 83, the frequency of reimaging of the subject when the incident direction of the radiation on the radiographic imaging apparatus is changed is reduced.

The present invention is not limited to the above-described exemplary embodiments, and various modifications and variations may be made without departing from the spirit and scope of the present invention. Accordingly, the following claims are appended to disclose the scope of the invention.

This application claims the benefit of Japanese Patent Applications No. 2022-165498, filed Oct. 14, 2022, No. 2022-172565, filed Oct. 27, 2022, No. 2023-063673, filed Apr. 10, 2023, No. 2023-071786, filed Apr. 25, 2023, No. 2023-119938, filed Jul. 24, 2023, and No. 2023-171786, filed Oct. 3, 2023, which are hereby incorporated by reference herein in their entirety.

According to the present invention, a radiographic imaging apparatus that has an appropriate shape and enables appropriate operation in consideration of the user's workability is provided.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A radiographic imaging apparatus comprising:
a radiation detection panel configured to include an effective imaging area where incident radiation is detected;
a control substrate configured to control driving of the radiation detection panel;
a processing substrate configured to process a signal output from the radiation detection panel; and
a housing configured to accommodate the radiation detection panel, the control substrate, and the processing substrate,
wherein the housing includes
a first thickness section, which has a first thickness in an incident direction of the radiation, and where the effective imaging area is disposed, and
a second thickness section, which has a second thickness greater than the first thickness in the incident direction of the radiation, and where the control substrate and the processing substrate are disposed, and
wherein the control substrate and the processing substrate are disposed to overlap each other at least in part when the second thickness section is viewed along the incident direction of the radiation.

2. A radiographic imaging apparatus comprising:
a radiation detection panel configured to include an effective imaging area where incident radiation is detected;
a control substrate configured to control driving of the radiation detection panel;
a housing configured to accommodate the radiation detection panel and the control substrate; and
a grip portion configured to be gripped to hold the housing,
wherein the housing includes
a first thickness section, which has a first thickness in an incident direction of the radiation, and where the effective imaging area is disposed, and
a second thickness section, which has a second thickness greater than the first thickness in the incident direction of the radiation, and where the control substrate and the grip portion are disposed, and
wherein the control substrate and the grip portion are disposed to overlap each other at least in part when the second thickness section is viewed along the incident direction of the radiation, and the control substrate is disposed at a position closer to a side where the radiation is incident than the grip portion.

3. A radiographic imaging apparatus comprising:
a radiation detection panel configured to include an effective imaging area where incident radiation is detected;
a control substrate configured to control driving of the radiation detection panel;
a flexible circuit board configured to connect the radiation detection panel and the control substrate; and
a housing configured to accommodate the radiation detection panel, the control substrate, and the flexible circuit board,
wherein the housing includes a first thickness section, which has a first thickness in an incident direction of the radiation, and where the effective imaging area is disposed, a second thickness section, which has a second thickness greater than the first thickness in the incident direction of the radiation, and where the control substrate is disposed, and a gradient section, which connects the first thickness section and the second thickness section with a gradient, and where at least a part of the flexible circuit board is disposed, and
wherein the flexible circuit board connects the radiation detection panel and the control substrate which are disposed at different positions in the incident direction of the radiation, with a gradient.

4. The radiographic imaging apparatus according to claim 1, further comprising a battery configured to be disposed in the second thickness section of the housing and to supply power to the radiographic imaging apparatus,
wherein the control substrate and the battery are disposed to overlap at least in part when the second thickness section is viewed along the incident direction of the radiation.

5. The radiographic imaging apparatus according to claim 1, wherein the radiation detection panel and the control substrate are disposed at different positions in the incident direction of the radiation.

6. The radiographic imaging apparatus according to claim 1, wherein the second thickness section is thicker than the first thickness section toward a side where the radiation is incident.

7. The radiographic imaging apparatus according to claim 1, wherein the processing substrate is one or more.

8. The radiographic imaging apparatus according to claim 1, wherein the control substrate is disposed at a position close to a side where the radiation is incident, with respect to the control substrate.

9. The radiographic imaging apparatus according to claim 8, wherein in a direction perpendicular to the incident direction of the radiation, the processing substrate is extended toward a position where the radiation detection panel is disposed, compared to the control substrate.

10. The radiographic imaging apparatus according to claim 1, further comprising a shielding member configured to reduce electromagnetic noise, the shielding member being disposed between the control substrate and the processing substrate.

11. The radiographic imaging apparatus according to claim 1, further comprising a grip portion configured to be disposed in the second thickness section of the housing and to be gripped to hold the housing,
wherein the grip portion and the processing substrate are disposed without overlapping when the second thickness section is viewed along the incident direction of the radiation.

12. The radiographic imaging apparatus according to claim 1, further comprising a battery configured to be disposed in the second thickness section of the housing and to supply power to the radiographic imaging apparatus,
wherein the battery and the processing substrate are disposed without overlapping when the second thickness section is viewed along the incident direction of the radiation.

13. The radiographic imaging apparatus according to claim 1, further comprising:
a grip portion configured to be disposed in the second thickness section of the housing and to be gripped to hold the housing; and
a battery configured to be disposed in the second thickness section of the housing and to supply power to the radiographic imaging apparatus,
wherein the processing substrate and the battery are disposed with the grip portion therebetween when the second thickness section is viewed along the incident direction of the radiation.

14. The radiographic imaging apparatus according to claim 1, further comprising wiring configured to connect the control substrate and the processing substrate,
wherein in the control substrate and the processing substrate, the wiring is disposed on a side opposite to a side close to a position where the radiation detection panel is disposed.

15. The radiographic imaging apparatus according to claim 2, wherein the grip portion is formed in a recessed shape in a surface of a side of the second thickness section where the radiation is incident.

16. The radiographic imaging apparatus according to claim 2, wherein the grip portion is formed in a recessed shape in a surface of a side of the second thickness section opposite to a side where radiation is incident.

17. A radiographic imaging system comprising:
the radiographic imaging apparatus according to claim 1; and
a radiation generation apparatus configured to generate the radiation.

18. A radiographic imaging apparatus comprising:
a radiation detection panel configured to include an effective imaging area where radiation transmitted through a subject is detected;
a predetermined circuit configured to detect a signal output from the radiation detection panel; and
a housing configured to accommodate the radiation detection panel and the predetermined circuit.
wherein the housing includes a first thickness section, which has a first thickness in an incident direction of the radiation, and where at least the effective imaging area is disposed, and a second thickness section, which has a second thickness greater than the first thickness in the incident direction of the radiation, and where at least the predetermined circuit is disposed, and
wherein in the second thickness section, a current reduction mechanism for reducing a loop current in a region where a closed circuit may occur is disposed.

19. A radiographic imaging apparatus comprising:
a radiation detection panel configured to include an effective imaging area where incident radiation is detected;
a housing configured to accommodate the radiation detection panel; and
a display unit configured to function as a user interface,
wherein the housing includes a first thickness section, which has a first thickness in an incident direction of the radiation, and where the effective imaging area is disposed, and a second thickness section, which has a second thickness greater than the first thickness in the incident direction of the radiation, and where the display unit is disposed in an area which is excluded from a center in a longitudinal direction and is on one end side in the longitudinal direction.

20. A radiographic imaging apparatus comprising:
a radiation detection panel configured to include an effective imaging area where radiation transmitted through a subject is detected;
a sensor unit configured to include one or more types of sensors for detecting the subject; and
a housing configured to accommodate the radiation detection panel,
wherein the housing includes a first thickness section, which has a first thickness in an incident direction of the radiation, and where the effective imaging area is disposed, and a second thickness section, which has a second thickness greater than the first thickness in the incident direction of the radiation, and where the sensor unit is disposed.

21. A radiographic imaging apparatus comprising:
a radiation detection panel configured to include an effective imaging area where radiation transmitted through a subject is detected; and
a housing configured to accommodate the radiation detection panel,
wherein in the housing, an index indicating a range of the effective imaging area is disposed on a first surface corresponding to a surface on one side of the radiation detection panel and a second surface corresponding to a surface on the other side of the radiation detection panel.
